(19) United States
(12) Patent Application Publication
(10) Pub. No.: US 2023/0100105 A1
(43) Pub. Date: Mar. 30, 2023

(54) REGULATORY ELEMENTS IN THE GENOME

(71) Applicant: The Jackson Laboratory, Bar Harbor, ME (US)

(72) Inventors: Chia-Lin Wei, Farmington, CT (US); Chew Yee Ngan, Farmington, CT (US)

(73) Assignee: The Jackson Laboratory, Bar Harbor, ME (US)

(21) Appl. No.: 17/801,570

(22) PCT Filed: Feb. 23, 2021
(86) PCT No.: PCT/US2021/019182

§ 371 Date: Aug. 23, 2022

Related U.S. Application Data
(60) Provisional application No. 62/980,342, filed on Feb. 23, 2020.

(51) Int. Cl. C12Q 1/6886; C12Q 1/6804

(52) U.S. Cl. CPC C12Q 1/6886; C12Q 1/6804; C12Q 2600/158; C12Q 2600/136; C12Q 1/6869

(57) ABSTRACT

The invention, in part, relates to assessing interactions between gene transcription enhancers and gene transcription repressor, identifying agents that modulate transcription, and use of methods and identified agents to prevent and treat diseases and conditions, such as cancers.

### RELATED APPLICATIONS

This application claims the benefit under 35 U.S.C. § 119(e) of U.S. Provisional application Ser. No. 62/980,342 filed Feb. 23, 2020, the disclosure of which is incorporated by reference herein in its entirety.

### FIELD OF THE INVENTION

The invention, in part, relates to assessing interactions between gene transcription enhancers and gene transcription repressor, identifying agents that modulate transcription, and use of methods and identified agents to prevent and treat diseases and conditions, such as cancers.

### BACKGROUND OF THE INVENTION

In metazoan development, lineage specific gene expression is modulated by the delicate balance between transcription activation and repression. Despite much knowledge in the art relating to enhancer-centered transcription activation, silencers and their roles in normal development remain poorly understood.

### SUMMARY OF THE INVENTION

According to an aspect of the invention, a method of identifying a candidate agent for modulating a condition is provided, the method including: (a) detecting, with a means comprising a ChIA-PET method, a gene regulation system (GRS), wherein an activity of the GRS comprises a physical interaction between a transcription-factor binding (TFB) element, a transcription factor (TF) complex, and a gene modifier element in a cell; (b) identifying a target gene whose transcription is modified by the activity of the GRS as a condition-inducer gene or a condition-suppressor gene; (c) determining an effect of the activity of the GRS on repression and de-repression of the transcription of the identified target gene, wherein: (i) if the target gene is a condition-suppressor gene and the effect of the activity of the GRS is de-repression of the transcription of the target gene, the activity of the GRS inhibits the condition; (ii) if the target gene is a condition-suppressor gene and the effect of the activity of the GRS is repression of the transcription of the target gene, reducing the activity of the GRS inhibits the condition; (iii) if the target gene is a condition-inducer gene and the effect of the activity of the GRS is repression of the transcription of the target gene, the activity of the GRS inhibits the condition; (iv) if the target gene is a condition-inducer gene and the effect of the activity of the GRS is de-repression of the transcription of the target gene, reducing the activity of the GRS inhibits the condition; and (d) identifying one or more candidate agents that modify the activity of the GRS. In some embodiments, the TFC includes 1, 2, 3, or more subunits, wherein the physical interaction between the TFB element, the TF complex, and the gene modifier element includes an interaction between at least one of the subunits and the TFB element and an interaction between at least one of the subunits and the gene modifier element. In certain embodiments, the TFB element is a gene silencer element. In some embodiments, the TF complex includes a polycomb repressor complex 2 (PRC2). In some embodiments, the GRS activity transcriptionally represses expression of the target gene. In certain embodiments, the TFB element is a gene activator element. In some embodiments, the GRS activity transcriptionally de-represses expression of the target gene. In certain embodiments, the condition includes at least one of: a cancer, cell differentiation, cell de-differentiation, embryonic development, development, organ development, cell death, cell division, and a genetic disease. In some embodiments, the condition is a cancer and the target gene is a cancer-inducer gene. In some embodiments, the condition is a cancer and the target gene is a cancer suppressor gene. In certain embodiments, the method also includes identifying a function of the identified target gene. In some embodiments, the condition is ovarian cancer. In some embodiments, the condition is a metastatic cancer. In some embodiments, the cell is a cancer cell. In certain embodiments, the condition is cell differentiation. In some embodiments, the cell is obtained from a subject. In certain embodiments, the cell is obtained from a cell culture. In some embodiments, the method also includes determining a level of transcription of the target gene and optionally comparing the determined level to a control level of transcription. In some embodiments, the method also includes determining an effect of one of the candidate agent on the determined level of transcription of the target gene, and optionally comparing the determined level to a control level of transcription. In certain embodiments, determining the effect of one of the candidate agents includes contacting the cell with a composition that includes the candidate agent. In some embodiments, the method also includes testing one or more of any of the aforementioned identified candidate agents as a determination of the presence or absence of an inhibitory effect of the agent(s) on the condition. In some embodiments, the method also includes testing one or more of the aforementioned identified candidate agents as a determination of the presence or absence of an enhancing effect of the agent(s) on the condition.

According to another aspect of the invention, a method of identifying a candidate agent for inhibiting a cancer is provided, the method including: (a) detecting, with a means comprising a ChIA-PET method, a gene regulation system (GRS), wherein an activity of the GRS comprises a physical interaction between a transcription-factor binding (TFB) element, a transcription factor (TF) complex, and a gene modifier element in a cancer cell; (b) identifying a target gene whose transcription is modified by the activity of the GRS as a cancer-inducer gene or a cancer-suppressor gene; (c) determining an effect of the activity of the GRS on repression and de-repression of the transcription of the identified target gene, wherein: (i) if the target gene is a cancer-suppressor gene and the effect of the activity of the GRS is de-repression of the transcription of the target gene, the activity of the GRS inhibits the cancer; (ii) if the target gene is a cancer-suppressor gene and the effect of the activity of the GRS is repression of the transcription of the target gene, reducing the activity of the GRS inhibits the cancer; (iii) if the target gene is a cancer-inducer gene and the effect of the activity of the GRS is repression of the transcription of the target gene, the activity of the GRS inhibits the cancer; (iv) if the target gene is a cancer-inducer gene and the effect of the activity of the GRS is de-repression of the transcription of the target gene, reducing the activity of the GRS inhibits the cancer; and (d) identifying one or more candidate agents that modify the activity of the GRS. the aforementioned identified candidate agents the TFC includes 1, 2, 3, or more subunits, wherein the physical interaction between the TFB element, the TF complex, and the gene modifier element includes an interaction between at least one of the subunits and the TFB element and an interaction between at least one of the subunits and the gene modifier element. In certain embodiments, the TFB element is a gene silencer element. In some embodiments, the TF complex includes a polycomb repressor complex 2 (PRC2). In some embodiments, the GRS activity transcriptionally represses expression of the target gene. In some embodiments, the TFB element is a gene activator element. In certain embodiments, the GRS activity transcriptionally de-represses expression of the target gene. In some embodiments, the target gene is a cancer-inducer gene. In certain embodiments, the target gene is a cancer suppressor gene. In some embodiments, the method also includes identifying a function of the identified target gene. In some embodiments, the cancer cell is an ovarian cancer cell. In some embodiments, the cancer cell is a metastatic cancer cell. In certain embodiments, the cancer cell is obtained from a subject. In some embodiments, the cancer cell is obtained from a cell culture. In some embodiments, the method also includes determining a level of transcription of the target gene and optionally comparing the determined level to a control level of transcription. In certain embodiments, the method also includes determining an effect of one of the candidate agent on the determined level of transcription of the target gene, and optionally comparing the determined level to a control level of transcription. In some embodiments, determining the effect of one of the candidate agents includes contacting the cancer cell with a composition comprising the candidate agent. In certain embodiments, the method also includes testing the one or more of any of the aforementioned candidate agents as a determination of the presence or absence of an inhibitory effect of the agent(s) on the cancer.

### BRIEF DESCRIPTION OF THE DRAWINGS

FIG. 1A-D presents schematic diagrams and graphs illustrating use of ChIA-PET analysis to define the PRC2 interactome in mESCs. FIG. 1A is a schematic diagram illustrating cross-linked chromatin fragmented and subjected to proximity ligation followed by ChIP enrichment for three core PRC2 components, EED, EZH2 and SUZ12 in mESC. Five billion read pairs were pooled to define PRC2 binding sites and interactions supported by PRC2 binding at both anchors (BA) and single anchor (SA). FIG. 1B shows profiles of interactions (upper tracks) and binding (lower tracks) across chr17:85,366,518-86,405,710 region for EED, EZH2, SUZ12, and combined PRC2 displayed with matching gene track. FIG. 1C shows BA and SA interactions across chr16:96,921,289-98,008,954 region together with the PRC2 binding profile and the associated genes. Y-axis shows the interaction frequency represented by the number of PET counts. FIG. 1D (upper panel) shows distribution of PRC2 BA interactions among nuclear compartments A, B and across A-B. Percent of total BA interactions are shown. FIG. 1D (lower panel) illustrates ChIA-PET interactions within 6 Mb of chromosomes 17 and 19, shown in reference with the topological associated domains (TADs) defined by Hi-C contact maps.

FIG. 2A-C plots and interaction profiles and a histogram illustrating reproducibility of PRC2 ChIA-PET analysis. FIG. 2A illustrates Pearson correlation coefficient, r, between individual ChIA-PET replicates for EED, EZH2, SUZ12 and the combined PRC2 libraries between three subunits. FIG. 2B shows PRC2 chromatin interactions and binding profile across chr4:139,536,779-140,286,920. Tracks from the top: BA interaction, PRC2 binding profiles and SA interactions. Y-axis: interaction frequency represented by PET counts. FIG. 2C is a histogram showing distribution of interaction frequency among BA and SA interactions. FIG. 3A-E presents schematic diagrams and graphs illustrating PRC2 mediating extensive chromatin looping in genes of low transcription activities. FIG. 3A illustrates four major subclasses of PRC2 interactions classified based on features, gene (G), promoter (P) and intergenic (I), associated with the interaction anchors. The chromosomal regions shown are as follows; P-P, chr8:91,651,961-92,862,573; P-G, chr2:155,604,301-155,765,282; P-I, chr5:66,963,794-67,352,967 and Intra-G looping, chr10:42,916,485-43,260,546. PRC2 binding profiles are shown in lower tracks. FIG. 3B illustrates distribution of interaction frequency (PET counts) across the gene coding regions associated with PRC2 intra-G looping (n=3,483). FIG. 3C provides a histogram showing percentages of genes with PRC2 interactions detected. X-axis indicates the protein factors bound at the promoters. Significant differences (paired t-test, p=0.0012) are found between binding in the presence (black) or absence (hatched) of RNAPII. FIG. 3D shows distribution of steady-state RNA expression level (FPKM) among genes with different patterns of binding and interactions. FIG. 3E is a schematic diagram and chart showing percentages of PRC2 tethered genes with single, dual, three or all four subclasses of interaction types. Most genes are associated with more than one category of interactions.

FIG. 4A-C provides interaction profiles, a histogram, and a schematic diagram illustrating extensive chromatin interactions between distal regulator elements (DREs) and PRC2 bound genes. FIG. 4A provides examples of the multiple co-occurred chromatin looping patterns (P-P, P-G, P-I and intra-G interactions) in the Wnt6-Ihh (chr1:74,751,523-74,968,999) and Hoxb (chr11:96,161,617-96,425,610) regions are shown from EED (red), EZH2 (purple), SUZ12 (blue) and PRC2 (black) ChIA-PET libraries, respectively. FIG. 4B shows percentages of genes exhibit single, 2-type, 3-type and all 4-type of interactions. For example, among the 4,372 genes with P-P interactions, 14% of them have all 4-type of interactions (P-P, P-I, P-G and intra-G looping). FIG. 4C is a schematic diagram of a proposed model on how DREs can connect to their target genes and function as either enhancers or silencers by binding to RNAPII or PRC2.

FIG. 5A-F presents interaction profiles and contact heat maps demonstrating that intergenic anchors function as transcriptional silencers. In FIG. 5A chromatin interaction profiles within chr9:37,071,610-37,689,270 mediated by each subunits of PRC2 are shown together with connected genes, H3K27me3 and CTCF binding intensity. The 10 Kb deleted si-Δchr9 region is highlighted. FIG. 5B shows contact heat maps of chromosome 9 in wild type (WT) and si-Δchr9 KO mESC lines. Regions (3-60 Mb) surrounding the deleted locus are highlighted. FIG. 5C provides PRC2-mediated chromatin interaction profiles within chr9:36,955,506-37,955,721 in two independent WT and si-Δchr9 KO mESC lines. Lower panel displays region surrounding si-Δchr9 locus (chr9:37,395,678-37,576,659). FIG. 5D illustrates expression changes between connected vs. non-connected genes within 500 kb and 1 Mb of the si-Δchr9 region. FIG. 5E illustrates RNA expression of selected genes connected to the si-Δchr9 locus from WT (n=3), F1 (n=2) and G9 (n=3) KO mESC clones. FIG. 5F illustrates differential gene expression changes between the wild type and homozygous deleted clones F1 shown as a volcano plot. Selected genes with the most striking upregulation are labelled.

FIG. 6A-B provides schematic diagrams illustrating experimental validation of intergenic silencers in vivo. FIG. 6A is a schematic overview of generating heterozygous founder mice strains and ES clones carrying deletion in the intergenic anchors by CRISPR/Cas9. FIG. 6B is a schematic description of genotype strategy and primer design used in screening of KO mice and derived ES clones.

FIG. 7A-F presents graphs and photomicrographic images demonstrating that mice with PRC2-bound silencer deletion display pleiotropic developmental defects. FIG. 7A is a bar graph showing relative ratio of −/−, −/+ and +/+ genotypes determined in six KO F2 crosses, including attempts from multiple crosses. FIG. 7B is a histogram including si-Δchr9, numbers of embryos at E9.5 days (Y-axis) of different genotypes (X-axis) from F2 crosses with heterozygous KO locus. FIG. 7C illustrates morphology of wild type (+/+) and homozygous (−/−) si-Δchr9 embryos at E9.5. FIG. 7D is a bar graph of numbers of phenotypic assays with significant changes among the eight domains detected in each of the five deletion with viable homozygous KOs. Abbreviations; bodycmp: body composition; cbc: complete blood count; ekg: electrocardiography; gtt: glucose tolerance test; grip: grip strength; ldbox: light-dark box test; oft: open field test; ppi: prepulse inhibition test. Graphs of FIG. 7E show significant alteration in bone density and plasma glucose detected in si-Δchr5 and si-Δchr11 KO mice, respectively. FIG. 7F shows percent of hits in the PRC2-silencer KO (n=5) mice relative to these detected in the KO of protein coding genes (n=730).

FIG. 8A-E presents graphs, heat maps, and a schematic illustration of showing that intergenic anchors exhibit the poised chromatin state and acquire enhancer signature during differentiation. FIG. 8A shows fold enrichment of four histone modifications, RNAPII and CTCF binding over input control across ±10 Kb of intergenic (I)-anchor regions. In horizontal center of graph, top trace is H3K27me3, second from top trace is H3K4me1, third from top trace is RNAPII, fourth from top trace is H3K27ac, fifth from top trace is CTCF, and lowest trace is H3K9me3. FIG. 8B shows heat maps of H3K27ac, H3K27me3 and H3K9me3 normalized enrichment of the 1,800 I-anchors throughout progressive developmental stages in forebrain. The color scales represented the fold enrichment of ChIP over input. FIG. 8C illustrates enhancer activities of the PRC2 bound intergenic anchors in Nkx2-5 and Dlx3/4 loci observed in developing mouse embryos (heart in upper panel, mm1645 and hindbrain in lower panel, mm568) (www.enhancer.lbl.gov). FIG. 8D shows four distinct patterns of I-anchors based on the clustering of H3K27ac signal profiles across 74 different developmental stages collected from 12 tissues. The color scales represented the fold enrichment of ChIP over input. FIG. 8E is a schematic diagram of a model of how PRC2 associated repressive chromatin foci contribute to TGS and transition into tissue specific enhancers during differentiation. PRC2 aggregated clusters are formed by extensive chromatin looping between silenced genes and their corresponding DREs. Upon differentiation, they are selectively dissolved, presumably in the absence of PRC2 binding. DREs acquire tissue specific enhancer signal and associate with RNAPII to active their target gene expression.

FIG. 9 provides interaction profiles demonstrating intergenic anchors deleted in the mouse KO strains by CRISPR-Cas9. PRC2 interactions and binding profiles from 5 of the 6 KO regions (si-Δchr9 is shown in FIG. 3A). Selective genes connected by the KO regions through the PRC2 loops are labelled. Chromosome location (from top to bottom) are as follow; chr11:118,861,894-119,194,521, chr5:28,100,320-28,484,061, chr3:107,423,514-107,782,737, chr7:143,061,554-143,537,289 and chr2:18,568,747-19,024,016.

FIG. 10A-D presents heat maps, interaction profiles and histograms demonstrating that the loss of connectivity triggers genes reactivation. FIG. 10A is a heat map showing connectivity in previous study using Hi-C and current study using ChIA-PET. Example shown is chr1:36,282,810-192,258,731. FIG. 10B shows results of topological-associated domain analysis, which showed no difference in si-Δchr9, si-Δchr7 compared to wild type. FIG. 10C shows loss of connecting loops in si-Δchr7 clones D4 and F4. Shown are chr7:142,557,623-14,3646,256 and zoom in region chr7:143,127,114-14,3550,277. FIG. 10D illustrates results showing genes expression of connected of si-Δchr7 and non-connected genes from flanking 500 kb and 1 Mb regions.

FIG. 11A-B provides interaction profiles and box graphs showing upregulation of genes associated with si-Δchr7. FIG. 11A illustrates PRC2 interaction and binding profiles of the 1 Mb Igf2/Kcnq1 imprinting region. The si-Δchr7 (chr7:143,440,438-143,450,716) is marked in red. Three of the 10 genes with P-I interactions to this KO region located 15.5 Mb upstream. FIG. 11B provides normalized RNA-seq counts of the connected genes in wild type (+/+) and 2 independent homozygous KO (−/−) ES clones D4 and F4. Gm44732 has no expression.

FIG. 12A-C provides a Venn diagram, plots and a circus plot illustrating upregulation of genes associated with si-Δchr9. FIG. 12A left-hand side shows a Venn diagram of differentially upregulated genes in si-Δchr9 clones F1 and G9. Differentially expressed genes in homozygous KO (−/−) ES clones G9 compared with wild type (+/+) ESC shown in volcano plot FIG. 12A, right-hand side (p-value vs. fold change). Dysregulated genes found in both F1 and G9 F1 only and G9 only are labelled. Selected genes with the most striking upregulation are labelled. FIG. 12B is a circos plot showing the inter-chromosomal connectivity (iPET counts >10) between the KO allele with the 29 upregulated gene loci. FIG. 12C provides graphs showing the distribution of interaction frequencies between the si-Δchr9 KO silencer locus and random background #1 (Left) or #2 (Right). TIFs between si-Δchr9 and the dysregulated genes are shown as lines.

FIG. 13A-B provides graphs of histone profiles of PRC2 interaction anchors. FIG. 13A shows fold enrichment fold of four histone modifications, RNAPII and CTCF binding over input across ±10 Kb of promoter (P) and Gene (G)-anchor regions. FIG. 13B shows enrichment of H3K4me3 and ATAC-seq profile across ±10 Kb of the promoter (P), gene (G) and intergenic (I) interaction anchors.

FIG. 14A-B provides heat maps and box plots. FIG. 14A shows heat maps H3K27me3, H3K27ac, H3K9me3 normalized signals of the 1,800 I-anchors through progressive developmental stages of kidney, limbs, hindbrain and liver. The color scales represented the fold enrichment of the ChIP vs input at log 2 scale. FIG. 14B shows graphs indicating expression of eRNA in distal regulatory elements (DREs) and those overlapped with PRC2-bound silencers.

### BRIEF DESCRIPTION OF THE SEQUENCES

SEQ ID NO: 1 is agatcggaagagc.

SEQ ID NO: 2-122 are shown in Table 1.

SEQ ID NO: 123-134 are shown Table 6.

### DETAILED DESCRIPTION

Polycomb repressive complex 2 (PRC2) is a key regulator inducing transcriptional gene silencing and chromatin interaction analyses of PRC2 have now been performed to identify silencers and their associated chromatin connectivity. Deletion of silencers in mice results in transcriptional de-repression of differentiation genes and embryonic lethality. While functioning as silencers in pluripotent cells, silencers can transition into active enhancers during development, suggesting their regulatory versatility. Integrative analysis of three-dimensional genome organization and spatial clusters of PRC2-chromatin hubs has now revealed the compact assembly as the structural basis of the silencing compartments. As described herein, the molecular nature of silencers and their associated chromatin architectures have now been determined, and certain methods described herein can be used to identify means and agents for altering transcription for treatment of diseases and conditions such as, but not limited to cancers. Certain embodiments of methods of the invention comprise identifying and using candidate agents to treat a disease or condition, such as a cancer, cell differentiation, etc.

Key elements important in mechanisms of gene regulation in eukaryotes comprise transcription factor binding (TFB) elements, transcription factor (TF) complexes, and gene modifier elements, the interaction of which modulates transcription of a target gene. The elements, complexes, and their interactions are components of what is referred to herein as a “gene regulation system” or GRS. The interactions of GSRs are involved in temporal regulation of gene transcription in cells and are important factors in regulating cell identity. TFB elements such as enhancer elements and silencer elements are involved in temporal and cell type-specific activation and deactivation of gene expression and have a role in regulating cell growth and cell differentiation.

### Gene Regulatory Systems

In some aspects of the invention methods are provided to identify agents capable of selectively regulating gene transcription in cells and subjects. Agents that modulate regulation of gene transcription can alter transcription of genes and be used to treat a disease or condition associated with a level of transcription of the gene. For example, the onset of a cancer, a developmental disorder, and/or other disease or condition may result from a level of transcription of a particular gene, and an agent capable of modulating regulation of the particular gene's transcription can be used to inhibit the onset of the cancer, developmental disorder, and/or other disease or condition, respectively. An agent capable of modulating regulation of a particular gene's transcription may be an agent that increases a level of the gene's transcription or may be an agent that decreases a level of the gene's transcription. An agent may be used to modify activity of a gene regulation system (GRS). The term GRS, as used herein means a system through which transcription of a particular gene is regulated, wherein by modulating an activity of the GRS one can modulate transcription of the particular gene.

A GRS comprises the following components that physically interact: (1) a transcription factor binding (TFB) element, (2) a transcription factor complex (TFC), and (3) a gene modifier element. As used herein the term “transcription factor binding element” means a genetic element, the activity of which regulates transcription of a specific gene, which may also be referred to herein as a “target” gene.

A TFB element may be referred to in the art as a: “gene activator” or “gene silencer” element. A TFB element may also be referred to in the art as a non-coding distal regulatory element (DRE) that directs transcription repression or de-repression of a gene. Transcriptional enhancer elements and silencer elements are cis-regulatory sequences that recruit transcription factor (TF) elements and are centrally involved in regulating transcription of their target gene(s), including regulating characteristics such as, but not limited to: timing of the start of transcription, timing of the end of transcription, and the level of transcription of their target gene(s). See, for example, Maston, G. A., et al., (2006) Ann. Rev. Genomics Hum. Genet. 7, 29-59, the content of which is incorporated by reference herein. A TFB element may, but need not be, in close physical proximity its target gene. In some embodiments, a TFB element is physically distant from its target gene. Enhancers and silencers communicate with their target gene(s) through physical interactions with their gene modifier elements, mediated by transcription factor complexes.

As used herein the term: “transcription factor complex” refers to a complex of molecules positioned between a TBF and a gene modifier element. A TF complex interacts with other GRS elements to regulate transcription of a target gene so the GRS target gene is expressed in the right cell at the right time. A TF complex may function alone, or two or more TF complexes may function in conjunction with other to direct activities such as, but not limited to: cell division, cell growth, cell-migration, and cell death. A TF complex comprises at least one DNA-binding domain (DBD), which attaches to a specific sequence of DNA that is adjacent to the target gene or genes regulated by the GRS. A TF complex comprises a protein complex capable of associating with DNA by direct binding, or via other DNA-binding proteins or complexes, and regulating transcription of a target gene or genes. A TF complex may be positioned between a TFB element and a gene modifier element and interactions with those elements to promote or block recruitment of RNA polymerase to a target gene or genes of a GRS. A non-limiting example of a TFC is a PRC2 complex, which may be physically positioned between a TFB element and a gene modifier element. A TFC positioned between a TFB element and a gene modifier element and is not considered to include elements of either the TFB element or the gene modifier element. The term transcription factor may also be referred to in the art as a sequence-specific DNA-binding factor. See for example, Reiter F, Wienerroither S & Stark A. (2017) Curr Opin in Genet Dev 43, 73-81, the content of which is incorporated by reference herein.

As used herein, a “gene modifier element” refers to an element that controls transcription of a gene of interest (also referred to herein as a target gene). In a non-limiting example, a target gene is a specific cancer suppressor gene and a gene modifier element is a promoter that controls transcription of the cancer suppressor gene. See for example, Haberle V. & Stark A. (2018) Nat Rev Mol Cell Biol 19(10): 621-637, the content of which is incorporated by reference herein.

A GRS is a system of physically interacting components that regulates transcription of specific target genes. A first GRS may regulate transcription of one or more target genes and a second GRS may regulate transcription of (1) one or more of the same target gene(s), (2) none of the one or more target gene(s), or (3) one or more target gene(s) other than those regulated by the first GRS. It has now been identified that agents can be used to modulate an activity of a GRS, and thus can be used to alter transcription of target genes whose transcription is regulated by the GRS.

In some embodiments of the invention, modulating an activity of a GRS includes altering an interaction between two or more of (1) a TFB element, (2) a TF complex, and (3) a gene modifier element of the GRS. In some embodiments of the invention an agent targets and interferes with an interaction between a TFB element and a TF complex of a GRS. In certain embodiments of the invention an agent targets and interferes with an interaction between a TF complex and a gene modifier element of a GRS. In some embodiments of the invention an agent targets and interferes with an interaction between two or more components of a TF complex. In each instance, the agent is capable of interfering with the interaction and the interference reduces or eliminates an activity of the GRS.

### Effects of Modulating a GRS

In certain embodiments of methods of the invention a GRS is contacted with an agent capable of modulating an activity of the GRS. Cellular processes involved in cell development, cancer onset, cancer metastases, tissue growth, cell differentiation, cell de-differentiation, and other physiological changes in cells, tissues, and subjects are regulated, at least in part, by a balance between activation and silencing of particular target genes. For example, using an embodiment of a method of the invention, a GRS is identified and the GRS contacted with an agent that alters an activity of the GRS. The change in the activity of the GRS modulates transcription of a target gene of the GRS. In some embodiments of the invention, modulating transcription of a target gene comprises repressing transcription of the target gene. In certain embodiments of the invention, modulating transcription of a target gene comprises de-repressing transcription of the target gene.

An activity of the GRS includes one or more physical interactions between and/or amount: a TFB element, a TF complex, and a gene modifier element in a cell. In some embodiments of the invention, a target gene—the transcription of which is modified by the activity of the GRS—is identified as a condition-inducer gene or a condition-suppressor gene. The term, “condition” as used herein in reference to condition-inducer genes and condition suppressor genes includes genes whose transcription is capable of associated with one or more of: cell differentiation, cell growth, cell de-differentiation, cell division, cell death, a disease, a cancer, a genetic disease, etc. As used herein the term “associated with” means the transcription or lack of transcription of the gene impacts the presence or absence of the condition. Non-limiting examples of condition-inducer and condition-suppressor genes are cancer-inducer and cancer-suppressor genes, respectively. Additional condition-inducing genes and/or condition-suppressor genes to which methods of the invention can be applied are known in the art. It will be understood that the term “condition” as used herein may be a condition of interest to promote or may be a condition of interest to prevent, depending on the nature of the condition. For example, if a condition is a cancer, it is of interest to prevent or reduce the condition in a cell and/or subject, and if a condition is cell differentiation, it may be of interest to promote the condition in a cell and/or subject.

In certain embodiments of methods of the invention, an effect of the activity of a GRS on repression and de-repression of transcription of a target gene is determined. It will be understood that depending on the effect of transcription of a target gene on a condition, it may be desirable to repress or de-repress transcription of the target gene. For example, though not intended to be limiting: if a target gene is a cancer-suppressor gene and the effect of the activity of the GRS is de-repression of the transcription of the target gene, it may be desirable to increase or enhance the activity of the GRS to inhibit (treat) the cancer. In another non-limiting example, if a target gene is a cancer-suppressor gene and the effect of the activity of the GRS is repression of the transcription of the target gene, it may be desirable to reduce the activity of the GRS to inhibit (treat) the cancer. In another non-limiting example if a target gene is a cancer-inducer gene and the effect of the activity of the GRS is repression of the transcription of the target gene, the activity of the GRS inhibits the cancer; it may be desirable to increase or enhance the activity of the GRS to inhibit (treat) the cancer. In yet another non-limiting example, if a target gene is a cancer-inducer gene and the effect of the activity of the GRS is de-repression of the transcription of the target gene, it may be desirable to reduce the activity of the GRS to inhibit (treat) the cancer.

### Identifying Candidate Agents and Selecting Treatments

Certain embodiments of the invention can be used to identify elements of a GRS and an effect of transcription of a target gene modulated by an activity of the GRS. This information can be used in methods of the invention to determine one or more candidate agents that modify an activity of the GRS and a cell or plurality of cells may be contacted with the identified agent to increase or decrease transcription of a condition-inducer gene that is a target gene of the GRS or condition-suppressor gene that is a target gene of the GRS. The term “agent” may be used herein interchangeably with the term “candidate agent”.

Methods of the invention can be used alter a level of transcription of one or more GRS target genes, wherein the altered level of transcription results in the presence, absence, and/or status of a condition in the cell, plurality of cells, and/or a subject comprising the cell or cells.

In some embodiments of the invention, a plurality of cells having a condition, a non-limiting example of which are cancer cells, can used in comparative studies and to test candidate agents capable of modulating one or more interactions of a GRS. In a non-limiting example, two sets of cancer cells may be maintained in the same environment (for example, both in culture or both in a subject) and one set of cells is contacted with a candidate agent. An effect of the contact on an activity of a GRS in the cells is determined and compared to the activity of the GRS in the second set of cells, which has not been contacted with the candidate agent. In some embodiments of the invention one or more cancer cells from such a culture or subject are included in a method of the invention to assess the cells' status with respect to a candidate agent. As used herein the term “cell status” means the status of the cells with respect to a condition, such as those described herein. For example, if a condition is cell differentiation the status of a cell may be determined to be not differentiated or differentiated. It will be understood that cell status will depend on the specific condition being assessed and that art-known means of assessing and determining the status of a cell are suitable for use in conjunction with methods of the invention.

Some embodiments of methods of the invention include selecting an agent to interfere at a preselected interaction in a GRS. For example in some embodiments of the invention, a candidate agent is selected at least in part because it interferes with an interaction between a TFB element and a TF complex of a GRS. In certain embodiments of the invention, a candidate agent is selected at least in part because it interferes with an interaction between two or more TF complex components. In some embodiments of the invention, a candidate agent is selected at least in part because it interferes with an interaction between a TF complex and a gene modifier element.

In some embodiments of the invention, identifying a target gene of a GRS provides information that can be used to aid in selecting a treatment for a subject with condition. In some embodiments, a cell or subject may be screened for a condition, assess to identify the presence or absence of the condition, or may be recognized as developing the condition at a future time. For example, if the condition is a cancer, a subject or cell may be identified as having the cancer or at risk of having the cancer. In another example, information on the age of a cell or subject can provide information on the developmental status of the cell or subject, thus providing information on the status of differentiation of a cell, plurality of cells, and/or subject. The identification of the presence and/or absence of a condition or stage in a cell or subject permits use of one or more methods of the invention to identify an agent with which to treat the condition in the cell and/or subject.

### Conditions

Embodiments of methods of the invention can be used for identifying one or more characteristics of a condition and its regulation by a GRS. In non-limiting examples, certain embodiments of methods of the invention can be used to: (1) identify a GRS that modulates transcription of a target gene associated with the condition; (2) identify one or more interactions in a GRS having a target gene whose transcription is associated with the condition; (3) identify a an agent capable of interfering with one or more interactions in the GRS, the result of which alters regulation of transcription of the GRS target gene associated with the condition; (4) identify an agent that can be used to a condition associated with transcription of a target gene of a GRS; (5) identify a treatment for a cell and/or subject predicted to have, suspected of having, known to have, and/or known to have had a condition; and (6) selected and/or administer a treatment to a cell and/or subject predicted to have, suspected of having, known to have, and/or known to have had a condition. Non-limiting examples of condition associated with transcription of a target gene of a GRS, include: cell differentiation, cell growth, cell de-differentiation, cell division, cell death, a disease, a genetic disease, a cancer.

As used herein, the term “cancer” is used in reference to a malignant neoplasm. Exemplary cancers include, but are not limited to, acoustic neuroma; adenocarcinoma; adrenal gland cancer; anal cancer; angiosarcoma; appendix cancer; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); cervical cancer (e.g., cervical adenocarcinoma); colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal cancer; head and neck cancer (e.g., head and neck squamous cell carcinoma, oral cancer), throat cancer; hematopoietic cancers (e.g., leukemia such as acute lymphocytic leukemia (ALL); lymphoma such as Hodgkin lymphoma (HL) and non-Hodgkin lymphoma (NHL); multiple myeloma (MM); hemangioblastoma; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); malignant mesothelioma; muscle cancer; myeloproliferative disorder (MPD); neuroblastoma; neurofibroma; neuroendocrine cancer; osteosarcoma; ovarian cancer; papillary adenocarcinoma; pancreatic cancer; penile cancer; prostate cancer; rectal cancer; rhabdomyosarcoma; salivary gland cancer; skin cancer; melanoma; small bowel cancer; soft tissue sarcoma; sebaceous gland carcinoma; small intestine cancer; sweat gland carcinoma; synovioma; testicular cancer; thyroid cancer; urethral cancer; vaginal cancer; and vulvar cancer.

A cancer may be a primary cancer or a metastatic cancer, and may be considered an early or late stage cancer, or a cancer stage in a subject may be characterized with one or more cancer staging classifications known and routinely practice in the art. In some aspects of the invention a cancer is a first cancer in a subject and in certain aspects of the invention a cancer may be a relapse or recurrence of a prior cancer. In some instances, an embodiment of a method of the invention may be used to assess a status of a cancer in a subject who has not been treated with a cancer therapeutic. In certain embodiments, a method of the invention is used to identify a GRS in which transcription of a target gene of the GRS is associated with the cancer. In some embodiments of the invention a cell and/or subject is administered an agent of the invention that interferes with an activity of the GRS and as a result modulates transcription of a target gene of the GRS that is associated with the cancer. In some embodiments the subject administered the agent has been or is currently being treated with one or more cancer therapeutics. Non-limiting examples of cancer therapeutics include: surgery, radiotherapy, chemotherapy, immunotherapy, dietary treatment, or other art-known therapeutic approach.

Certain embodiments of the invention include methods to assist in determining and/or selecting one or more therapeutic protocols for a subject. For example, though not intended to be limiting, some embodiments of the invention may be used to assist in selecting a treatment for a cancer or condition in a subject based, at least in part, on an identified activity of a GRS that regulates transcription of a gene whose transcription is associated with the cancer or condition. Identifying a GRS and its target gene associated with a cancer or condition in a subject using an embodiment of a method of the invention, permits selection of one or more therapeutics based on interactions in identified GRS and the effect on transcription of the GRS target gene resulting from interfering with tone or more of the GRS interactions. Methods of the invention may also be used to identify one or more specific GRS interactions, assess an effect on a GRS target gene of interfering with the one or more specific GRS interactions, and to aid in selecting a treatment for a condition associated with the transcription of the target gene.

For example, in a subject with ovarian cancer, interactions between a TFB element and a TF complex are identified as part of a GRS that regulates a cancer-promoter target gene associated with the ovarian cancer, the information can assist in selecting a treatment for the ovarian cancer that comprises administering to the subject an effective amount of an agent to interfere with the interaction TFB element and the TF complex to reduce transcription of the cancer promoter target gene and treat the ovarian cancer. In certain embodiments of the invention methods are provided that permit determining an efficacy of a candidate agent administered to a cancer cell or to a subject having a cancer, suspected of having a cancer, or at increased risk of having a cancer. Following administration of a selected treatment to the subject, a status of the cancer can be determined in the subject and compared to a control to assess the efficacy of the candidate agent in treating the cancer in the subject.

In some embodiments of the invention a candidate agent may be administered in combination with a second therapeutic agent and/or treatment. In some embodiments, a candidate agent is administered in combination with a second cancer therapeutic agent or in combination with another cancer treatment such as but not limited to one or more of: radiotherapy, chemotherapy, surgery, etc., and a time that is before, after, or interspersed with doses or administration of the candidate agent. In some embodiments, a candidate agent of the present invention is administered to a subject undergoing conventional chemotherapy and/or radiotherapy. In some embodiments the cancer therapeutic agent is a chemotherapeutic agent. In some embodiments the cancer therapeutic agent is an immunotherapeutic agent. In some embodiments the cancer therapeutic agent is a radiotherapeutic agent.

In addition to conditions comprising a cancer, methods of the invention can also be used to identify candidate agents to enhance or inhibit other conditions such as, but not limited to: cell differentiation, cell growth, cell de-differentiation, cell division, cell death, a disease, and a genetic disease.

### Cells

It will be understood that a cell included in a method of the invention may be one of a plurality of cells. As used herein the term, “plurality” of cells may mean a population of cells. A plurality of cells may be all of the same type and/or may all have the same condition. As a non-limiting example, a cell may be obtained from a population of liver cells, and other cells obtained from this population of cells will also be liver cells. In some embodiments of the invention, a plurality of cells may be a mixed population of cells, meaning all cell are not of the same type. In another non-limiting example, a cell may be a cancer cell obtained from a plurality of cancer cells. In another non-limiting example, a cell may be a cell obtained from a plurality of cells at a preselected age or developmental stage. A cell used in an embodiment of a method of the invention may be one or more of: a single cell, an isolated cell, a cell that is one of a plurality of cells, a cell that is one in a network of two or more interconnected cells, a cell that is one of two or more cells that are in physical contact with each other, etc.

In some aspects of the invention a cell may be in a subject; may be obtained from a living animal, e.g., a mammal, a vertebrate, an invertebrate; or may be an isolated cell. An isolated cell may be a primary cell, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation in culture (immortalized cells). In some embodiments of the invention, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc. Cells may be obtained from any organ or tissue of interest, including but not limited to: skin, lung, cartilage, brain, breast, blood, blood vessel (e.g., artery or vein), fat, pancreas, liver, muscle, gastrointestinal tract, heart, bladder, kidney, urethra, and prostate gland. In some embodiments of the invention a cell is a HF-3035 cell, or an HF-2354 cell.

In some embodiments, a cell used in conjunction with the invention may be a healthy normal cell, which is not known to have a disease, disorder or abnormal condition. In some embodiments a cell is a normal cell known to have a condition (for example to be at a particular developmental stage, etc. that is not an abnormal condition). In some embodiments, a cell used in conjunction with methods and compositions of the invention is an abnormal cell, for example, a cell obtained from a subject diagnosed as having a disorder, disease, or abnormal condition, including, but not limited to a degenerative cell, a neurological disease-bearing cell, a cell model of a disease or condition, an injured cell, etc. In some embodiments of the invention, a cell may be a control cell. In some aspects of the invention a host cell can be a model cell for a condition.

A cell that may be used in certain embodiments of the invention is a human cell. Non-limiting examples of a cell that may be used in an embodiment of a method of the invention are one or more of: eukaryotic cells, vertebrate cells, which in some embodiments of the invention may be mammalian cells. Non-limiting examples of cells that may be used in methods of the invention are: vertebrate cells, invertebrate cells, and non-human primate cells. Additional, non-limiting examples of cells that may be used in an embodiment of a method of the invention are one or more of: rodent cells, dog cells, cat cells, avian cells, fish cells, cells obtained from a wild animal, cells obtained from a domesticated animal, and other suitable cell of interest. In some embodiments a cell is an embryonic cell, an embryonic stem cell, or embryonic stem cell-like cell. In some embodiments the cell is a neuronal cell, a glial cell, or other type of central nervous system (CNS) or peripheral nervous system (PNS) cell. In some embodiments of the invention a cell is a natural cell and in certain embodiments of the invention a cell is an engineered cell.

Cells that may be used in embodiments of methods of the invention may be maintained in cell culture following their isolation. Cells may be genetically modified or not genetically modified in various embodiments of the invention. Cells may be obtained from normal or diseased tissue. In some embodiments, cells are obtained from a donor, and their state or type is modified ex vivo using a method of the invention. In certain embodiments of the invention a cell may be a free cell in culture, a free cell obtained from a subject, a cell obtained in a solid biopsy from a subject, organ, or solid culture, etc.

A population or plurality of isolated cells in any embodiment of the invention may be composed mainly or essentially entirely of a particular cell type, cells in a particular state, cells in a specific developmental stage, cells of a specific age, etc. In some embodiments, an isolated population of cells consists of at least 5%, 10%, 20%, 30%, 40%, 50%, 60%, 70%, 80%, 90%, 95%, 96%, 97%, 98%, 99%, or 100% cells of a particular type or state (i.e., the population is at least 5%, 10%, 20%, 30%, 40%, 50%, 60%, 70%, 80%, 90%, 95%, 96%, 97%, 98%, 99%, or 100% pure), e.g., as determined by expression of one or more markers or any other suitable method.

### Controls

Certain embodiments of methods of the invention used to identify a candidate agent for modulating a condition, selecting a candidate agent to treat a condition, and treating a subject with a candidate agent to treat a condition. Methods of the invention, in some embodiments, comprise assessing transcription of GRS target gene(s) in cells, tissue, and/or subjects to determine one or more interactions between or among one or more of a TFB element, a TF complex, and a gene modifier element. Such assessments may comprise comparing results obtained in a sample cell, tissue, or subject with results obtained in a control cell tissue, or subject respectively. As a non-limiting example, some embodiments of the invention include determining an activity of a GRS in a sample cell and in a control cell, wherein the sample sall and control cell have a condition, and comparing the results as a measure of the difference in status of the sample cell and the control cell. In another non-limiting example, a status of an interaction in a GRS having a target gene associated with a condition is identified in a subject having the condition, the subject is subsequently administered a candidate agent intended to alter the identified interaction. The status of the subject can be determined before and after administration of the candidate and any change identified. It will be understood that results obtained from the subject not yet contacted with the candidate agent may be referred to as “control results” and the non-contacted subject as “a control subject”.

As used herein a control may be as described above and also may be a predetermined value, which can take a variety of forms. It can be a single cut-off value, such as a median or mean. It can be established based upon comparative groups. Other examples of comparative groups may include cells or subjects that have a specific condition and cells or subjects without the specific condition. Another comparative group may be a subject from a group with a family history of a condition and a subject from a group without such a family history. Another comparative group may comprise a cell at a specific age or developmental stage and cell at a different age or developmental stage. A predetermined value can be arranged, for example, where a tested population is divided equally (or unequally) into groups based on results of testing. Those skilled in the art are able to select appropriate control groups and values for use in comparative methods of the invention.

Candidate agent identification methods of the invention may be carried out in a cell or cells that are in a subject or in cultured or in vitro host cells. Candidate agent identification methods of the invention that are performed in a subject may include administration of a candidate agent to the subject, wherein the candidate agent is intended to disrupt an activity of a GRS in a cell in the subject. One or more interactions of the GRS may be determined, for example though not intended to be limiting by assessing the interactions themselves and/or by determining a transcription level of a target gene of the GRS, before and/or after administering the candidate agent. A result of contacting a cell, tissue, and/or subject with a candidate agent can be measured and compared to a control value as a determination of an efficacy of the candidate agent in disrupting regulation of a target gene by a GRS.

### Compositions

A composition used in a method of the invention can but need not be a pharmaceutical composition. The term “pharmaceutical composition” as used herein, means a composition that comprises at least one pharmaceutically acceptable carrier that is useful in preparing a pharmaceutical composition that is generally safe, non-toxic, and neither biologically nor otherwise undesirable. A pharmaceutical composition may be used in certain embodiments of methods of the invention, a non-limiting example of which is for administering a candidate agent to a cell or subject to disrupt an activity of a GRS and alter regulation of a target gene of the GRS.

In certain aspects of the invention a pharmaceutical composition comprises one or more candidate agents, with one or more additional molecules, therapeutic agents, candidate agents, candidate treatments, and therapeutic regimens that are also administered to a cell and/or subject. A pharmaceutical composition used in an embodiment of a method of the invention may include an effective amount of a candidate agent to do one or more of: reduce an activity of a GRS; alter transcription of a target gene of the GRS in a cell and/or subject; alter a status of a condition in a cell and/or subject, etc. In some embodiments of the invention, a pharmaceutical composition of the invention may include a pharmaceutically acceptable carrier.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc.

Delivery of a candidate agent to a cell or a subject may be achieved by various means described herein and other art-known means. Such administration may be done once, or a plurality of times. If administered multiple times to a subject, one or more therapeutic agents may be administered via a single or by different routes. For example, though not intended to be limiting: a first (or the first few) administrations may be made directly into a tissue in the subject to be treated, and later administrations may be systemic.

The amount of a candidate agent delivered to a cell or subject may, in certain embodiments of the invention, be an amount that statistically significantly reduces an interaction in a GRS and alters a level of transcription of a target gene of a GRS. Suitable amounts can be readily determined by a practitioner using teaching provided herein in conjunction with art-known methods, for example clinical trials, and without a need for undue experimentation.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Methods for Examples 2-4

### Cell Culture

Mouse embryonic stem cells (ESC) E14 were cultured under feeder-free conditions on 0.1% gelatin coated dishes in Knockout DMEM (Life Technologies) supplemented with 15% FBS (Hyclone), 2 mM L-glutamine (Life Technologies), 0.1 mM non-essential amino acids (Life Technologies), 100 U/ml Penicillin/Streptomycin (Life Technologies), 0.05 mM 2-mercaptoethanol (Sigma), 1,000 U/ml ESGRO mouse LIF Medium Supplement (Leukemia Inhibitory Factor) (Millipore) and maintained at 37° C. with 5% CO2. Cells were fed daily. Primary wild type WT B6 Neo and CRISPR knockout ES cell lines were cultured on irradiated mouse embryonic fibroblast (MEF) feeder layer with high glucose DMEM (Sigma) supplemented with 15% ES Cell FBS (Gibco), 1×non-essential amino acids (Gibco), 1×Glutamax (Thermo Fisher), 100 U/ml Penicillin/Streptomycin (Gibco), 1 mM sodium pyruvate (Gibco), 0.1 mM 2-mercaptoethanol (Gibco) and further supplemented with PD0325901 and CHIR99021 (Selleckchem). Cells were cultured onto feeder-free 0.1% gelatin coated dishes in Knockout DMEM (Life Technologies) prior to harvest.

### Chromatin Immunoprecipitation (ChIP)

Cells were harvested with trypsin (Invitrogen) and suspended in KO-DMEM (Gibco). Cross-linking was performed with 1.5 mM EGS (ethylene glycolbis succinimidylsuccinate) (Sigma) for 45 min followed by 1% formaldehyde for 10 min at room temperature with constant shaking. The reaction was quenched with 0.2M Glycine (Sigma). The cells were washed with PBS (Ambion) supplemented with Protease Inhibitor (Roche). Cells were lysed twice in Lysis buffer (0.1% SDS, 50 mM HEPES-KOH pH 7.5, 150 mM NaCl, 1 mM EDTA, 1% Triton X-100, 0.1% sodium deoxycholate) for 15 min in 4° C. and centrifuged at 1000×g for 10 min. The cells were then sonicated (Branson) in Shearing Buffer (1.0% SDS, 50 mM HEPES-KOH pH 7.5, 150 mM NaCl, 1 mM EDTA, 1% Triton X-100, 0.1% sodium deoxycholate) for 3 min. The sheared chromatin was pre-cleared in 50 μl of Protein A and G Dynabeads (Invitrogen). The pre-cleared chromatin was incubated on the antibody containing Dynabeads overnight in 4° C. for immunoprecipitation. Antibodies used are anti-SUZ12 (ab12073, Abcam), anti-EED (ab4469, Abcam), EZH2 (#39875, Active Motif), H3K27me3 (ab6002, Abcam), RNAPII (MMS126R, clone 8WG16, Covance) and CTCF (ab70303, Abcam). The beads were washed three times in Lysis Buffer, one time in High Salt Lysis Buffer (50 mM HEPES-KOH pH 7.5, 350 mM NaCl, 1 mM EDTA, 1% Triton X-100, 0.1% sodium deoxycholate, 0.1% SDS), followed by a wash in Washing Buffer (10 mM HEPES-KOH pH 7.5, 250 mM LiCl, 1 mM EDTA, 0.5% NP-40, 0.5% Sodium deoxycholate) and finally resuspended in TE buffer.

### ChIP-Seq Library Construction

Immunoprecipitated DNA was treated with end-repair, A-tailing, and ligation of Illumina compatible adapters (IDT, Inc) using the KAPA-Illumina library creation kit (KAPA biosystems). The ligated product was amplified with 8 cycles of PCR (KAPA biosystems). Libraries were sequenced on Illumina Miseq, Nextseq and Hiseq platforms.

### ATAC-seq

ATAC-seq was performed on E14 cell lines as previously reported [Buenrostro, J. D., et al., Nat Methods 10, 1213-8 (2013)]. Libraries were sequenced on Illumina Miseq.

### ChIA-PET Library Construction

ChIA-PET library was prepared as previously describe [Fullwood, M. J. et al. Nature 462, 58-64 (2009)] with the following modifications. The proximity ligated chromatin complex was eluted with 1% SDS (Ambion) and de-crosslinked using proteinase K (Invitrogen) and purified using Zymo ChIP DNA Clean & Concentrator (Zymo Research). The purified fragments were tagmented using the Nextera DNA Sample Preparation Kit (Illumina). The linker ligated ChIA-PET constructs were selected using Sera-Mag Speed Beads Streptavidin-Blocked Magnetic Particles (GE Healthcare). The magnetic beads were blocked with yeast tRNA (Ambion) prior to the selection. The streptavidin selected constructs were amplified with 8-10 cycles of PCR and purified using Ampure XP beads (Beckman Coulter). For the primary mES B6NJ and KO cell clones, the library were prepared with an in situ approach where crosslinked cells were lysed and digested with AluI enzyme (NEB). Fragmented DNA ends were A-tailed and ligated with biotinylated linker overnight [Tang, Z. et al. Cell 163, 1611-27 (2015)]. The ligated chromatin were then sheared by sonication and immunoprecipitated with anti-Ezh2 antibody. The immunoprecipitated DNA were subjected to tagmentation, biotin selection and amplification. Libraries were sequenced on Illumina Nextseq, Hiseq and Novaseq platforms.

### RNA-Seq Library Construction

Total RNA was extracted using the RNeasy Mini Kit (Qiagen). RNA-seq libraries were generated using the Illumina Truseq Stranded RNA LT kits. mRNA was purified from of total RNA using magnetic beads containing poly-T oligos. RNA was fragmented using divalent cations and high temperature. The fragmented RNA was reversed transcribed using random hexamers and reverse transcriptase Superscript II (Invitrogen) followed by second strand synthesis. The fragmented cDNA was treated with end repair, A-tailing, adapter ligation and 10 cycles of PCR. Libraries were sequenced on the Illumina Hiseq and Novaseq.

### CRISPR/Cas9 Targeted Knockout Mouse Generation and Characterization

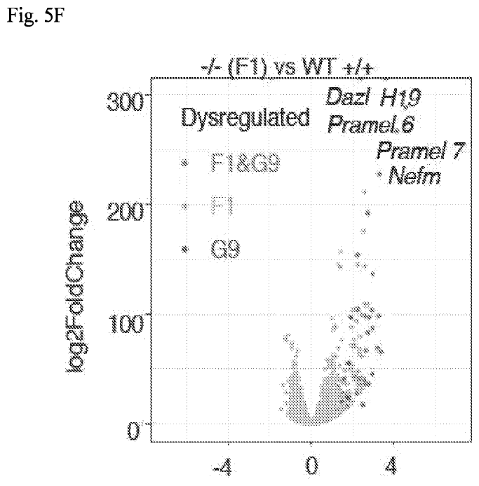

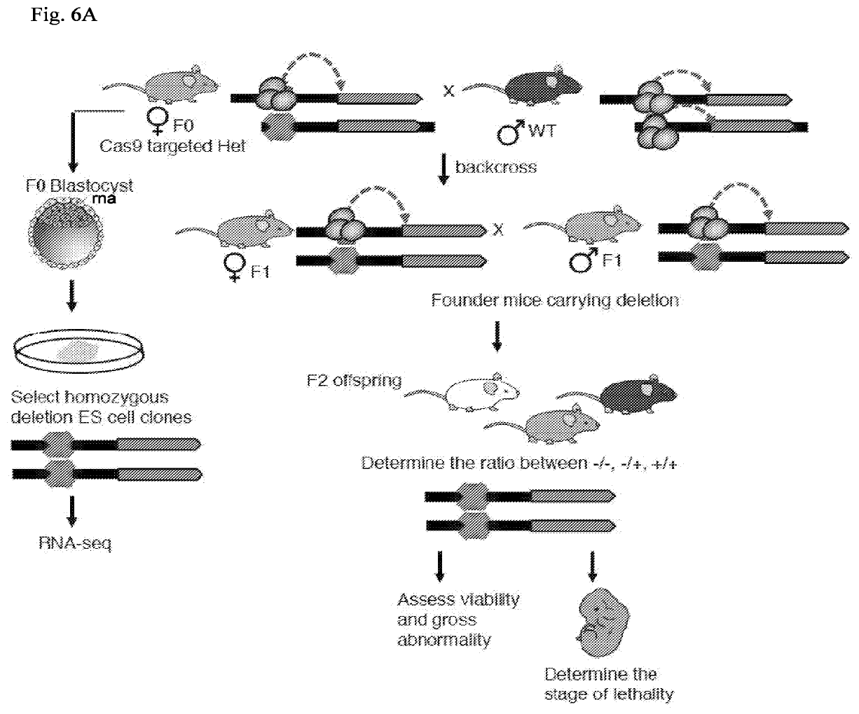

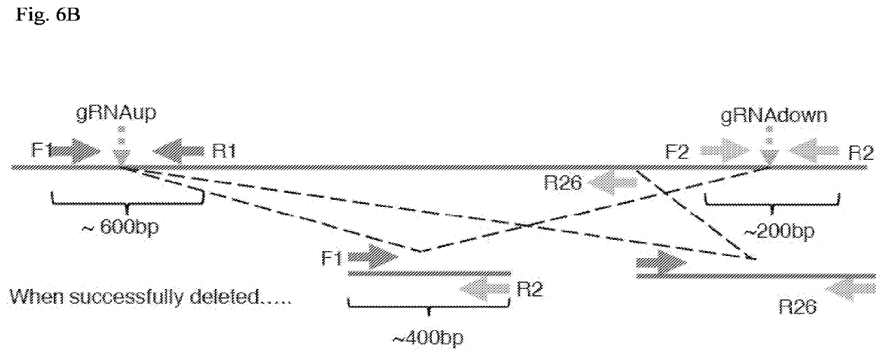

The target regions screened are listed in Table 1. Cas9 RNP including Cas9 protein and sgRNA were electroporated into around 100 C57BL/6NJ mouse zygotes using the ZEN (zygote electroporation of nucleases) technology [Wang, W., et al., Methods Mol Biol 1605, 219-230 (2017)]. After electroporation, 15 zygotes were transferred to a pseudo-pregnant mouse to generate KO mice, 4 transfers for each gene locus. The remaining zygotes were kept in culture in vitro. When these zygotes entered blastocyst stage, they were plated into 96-well plates with MEF feeder cells to generate mouse ES cells. Around 80% of the plated blastocytes successfully generated ES cells. The ES cells were kept in culture, expanded and genotyped for genomic loci KO using the established genotyping strategies. With this method, the brightness of the intermediate band approximately indicate the amount of cells with gene KO. The mouse ES cells with the brightest intermediate band were used for single cell clone screening as described before. Briefly, the ES cells were diluted and plated on a 10 cm dish with MEFs cells as the feeder layer. The single clones grew after plating and were picked and transferred to 96-well plates. The ES cells from single clones were expanded and genotyped using similar strategies (FIG. 6B). To determine the gender of the KO mES cell lines, experiments included a strategy using Rbm3 1x/y method as described previously [Tunster, S. J. Biol Sex Differ 8, 31 (2017)]. The homozygous KO clones were selected for further expansion and cryo-preserved.

For KO mice generation, Founder (F0) mice were genotyped to screen for deletion events by PCR across the cutting sites of expected deleted regions and the confirmed heterozygous female were bred with wild-type male to expand the knockout lines. For embryonic analysis, a single allele from the chr9 line was expanded, timed mating performed, and embryos dissected and examined at embryonic day 9.5 (E9.5). For assessing viability, only viable (visible heartbeat) embryos with unambiguous genotypes were considered.

TABLE 1
Target regions screened identified as #s 1-18, 20, and 21 and primers used with each.

| | | | target | Successful | Successful | | | |
|---|---|---|---|---|---|---|---|---|
| | | | span | KO | ES | Connecting | | |
| # | Actual CRISPR target | | (bp) | mice | clones | genes | Gene name | |
| 1 | chr19: 44741648- | | 10252 | | | 12 | 1700039E22Rik; Pax2; Gm20467; Nkx2- | |
| | 44751900 | | | | | | 3; Hpse2; Kazald1; Lbx1; Lzts2; Pdzd7; Tlx1; | |
| | | | | | | | Tlx1os; Wnt8b | |
| 2 | chr11: 119051389- | | 10295 | ✓#1 | | 20 | 2810410L24Rik; Cbx2; Cbx4; Cbx8; Gm11754; | |
| | 119061684 | | | | | | Gm26508; Ccdc40; Tbc1d16; Gm11762; | |
| | | | | | | | Nptx1; Gm26888; Gm29292; Sphk1; Rnf213; | |
| | | | | | | | Myh4; Otx1; Rbfox3; Wnt3; Hoxb3; Hoxb3os | |
| 3 | chr2: 18603995- | | 10465 | | | 13 | A930004D18Rik; Gm13330; Alx4; Bmi1; | |
| | 18614460 | | | | | | Commd3; Carlr; Gm13344; Gm45314; Nebl; | |
| | | | | | | | Ptf1a; Ptf1aos; Spag6; Skida1 | |
| 4 | chr5: 28113078- | | 10223 | ✓#2 | | 5 | 9530036O11Rik; Gm26894; Shh; En2; Mnx1 | |
| | 28123301 | | | | | | | |
| 5 | chr9: 37451506- | | 10215 | ✓#3 | ✓ | 13 | B230323A14Rik; Foxb1; Esam; Msantd2; | |
| | 37461721 | | | | | | Nrgn; Siae; Vsig2; Pknox2; Robo3; Gm10715; | |
| | | | | | | | Gm10717; Gm17535; Gm26870 | |
| 6 | chr5: 67225652- | | 10638 | | | 8 | Shisa3; Hmx1; 4930458D05Rik; Gpat3; | |
| | 67236290 | | | | | | Limch1; Nkx3-2; Phox2b; Slc5a1 | |
| 7 | chr8: 92455488- | | 10714 | | | 12 | AV026068; Gm28063; Hand2; Crnde; Irx5; | |
| | 92466202 | | | | | | Gm45450; Irx3; Irx3os; Gm45505; Irx6; | |
| | | | | | | | Mmp2; Slc6a2 | |
| 8 | chr3: 107577997- | | 10327 | ✓#4 | ✓ | 10 | AI504432; Gm27008; Kcna3; Alx3; Csf1; | |
| | 107588324 | | | | | | Gm43120; Tbx15; Kcna2; Kcnc4; Slc6a17 | |
| 9 | chr5: 33690271- | | 10445 | | | 6 | Fgfr3; Nat8l; Gm43851; Nkx1-1; Nkx6- | |
| | 33700716 | | | | | | 1; Nsd2 | |
| 10 | chr5: 147216884- | | 10404 | | | 8 | Cdx2; Flt3; Gm26597; Gsx1; Pdx1; Plut; | |
| | 147227288 | | | | | | Uncx; Urad | |
| 11 | chr7: 44423075- | | 10478 | | | 4 | Aspdh; Lrrc4b; Shank1; Clec11 a | |
| | 44433553 | | | | | | | |
| 12 | chr7: 143440438- | | 10278 | ✓#5 | ✓ | 10 | Ascl2; Cdkn1c; Gm44732; Igf2; Igf2os; Kcnq1; | |
| | 143450716 | | | | | | Phhkla2; Asphd1; Gm21984; Sez6l2 | |
| 13 | chr9: 89874601- | | 10369 | | | | Adamts7; B230323A14Rik; Foxb1; Gm17226; | |
| | 89884970 | | | | | | Isl2; Gm29478; Zic4; Rasgrf1; Zic1; | |
| | | | | | | | 4930524O08Rik | |
| 14 | chr15: 103082922- | | 10448 | | | 14 | D930007P13Rik; Gm10830; Gm28265; | |
| | 103093370 | | | | | | Gm28876; Hoxc10; Hoxc11; Hoxc12; Hoxc13; | |
| | | | | | | | Hoxc4; Hoxc8; Hoxc9; Hotair; Hoxc5; Hoxc6 | |
| 15 | chr15: 103056055- | | 10170 | | | 15 | Fignl2; Gm10830; Gm28265; Gm28876; Hoxc10; | |
| | 103066225 | | | | | | Hoxc12; Hoxc13; Hoxc4; Hoxc6; Hoxc8; | |
| | | | | | | | Hoxc9; Krt77; Plec; Hoxc11; Hoxc5 | |
| 16 | chr1: 78213447- | | 10179 | | | 17 | Asic4; BC035947; Mogat1; Cdk5r2; Gm16582; | |
| | 78223626 | | | | | | Dock10; En1; Erbb4; Gm16076; Fev; lhh; | |
| | | | | | | | Hlx; Pantr1; Pax3; Sgpp2; Speg; Tfap2b | |
| 17 | chr5: 119656273- | | 10308 | | | 12 | 1700021F13Rik; Gm16063; Tbx3os1; Gm16064; | |
| | 119666581 | | | | | | Tbx3; Tbx3os2; Gm27199; Lhx5; Tesc; | |
| | | | | | | | Gm43050; Tbx5; Hrk | |
| 18 | chr15: 99092298- | | 4273 | | | 14 | Adcy6; Aqp2; B130046B21Rik; Ddn; C1ql4; | |
| | 99096571 | | | | | | Dnajc22; Cacnb3; Dhh; Kcnh3; Prph; Wnt1; | |
| | | | | | | | Wnt10b; Troap; Tuba1c | |
| 20 | chr2: 18690135- | | 5418 | ✓#6 | ✓ | 18 | 1810010K12Rik; 4930426L09Rik; Pip4k2a; | |
| | 18695553 | | | | | | A930004D18Rik; Gm13330; Bmi1; Commd3; | |
| | | | | | | | Spag6; C130021l20Rik; Lmx1b; Carlr; | |
| | | | | | | | Gm13344; Lhx2; Nr5a1; Nr5a1os; Ptf1a; | |
| | | | | | | | Ptf1aos; Skida1 | |
| 21 | chr19: 45221437- | | 8348 | | | 15 | Atrnl1; Fgf8; Gm15491; Npm3; Gm20467; | |
| | 45229785 | | | | | | Nkx2-3; | |
| | | | | | | | Gm29595; Kazald1; Lbx1; Pax2; Pdzd7; | |
| | | | | | | | Sfxn3; Psd; Tlx1; Tlx1os | |
| | SEQ | | SEQ | | SEQ | | | |
| | ID | | ID | | ID | | | |
| # | NO | gRNAup | NO | gRNAdown | NO | PCR primer F1 | | |
| 1 | 2 | TTTCGCCGCGGAAGC | 22 | GCTCGGCGCTAGTA | 42 | ACGATGCTAATTACGGGGT | | |
| | | AGATT | | CTATTC | | GC | | |
| 2 | 3 | GGCTCTCACAGGTATA | 23 | GTCCATGGGATTCT | 43 | GTGTCTGTAGCTCCCATCCC | | |
| | | GACC | | ACTCCT | | T | | |
| 3 | 4 | GTGCTGTCACGCTAAA | 24 | TACCCTAGGCCCTG | 44 | CTCCTTAAACATGCTATGGC | | |
| | | GAAT | | GTGAAC | | ACTGC | | |
| 4 | 5 | GTCATTTGAACATGGC | 25 | CCGTGCGTGTCACA | 45 | GAGAGAGTGATTTCTGGCAT | | |
| | | TACC | | CCGTAC | | GGG | | |
| 5 | 6 | GGACCCTACACAGATA | 26 | AAGTAACTAGGTTG | 46 | CTGGTACCCACCATATACAT | | |
| | | GGCT | | TCTGAT | | AGGACA | | |
| 6 | 7 | TTCCTGAAGTCTGTTG | 27 | CATCCATGGGATGC | 47 | ATGTAACCCTGGCAGAGAT | | |
| | | TCGC | | TATTTA | | CTGC | | |
| 7 | 8 | GACTAAGCCCTCTTTC | 28 | ACTCTGCGTTGAAC | 48 | GGCATGAAGGTGTACATGC | | |
| | | CCCA | | CATTCT | | AGA | | |
| 8 | 9 | GCTGCTGGCCCACAA | 29 | GCAAGCTTATCATA | 49 | AAGGCACAGCTGTATTGCCT | | |
| | | TCTGG | | CAACTG | | G | | |
| 9 | 10 | AGCCACCAGCTTATGC | 30 | GCTCCGGTGTTGAC | 50 | TGACGTTCCCATTTCCTGAC | | |
| | | CCGG | | TTACCT | | CTT | | |
| 10 | 11 | TGTGGTCCCCGCCAT | 31 | GCAGCGTGCGGGG | 51 | CCATATGATCTGGCTCCTTC | | |
| | | AATCC | | AATGAGT | | CAC | | |
| 11 | 12 | GACCCCAGGCACAGT | 32 | CCAATAAGTCACTA | 52 | AGATTCTAGGCAGGAGCTC | | |
| | | CTTAT | | GGGGCG | | TACCA | | |
| 12 | 13 | GTCAACCGTCTTGGCT | 33 | GTGATGCATAAATG | 53 | GGTTGTTGTTGCTGCTGCTA | | |
| | | TCGA | | CAGTGC | | CT | | |
| 13 | 14 | TTAAGTGTCACCGGTA | 34 | GGCTAGACGAGAAC | 54 | TCTCAGATGCTAAGAGGATC | | |
| | | GCCA | | TAGTGA | | CCTC | | |
| 14 | 15 | ACTCTTGCTGACCATC | 35 | TTGGAGAAGGGTAG | 55 | CTTAGGGTTCATCCTGGGTC | | |
| | | ACCG | | TAAGAC | | TCT | | |
| 15 | 16 | CTGCTCCATATCCTTG | 36 | TAGGCCTTTCAGAT | 56 | CTCACTGATAGTGGCCATCA | | |
| | | GAGC | | GATGCC | | CAAG | | |
| 16 | 17 | CAATCTCTGTAGTGAA | 37 | CTCTTCGCCGCACA | 57 | GATGGCACACACCTTTGATT | | |
| | | GGTA | | GTACTC | | CCAG | | |
| 17 | 18 | ATTTGCTATCAGGCTA | 38 | TCAGAGAACTCTTTT | 58 | GACTCAGGCAGTTTACCTG | | |
| | | AGGC | | TGGAC | | GATG | | |
| 18 | 19 | CATCGTATTCTTTTGC | 39 | CCGTCGCTTTTCATT | 58 | GCAGTCAGTGCTCTTAACCA | | |
| | | ATCC | | CCGGG | | CTGA | | |
| 20 | 20 | GCTCAAGGCGCGAAC | 40 | CTAGACCCTTAAGA | 60 | GGAACTGGGAACCTAAACT | | |
| | | ACCCG | | CATACG | | GTGG | | |
| 21 | 21 | AGGACAGCCTGCGTA | 41 | AGCGATCAAGGAAC | | AGCTTAAAGCGGCTACTGC | | |
| | | CATCA | | TCTGGG | | ATG | | |
| | SEQ | | SEQ | | SEQ | | SEQ | |
| | ID | | ID | | ID | | ID | PCR primer |
| # | No | PCR primer R1 | NO | PCR primer F2 | NO: | PCR primer R2 | NO | R26 |
| 1 | 62 | GAGAGGGGAAG | 82 | TTCCTGTAGGCTG | 102 | AAAGGGGCCTCA | | |
| | | GGAGTTATCGT | | TAGGGTGAA | | TACTGGCA | | |
| | | TT | | | | | | |
| 2 | 63 | TTCCACTAGGA | 83 | AGACTCCAGGAC | 103 | CCCTCTTTCCAT | | |
| | | GTGTCCATGGT | | ATTCCCTGAT | | CCAGGAAACTCA | | |
| 3 | 64 | TGGTAGAAAGA | 84 | CCTCAGACCACTG | 104 | GCCAGCAGGAA | | |
| | | AGACAGCCTCA | | GAGCCATTT | | CCAAATCTGTT | | |
| | | GC | | | | | | |
| 4 | 65 | GGCTGTGAGCT | 85 | TTCTATCAAAGAG | 105 | CGAAGTACGAGC | | |
| | | GTAGAGTTATCT | | TCGCCAGGGG | | AAGGTAAGTCG | | |
| | | AGG | | | | | | |
| 5 | 66 | ACATCCTCTGTA | 86 | AAGCCCTCTGTGA | 106 | TTACCATACTAG | 122 | AGGGTGGCA |
| | | GGACTTAGGGG | | TGACCCT | | TCACCCGGCAG | | GACTGTCAC |
| | | A | | | | | | AGTCTC |
| 6 | 67 | TTCACACTGTCA | 87 | TCTCTACTTAGGG | 107 | GCCTGCACAGTT | | |
| | | TTGTCCGCG | | GCAGTGAGAC | | CTGCACTT | | |
| 7 | 68 | TCTGTGCCTCA | 88 | CCTGTTCATGATG | 108 | CTGTCATCTTCC | | |
| | | CTGTTGATGAG | | CATCTCTGTCA | | ACAGACCTGTCA | | |
| | | A | | | | | | |
| 8 | 69 | GGAAGCAGGTT | 89 | AGAGAAGAGCTG | 109 | ATGGCAAGCAAG | | |
| | | AGTCCTGTAGT | | CTGTGCATGT | | CCTCAAACTAG | | |
| | | CC | | | | | | |
| 9 | 70 | AGTTAGCAGCT | 90 | GCTTCCTGATCAG | 110 | ATCTTCCCAAGC | | |
| | | CTCATGAACCC | | ATGGCAACA | | GGTCCACAT | | |
| | | A | | | | | | |
| 10 | 71 | ACATACCACAG | 91 | ACTCTAAGCCATA | 111 | AAGCTGGACTTC | | |
| | | AGGGCATTGGA | | GTTGCCCACC | | TCCCAAGCTT | | |
| | | T | | | | | | |
| 11 | 72 | TGTGGAAAGAG | 92 | TGCTGACTAGAGT | 112 | GCAAAACTCTAC | | |
| | | TGTTGCATCAT | | CTCTCAGGGA | | CTCTGAGCCAC | | |
| | | GG | | | | | | |
| 12 | 73 | TGGCAGAGTGA | 93 | CCACTGATGAAG | 113 | CACAAAATAACA | | |
| | | TACTGCTGCA | | GCTCTTGGATT | | GCAAGCTCCCCT | | |
| 13 | 74 | CTGGGCCATAT | 94 | ATGGACATATGCG | 114 | TGGCTCTCCTCA | | |
| | | CTATTGGTAGC | | AAGGGTGAGT | | GAAGCATGAT | | |
| | | CT | | | | | | |
| 14 | 75 | GGTGTAGAAAG | 95 | GGACACATTCAAT | 115 | GGATTGAACCCA | | |
| | | TGACAGACTCC | | CATCCAAATCCCT | | AGGCTTTGAGC | | |
| | | ACA | | | | | | |
| 15 | 76 | ACACAGAGTAT | 96 | CCCACTCTTCCTG | 116 | AGCTCTAAGGCC | | |
| | | GATCCGATCCA | | TTACACTATCTGC | | AGTGGATATACC | | |
| | | CC | | | | | | |
| 16 | 77 | TAGATGCCATTT | 97 | CCGTTACATCTGA | 117 | GACGACTCTGAA | | |
| | | GTAGCTCAGCC | | ACCTGTGTTGA | | TTCTGCTCATTG | | |
| | | | | | | G | | |
| 17 | 78 | CAACTTTGGAG | 98 | GGTGCTAGGGAC | 118 | TGGTGCTTTGAG | | |
| | | ACCAGAGACTG | | AGAAATGGAAC | | GAAGCATCCT | | |
| | | C | | | | | | |
| 18 | 79 | TTCATCCCTCCT | 99 | AGCAGAGGTAGA | 119 | GCTTCCCAATCA | | |
| | | TTTCCTGTACCC | | GCTTCCTTGTTG | | GGAATATCGACC | | |
| 20 | 80 | CAGATACAGAA | 100 | GGAATAGGGAAA | 120 | TGAGCACACCAT | | |
| | | GCTCAATCCCG | | GCCATGTGTTGG | | TCAGTTGTGC | | |
| | | AAG | | | | | | |
| 21 | 81 | CATCCTGAAGG | 101 | AGGAACCGAAAG | 121 | GAACTCCAGGTT | | |
| | | GCATGAGGGAT | | TGGTCTGCA | | TCACCGAAGAG | | |
| | | A | | | | | | |

### Phenotyping Screening

Systematic mouse phenotyping screen was conducted for five homozygous silencer KO strains using a broad-based phenotyping pipeline [Dickinson, M. E. et al. Nature 537, 508-514 (2016) and Meehan, T. F. et al. The International Mouse Phenotyping Consortium. Nat Genet 49, 1231-1238 (2017)] established by the Knockout Mouse Phenotyping Program (KOMP2) and the International Mouse Phenotyping Consortium (IMPC). The JAX KOMP2 pipeline assessed 14 major domains of 126 traits associated with development, behavior and physiology. To determine the significance of the phenotypes changes, cohorts of at least five age-matched, sex-matched wild-type C57BL6/NJ mice were phenotyped alongside for each test. Statistical analysis was performed using PhenStat R package [Kurbatova, N., et al., PLoS One 10, e0131274 (2015)]. For categorical data including eye and shirpa procedure, Fisher's Exact test was used, while continuous data analysis was performed using Linear Mixed Model framework in PhenStat which uses linear mixed models in which batch (date of test) was included as a random effect and body weight as a covariate. The association of significance were adjusted for multiple testing using the Benjamini-Hochberg procedure [Benjamini, Y. & Hochberg, Y. Journal of the Royal Statistical Society. Series B (Methodological) 57, 289-300 (1995)] to control the FDR at 5%.

### Confirmation of the Deletion Regions in the KO ES Clones

DNA was extracted using All Prep DNA/RNA Mini kit (Qiagen) according to manufacturer's instruction. PCR was performed using with 2×Kapa HiFi HotStart Ready Mix (Kapa Biosystems) with 3 min denaturation at 95° C., followed by 25-30 cycles of 20 s at 98° C., 15 s at 65° C. and 15 s at 72° C. and a final elongation of 1 min at 72° C. Primer sequences used are listed in Table 1.

To detect the deletion events in the target loci, a strategy using two pairs of primers (F1/R1/F2/R2) was used. In the control group, when the two pairs of primers are used in the PCR reaction, the first pair (F1/R1) yields a PCR product around 600 bp and the second pair yields a PCR product around 200 bp. In the electrophoresis analysis, there will be two bands at 600 bp and 200 bp respectively. If a deletion event is generated, primers R1 and F1 lost their binding site. Only F1 and R2 are functional to generate a PCR product around 400 bp in length (FIG. 6B).

### ChIA-PET Data Processing, Interaction Calling and Annotation

ChIA-PET data was processed with ChIA-PET Utilities, a scalable re-implementation of ChIA-PET Tools [Li, G. et al. Genome Biol 11, R22 (2010)] (see code availability). Briefly, sequencing adaptors incorporated during the tagmentation reaction in the library construction process were removed from the paired reads. To distinguish chimeric ligation events from intra-molecular ligation, two types of linker-ligated chromatin fragments (linkers A and B) were used at equal amounts in the proximity ligation [for details see Zhang, Y. et al. Nature 504, 306-310 (2013)]. The paired reads were binned into either intra-molecular (A-A or B-B) or inter-molecular (A-B) PETs based on their linker sequence and only the intra-molecular ligated PETs were analyzed. Tags identified (>=18 bp) were mapped to mouse genome (mm10) using BWA alignment [Li, H. & Durbin, R. Bioinformatics 25, 1754-60 (2009)] and mem [arXiv:1303.3997 [q-bio.GN], //arxiv.org/abs/1303.3997] according to their tag length. The duplicated pair-end tags arising from clonal PCR amplification were filtered and the uniquely mapped, non-redundant PETs were calssified as inter-chromosomal (L tags and R tags mapped onto different chromosomes), intra-chromosomal (L tags and R tags mapped onto the same chromosome with genomic distance >8 Kb) and self-ligation PETs (L tags and R tags mapped onto the genome <8 Kb). Multiple intra-chromosomal PETs whose respective ends found within 1 Kb were then clustered as iPET-2, 3 . . . . The 1 Kb distance was chosen because of the broader PRC2 binding profile in distance. Further statistical assessment of the PET clusters interaction significance was performed using ChiaSigScaled (see code availability), a scalable re-implementation of ChiaSig [Paulsen, J., et al., Nucleic Acids Res 42, e143 (2014)]. Interaction clusters with member size 3 and above (iPET 3+) and FDR<0.05 were reported.

In the process of constructing ChIA-PET libraries, Tn5 transposon mediated tagmentation was applied to generate chromatin fragments with compatible ends for Illumina library adaptors. Tn5 has known tagmentation bias towards certain sequence context, which resulted in excess sequence coverage and false-positive interaction calls at specific genomic locations. These regions can be defined by their exceptionally high level of ChIP enrichment defined by reads from ChIA-PET sequences regardless of the protein factors used. To distinguish these regions, peak calling was applied onto approximate 600 million reads collected from multiple ChIA-PET libraries using MACS2 (see Binding peak calling in ChIP-seq analysis) and normalized by ChIP-seq input data, to derive 52,964 peak regions with fold-enrichment ranging between 1.2 to 12144 (median: 2.3; 99^{th} percentile: 22.7). Based on the distribution of the fold-enrichment scores, 53 most enriched regions (minimum fold-enrichment is 115, top 0.1% ranked in enrichment scores) were defined followed by visual inspection of their read coverage profiles across multiple tagmentation based ChIA-PET libraries. These regions (See Table 2) were treated as the blacklist regions and used to remove any interactions with whose anchors overlapped from further analysis.

TABLE 2
Fifty three most enriched regions, Numbers 1-53, with
chromosomal (Chr) location included start (Chr start) and
end (Chr end). Fold enrichment results shown in final column.

| | | | | Fold |
|---|---|---|---|---|
| Number | Chr | ChrStart | ChrEnd | enrichment |
| 1 | chr2 | 98,666,024 | 98,667,551 | 12144.41 |
| 2 | chr8 | 25,227,103 | 25,227,590 | 11375.78 |
| 3 | chr2 | 98,662,003 | 98,663,209 | 3356.67 |
| 4 | chr9 | 3,021,878 | 3,028,024 | 1901.89 |
| 5 | chr6 | 103,648,861 | 103,649,501 | 1646.09 |
| 6 | chr2 | 98,664,665 | 98,665,405 | 1618.29 |
| 7 | chr12 | 3,109,648 | 3,110,338 | 1386.80 |
| 8 | chr9 | 35,304,988 | 35,305,818 | 1156.19 |
| 9 | chrX | 143,482,782 | 143,483,325 | 935.76 |
| 10 | chr2 | 61,265,121 | 61,265,604 | 911.77 |
| 11 | chr9 | 2,999,794 | 3,006,174 | 745.21 |
| 12 | chr10 | 60,445,607 | 60,446,088 | 733.63 |
| 13 | chr9 | 3,006,314 | 3,007,746 | 633.36 |
| 14 | chrX | 129,348,231 | 129,348,497 | 541.41 |
| 15 | chr14 | 45,812,598 | 45,813,065 | 539.53 |
| 16 | chr12 | 79,729,401 | 79,729,882 | 501.03 |
| 17 | chr18 | 51,252,579 | 51,252,832 | 476.15 |
| 18 | chr11 | 74,700,383 | 74,700,868 | 454.40 |
| 19 | chr14 | 19,415,533 | 19,419,929 | 430.53 |
| 20 | chr13 | 104,999,507 | 104,999,987 | 367.32 |
| 21 | chr11 | 34,085,202 | 34,085,463 | 365.04 |
| 22 | chr4 | 57,768,187 | 57,768,440 | 321.74 |
| 23 | chr2 | 179,753,826 | 179,754,308 | 317.05 |
| 24 | chr16 | 93,685,746 | 93,687,407 | 311.99 |
| 25 | chr2 | 123,634,730 | 123,634,989 | 262.27 |
| 26 | chr7 | 90,441,768 | 90,442,225 | 246.70 |
| 27 | chrUn_GL456389 | 7,936 | 14,143 | 235.83 |
| 28 | chr10 | 8,649,716 | 8,650,194 | 222.84 |
| 29 | chr2 | 158,316,180 | 158,317,622 | 221.49 |
| 30 | chr17 | 93,046,209 | 93,046,741 | 219.62 |
| 31 | chr9 | 3,028,556 | 3,029,026 | 213.83 |
| 32 | chrUn_JH584304 | 87,803 | 88,520 | 211.69 |
| 33 | chrUn_JH584304 | 103,306 | 104,047 | 200.95 |
| 34 | chrUn_GL456392 | 14,421 | 23,718 | 191.95 |
| 35 | chr10 | 121,763,017 | 121,763,489 | 188.31 |
| 36 | chr2 | 173,268,039 | 173,268,464 | 186.51 |
| 37 | chr4 | 79,115,927 | 79,116,405 | 162.63 |
| 38 | chrUn_GL456396 | 8,118 | 15,827 | 158.50 |
| 39 | chr8 | 98,154,789 | 98,155,039 | 150.52 |
| 40 | chr3 | 74,382,342 | 74,382,602 | 144.98 |
| 41 | chr19 | 12,050,100 | 12,050,358 | 144.06 |
| 42 | chr1 | 129,209,097 | 129,209,572 | 139.94 |
| 43 | chrUn_JH584304 | 72,619 | 73,538 | 138.91 |
| 44 | chr9 | 3,015,051 | 3,016,561 | 130.27 |
| 45 | chr17 | 72,058,977 | 72,059,256 | 129.16 |
| 46 | chr5 | 133,533,159 | 133,533,412 | 124.00 |
| 47 | chr9 | 24,541,804 | 24,542,376 | 123.59 |
| 48 | chr17 | 67,741,184 | 67,741,803 | 122.74 |
| 49 | chr9 | 3,018,126 | 3,020,756 | 116.76 |
| 50 | chr14 | 93,623,941 | 93,624,494 | 116.13 |
| 51 | chr3 | 76,527,193 | 76,527,450 | 115.41 |
| 52 | chr10 | 72,297,019 | 72,297,568 | 115.36 |
| 53 | chr9 | 3,031,653 | 3,032,932 | 115.05 |

Next, the interactions were classified based on their anchors overlapped with gene models in gencode.vm14.grcm38 (accessed date 2017 Oct. 3). Each anchor was annotated with gene that overlapped at 1 bp overlap. To classify each anchor, priority was given to promoter (P) region (defined as ±2.5 kb of TSS) followed by gene region (G). Anchors that do not overlap with any gene or promoter region were classified as intergenic (I). The interaction classification is just the combination of its anchors classification with additional prefix “s” should both anchors overlap common gene(s).

### ChIP-Seq Data Analysis

Single-end reads were quality trimmed along with the adapter if present using cutadapt (options: -e 0.2 -a AGATCGGAAGAGC (SEQ ID NO: 1) --minimum-length 20 --trim-n -n 3). The trimmed-reads were mapped on the mm10 genome using ‘bwa aln’, and only the reads mapped uniquely onto the genome were collected followed by removing duplicates. Peak calling was performed on ChIP-seq and ChIA-PET reads using MACS2.1.0.20151222 [Liu, T. Methods Mol Biol 1150, 81-95 (2014] with flags --keep-dup all --nomodel --extsize 250 -B -SPMR -g mm. Narrow peaks results were collected for all factors, except H3K27me3 where broad peaks were called. Besides the data generated, ENCODE public data sets were also included. Using the same pipeline, H3K27ac (ENCFF001KFX) and H3K4me1 (ENCFF001KFE) were processed with ENCFF071UWJ as the input control. The procedures included downloading and using the pre-calculated fold-change enrichment data for H3K9me3 (Encode bigWig file ENCFF857TIJ).

### Consistency Between ChIA-PET Biological Replicates

To assess the consistency of ChIA-PET replicates, correlation analysis among replicates was performed. First, interaction matrix with 500 kb binning containing iPET counts was constructed for each library. Blacklist regions were excluded from computation. In addition, a matrix aggregated from all libraries with the same protein factor was calculated to select the bins representing frequent interacting regions. This selection was made to minimize effect of predominant sparse interactions (zero elements in the matrices) when calculating the correlation. The sums of every bin in the aggregate matrix were computed. The 20% bins with the highest counts were selected to be included in correlation computation. Pairwise Pearson's correlation coefficient, r, was computed for libraries within the same protein factor. r values were influenced by the sequencing depth so replicates with lower numbers of sequencing reads generally yielded lower r values.

### RNA-Seq and Differential Gene Expression Analysis of the KO ES Lines

Reads were trimmed using Trim Galore! [//github.com/FelixKrueger/TrimGalore] to remove adapters and low-quality portion of the reads. Trimmed reads were aligned to mm10 genome and gencode.vm14.grcm38 transcripts with hisat2 (version 2.1.0) [Kim, D., et al., Nat Methods 12, 357-60 (2015)]. To ensure accuracy in read-to-transcript assignment, gene models known as not transcribed by RNA polymerase II like pseudogenes, snoRNA, snRNA, miscRNA and riboRNA were removed to yield 30,517 gene models for subsequent RNA-seq analysis. HTSeq [Anders, S., et al., Bioinformatics 31, 166-9 (2015)] was used to quantify the mapped transcripts, with parameters for reverse strandedness (-s=reverse) and assigning reads that are assigned to more than one feature to all aligned featured (−nonunique=all). Using these counts, differential gene expression analysis was performed with R package DESeq2 [Love, M. I., et al., Genome Biol 15, 550 (2014)]. The following analysis was performed for each dataset—si-Δchr9 (N_{si-Δchr9-F1}=2, N_{si-Δchr9-G9}=3) and wildtype (N_{wildtype}=3) or si-Δchr7 (N_{si-Δchr7-F4}=3, N_{si-Δchr7-D4}=3) and wildtype (N_{wildtype}=3)—separately. First, the counts were normalized using DESeq2's built-in count normalization approach (e.g. counts(dds, normalized=TRUE)). Then, differential expression analysis was performed using DESeq2 between knock out and wildtype and applied shrinkage for log 2 fold change estimation using lfcShrink from the DESeq2 package. P-values were adjusted using the method of Benjamini and Hochberg [Benjamini, Y. & Hochberg, Y. Journal of the Royal Statistical Society. Series B (Methodological) 57, 289-300 (1995)]. Genes were considered dysregulated (differentially expressed) when the adjusted p-value <0.05 and either log 2 fold change >2 or log 2 fold change <−2.

### Feature Analysis of PRC2 Bound Distal Regulatory Elements (DRE)

The features explored were open chromatin, TFBS and enhancer frim the ENSEMBL regulatory build [Zerbino, D. R., et al., Genome Biol 16, 56 (2015)], the key pluripotent TFBS [Chen, X. et al. Cell 133, 1106-17 (2008)], and CGI from UCSC Genome Browser [Gardiner-Garden, M. & Frommer, M. J Mol Biol 196, 261-82 (1987)]. Specifically, the fold enrichment is the fraction of observed overlap between the 1800 DREs and the feature against the expected background established from 1000 simulations. For each simulation, the DREs were randomly permuted on the chromosome with shuffleBed disallowing overlapping amongst the permuted DREs, the N regions and blacklist. The number of the shuffled DREs that overlap the features is recorded to establish the average and standard deviation of the expected background. The Z-score/standard score is computed as the signed fractional number of the background standard deviations by which the empirical observations is above the background mean.

### Gene Ontology Analysis

GO enrichment analysis was performed on 5,825 genes that were involved in BA-interactions against the 24,692 genes without BA-interactions using GOrrilla (//cbl-gorilla.cs.technion.ac.il) with “Mus musculus” selected as reference. The set of 5,825 genes was uploaded as the target, and the set of 24,692 genes was uploaded as the background.

### Hi-C Data Processing

The mESC Hi-C data (GSE35156) [Dixon, J. R. et al. Nature 485, 376-80 (2012)] was processed with Juicer tools [Durand, N.C. et al. Cell Syst 3, 99-101 (2016)] and produced the hic file. Using HiTC (R Bioconductor package) [Servant, N. et al. Bioinformatics 28, 2843-4 (2012)], the AB compartments were then called using gene density data (in R library BSgenome.Mmusculus.UCSC.mm10) by calling ‘pca.hic.regular’ function.

To investigate the interaction between si-Δchr9 to the 29 derepressed genes in the KO clones, the most recent high resolution Hi-C data on mESC (GEO number: GSE96107; all 14 runs of HiC_ES_1 SRX2636666) [Bonev, B. et al. Cell 171, 557-572 e24 (2017)] were interrogated. A total of over 2.5 billion read was processed with Juicer tools [Durand, N.C. et al. Cell Syst 3, 99-101 (2016)]. The processed reads resulted 1.2 billion Hi-C contacts, then an interaction matrix with 100 kb resolution was generated and ICE-normalized for analysis. The bins where those gene loci were located in the interaction frequency (IF) matrix were identified. In this analysis, the only focus was on trans-chromosomal interactions: si-Δchr9 on chr9 with 26 derepressed genes on different chromosomes (3 genes on chr9 were excluded). The average trans-chromosomal interaction frequencies (TIF) of si-Δchr9 bin to the bins that housed these genes was computed. This value was then compared with random picked genes. The distribution of random bins on different chromosomes followed the distribution of chromosomes in the real case. The random picks were permuted 100,000 times, and the average TIF between si-Δchr9 and random picks was computed for each permutation. It was observed that the average TIF of si-Δchr9 to the derepressed genes was significantly higher than that of the random picks (Wilcoxon tests' p-values <2.2E-16). Another background model that was tested comprised of all bins that contained genes but excluding bins where repressed genes resided. The random permutation was repeated 100,000 times, and it was concluded that the average TIF between si-Δchr9 and derepressed genes was significantly higher than that of random background (Wilcoxon test p-values <2.2E-16).

### Example 2

### PRC2 Mediates Extensive Chromatin Interactions to Transcriptionally Silence Developmentally-Regulated Genes

### (For Methods See Example 1)

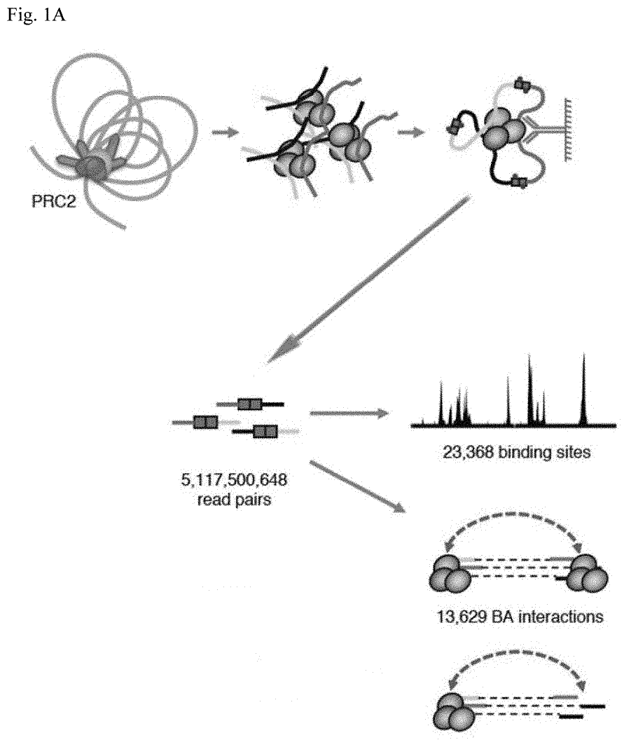

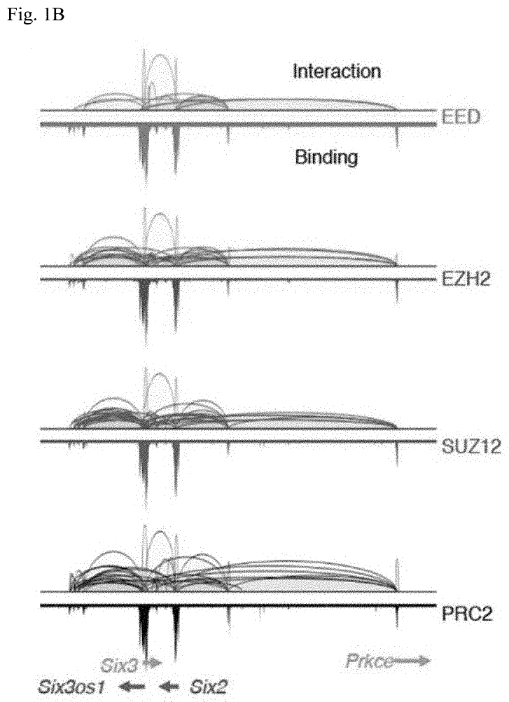

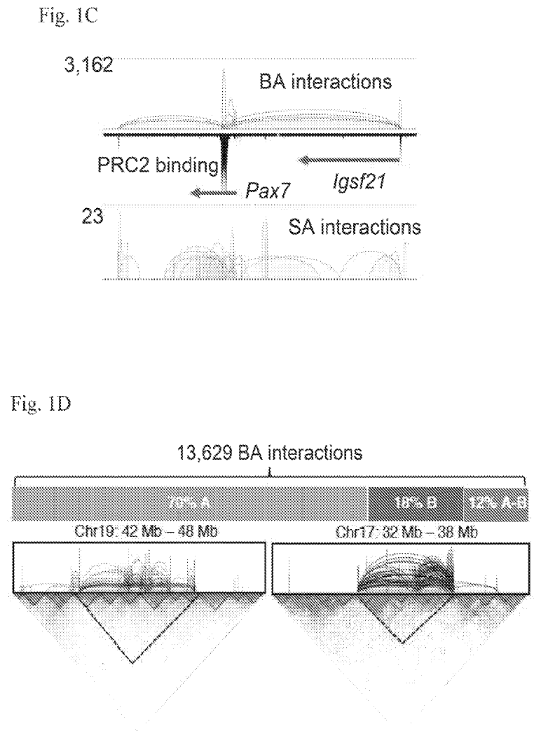

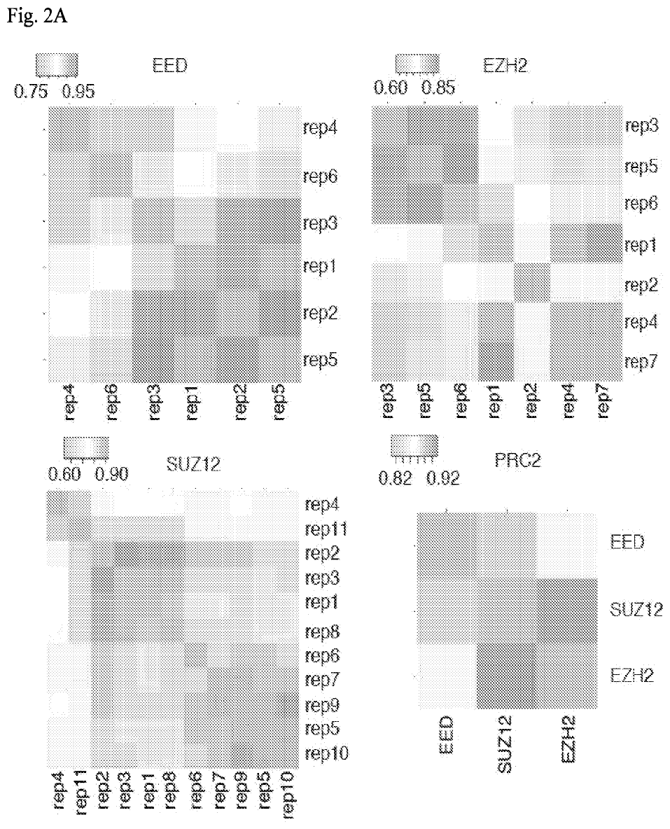

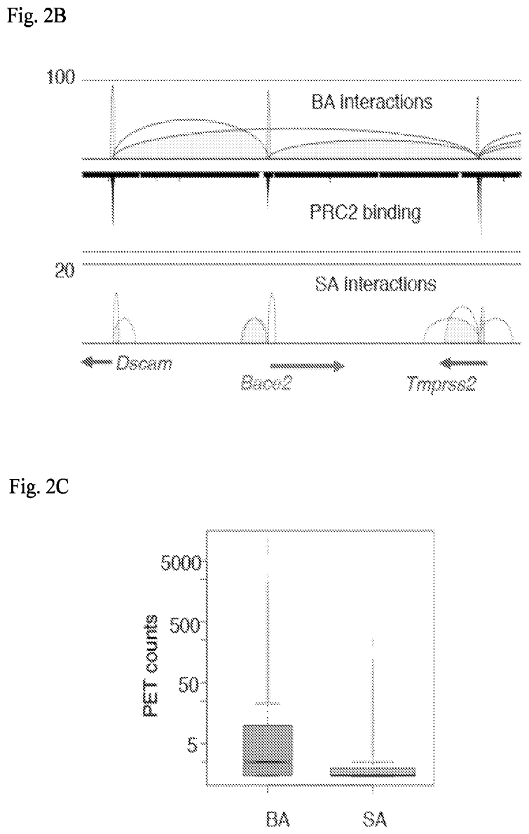

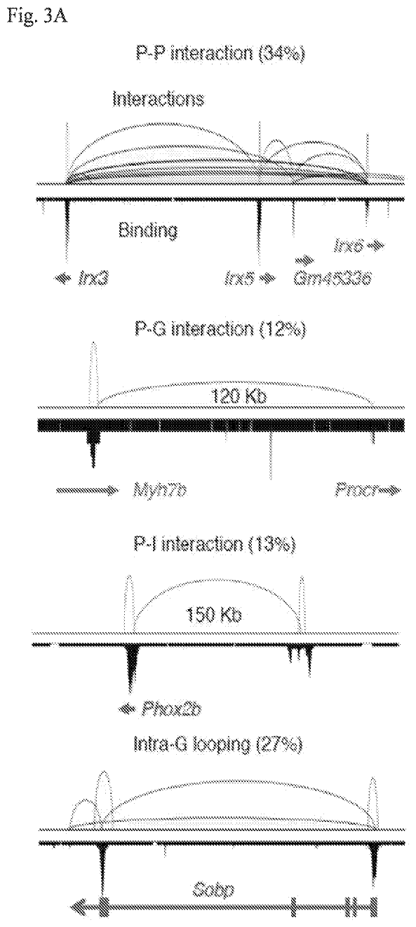

PRC2-mediated chromatin interactions were characterized in mESCs by ChIA-PET analysis. ESC chromatin was crosslinked and genomic regions connected by PRC2 were captured by proximity ligation of crosslinked chromatin followed by ChIP using antibodies against each of the three core subunits of PRC2 complex, namely EED, EZH2 and SUZ12 in mESCs (FIG. 1, Methods). To maximize the sensitivity of the ChIA-PET library approach in capturing PRC2-mediated interactions, multiple biological replicates were constructed, EED (n=6), EZH2 (n=7) and SUZ12 (n=11), and generated a total of 5 billion paired-read sequences (See Table 3). The replicates datasets showed a high degree of consistency (FIG. 2A), and were therefore merged to define 25,000-42,000 protein binding sites (FDR <0.05) and 12,000-28,000 significant chromatin interactions (FDR <0.05, p<0.05) supported by EED, EZH2 and SUZ12 binding (See Table 4).

TABLE 3
Paired-read sequence information for EED, EZH2, and SUZ12.

| Factor EED Library ID | Rep1 | | | | Rep2 | | | |
|---|---|---|---|---|---|---|---|---|
| Technical replicate (TR)/Run | TR1 | | TR2 | | 1 | | 2 | |
| Total read pairs | 48,353,975 | | 312,336,001 | | 272,192,222 | | 334,956,195 | |
| 2-tag read pairs | 9,728,506 | 20.12% | 68,550,539 | 21.95% | 82,685,907 | 30.38% | 104,400,893 | 31.17% |
| uniquely mapped | 4,218,290 | 61.03% | 29,184,245 | 59.47% | 53,897,734 | 68.97% | 68,543,142 | 69.25% |
| non-redundant | 4,061,135 | | 24,933,645 | | 39,923,090 | | 49,175,268 | |

| Rep3 | | Rep4 | | Rep5 | | Rep6 | |
|---|---|---|---|---|---|---|---|
| 245,180,304 | | 84,606,548 | | 235,595,520 | | 42,153,806 | |
| 91,242,773 | 37.21% | 29,882,917 | 35.32% | 72,602,993 | 30.82% | 13,542,441 | 32.13% |
| 62,491,782 | 72.12% | 14,881,120 | 73.35% | 48,288,777 | 69.64% | 9,487,937 | 71.81% |
| 49,397,010 | | 13,768,689 | | 42,612,194 | | 8,624,253 | |

| Factor EZH2 Library ID | Rep1 | | | | | | | |
|---|---|---|---|---|---|---|---|---|
| Technical replicate (TR)/Run | TR1 | | TR2 | | TR3 | | Rep2 | |
| Total read pairs | 40,460,349 | | 389,585,403 | | 197,615,507 | | 28,899,730 | |
| 2-tag read pairs | 9,628,197 | 23.80% | 94,220,371 | 24.18% | 46,576,190 | 23.57% | 6,838,587 | 23.66% |
| uniquely mapped | 5,393,093 | 71.74% | 52,215,889 | 70.88% | 26,309,730 | 72.46% | 4,496,424 | 68.27% |
| non-redundant | 5,001,006 | | 32,251,766 | | 19,699,788 | | 3,874,274 | |

| Rep3 | | Rep4 | | Rep5 | | Rep6 | | Rep7 | |
|---|---|---|---|---|---|---|---|---|---|
| 56,410,591 | | 29,909,437 | | 87,799,730 | | 137,955,314 | | 66,117,359 | |
| 14,727,844 | 26.11% | 8,807,114 | 29.45% | 22,860,569 | 26.04% | 46,167,491 | 33.47% | 19,313,306 | 29.21% |
| 9,615,492 | 66.24% | 6,525,349 | 76.44% | 16,007,876 | 71.30% | 33,250,173 | 73.10% | 14,145,204 | 75.51% |
| 7,139,330 | | 5,454,979 | | 11,341,829 | | 15,993,920 | | 11,596,650 | |

| Factor SUZ12 Library ID | | | | | | | | |
|---|---|---|---|---|---|---|---|---|
| Techincal replicate (TR)/Run | Rep1 | | Rep2 | | Rep3 | | Rep4 | |
| Total read pairs | 286,163,589 | | 361,687,345 | | 155,411,949 | | 52,572,966 | |
| 2-tag read pairs | 127,134,751 | 44.43% | 183,083,722 | 50.62% | 76,914,049 | 49.49% | 10,255,918 | 19.51% |
| uniquely mapped | 70,064,222 | 67.90% | 93,891,712 | 68.46% | 41,690,332 | 71.86% | 4,819,668 | 64.03% |
| non-redundant | 21,007,077 | | 84,891,257 | | 38,717,337 | | 4,490,198 | |

| Rep5 | | Rep6 | | Rep7 | | Rep8 | |
|---|---|---|---|---|---|---|---|
| 277,326,566 | | 118,133,687 | | 175,145,114 | | 106,235,993 | |
| 58,087,804 | 20.95% | 39,721,303 | 33.62% | 35,976,589 | 20.54% | 26,717,350 | 25.15% |
| 28,429,166 | 64.19% | 26,532,582 | 68.08% | 19,461,510 | 61.12% | 12,087,513 | 66.36% |
| 21,424,050 | | 10,189,027 | | 14,635,461 | | 9,778,416 | |

| Rep9 | | | | Rep10 | | | | | |
|---|---|---|---|---|---|---|---|---|---|
| 1 | | 2 | | 1 | | 2 | | Rep11 | |
| 89,638,629 | | 443,137,066 | | 75,483,735 | | 331,666,109 | | 34,769,909 | |
| 28,643,845 | 31.95% | 138,130,643 | 31.17% | 21,463,209 | 28.43% | 93,544,913 | 28.20% | 14,037,099 | 40.37% |
| 13,618,767 | 72.86% | 65,045,131 | 71.01% | 10,228,794 | 71.73% | 44,415,248 | 71.55% | 7,730,840 | 76.76% |
| 11,066,096 | | 36,638,956 | | 7,875,645 | | 21,473,499 | | 7,234,025 | |

TABLE 4
Protein binding sites and significant chromatin interactions supported by EED,
EZH2 and SUZ12 binding.

| Factor | EED | | EZH2 | | SUZ12 | | PRC2 (combined) | |
|---|---|---|---|---|---|---|---|---|
| Number of | 6 | | 7 | | 11 | | 24 | |
| replicates | | | | | | | | |
| Total reads pairs | 1,575,374,571 | | 1,034,753,420 | | 2,507,372,657 | | 5,117,500,648 | |
| non-redundant, | 208,931,828 | 13% | 97,936,086 | 9% | 268,046,232 | 11% | 574,914,146 | 11% |
| non-chimeric | | | | | | | | |
| Total binding peaks | 31,366 | | 42,189 | | 25,467 | | 23,368 | |
| Peaks with | 10,789 | 34% | 15,483 | 37% | 11,678 | 46% | 15,428 | 66% |
| interactions | | | | | | | | |
| Interaction clusters | 146,066,534 | | 57,294,283 | | 200,505,313 | | 397,500,613 | |
| Significant cis- | 63,317 | 0.04% | 50,819 | 0.09% | 104,822 | 0.05% | 261,440 | 0.07% |
| interactions | | | | | | | | |
| (FDR < 0.05, | | | | | | | | |
| p < 0.05) | | | | | | | | |
| Interactions with | 12,130 | | 19,302 | | 28,310 | | 54,173 | |
| PRC2 binding | | | | | | | | |
| Interactions with | 7,937 | 13% | 10,718 | 21% | 20,614 | 20% | 40,544 | 16% |
| PRC2 binding | | | | | | | | |
| (single anchor; SA) | | | | | | | | |
| Interactions with | 4,193 | 7% | 8,584 | 17% | 7,696 | 7% | 13,629 | 5% |
| PRC2 binding (both | | | | | | | | |
| anchors; BA) | | | | | | | | |
| Interaction Types | BA | SA | BA | SA | BA | SA | BA | SA |
| Intragenic loops | 1332 | 1799 | 3390 | 3033 | 2380 | 3714 | 3686 | 8925 |
| | (32%) | (23%) | (39%) | (28%) | (31%) | (18%) | (27%) | (22%) |
| promoter anchored | 1915 | 3573 | 3980 | 4717 | 4305 | 11626 | 8,175 | 21,584 |
| | (46%) | (45%) | (46%) | (44%) | (56%) | (56%) | (60%) | (53%) |
| P-P | 951 | 483 | 1,991 | 587 | 2,381 | 1,425 | 4,694 | 2,998 |
| P-I | 593 | 1,820 | 1,248 | 2,541 | 1,060 | 5,791 | 1,800 | 10,710 |
| P-G | 371 | 1,270 | 741 | 1,589 | 864 | 4,410 | 1,681 | 7,876 |
| non-promoter | 946 | 2,565 | 1214 | 2968 | 1011 | 5274 | 1768 | 10035 |
| anchored | (22%) | (32%) | (14%) | (28%) | (13%) | (26%) | (13%) | (25%) |
| I-I | 427 | 1,184 | 669 | 1,459 | 449 | 1,875 | 737 | 4,201 |
| G-G | 189 | 443 | 169 | 478 | 202 | 1,124 | 356 | 1,921 |
| G-I | 330 | 938 | 376 | 1,031 | 360 | 2,275 | 675 | 3,913 |

Consistent with these three subunits functioning together in the PRC2 complex, a high degree of correlation was found among the interactomes mediated by each subunit (r=0.8-0.93, FIG. 2A), as exemplified in a 1 Mb region of the Six2/3-Prkce locus (FIG. 1B). The comprehensive PRC2 interactome was defined by combining all 5 billion read pairs sequences from three subunits to yield 54,173 significant intra-chromosomal interactions (FDR<0.05, p<0.05) which were supported by PRC2 binding at either single anchor (SA) or both anchors (BA) (FIG. 1A, Table 4). BA-interactions have significantly higher numbers of read counts than SA-interactions (Welch two sample t-test, p-value=3.73e-53, FIG. 2B). Based on such feature, BA-interactions (n=13,629) represent specific loops between the PRC2 binding sites, while SA-interactions (n=40,544) are transient and weak connections often found within the BA-interaction regions (FIG. 1C, FIG. 2C). These PRC2-mediated chromatin interactions are largely constrained within topologically associating domains (TADs) [Shin, H. et al. Nucleic Acids Res 44, e70 (2016) and Dixon, J. R. et al. Nature 485, 376-80 (2012)] (FIG. 1D) and also significantly associated with the active, gene-rich A-type compartments than the inactive, gene-poor B-type compartments (9,532, 70%; p-value=1.27e-160). Collectively, these data constitute one of the most extensively surveyed transcriptionally repressive chromatin interactomes.

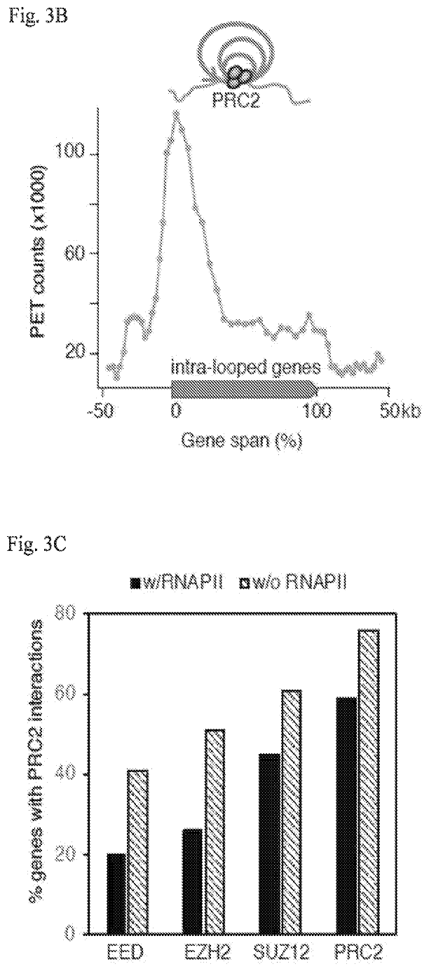

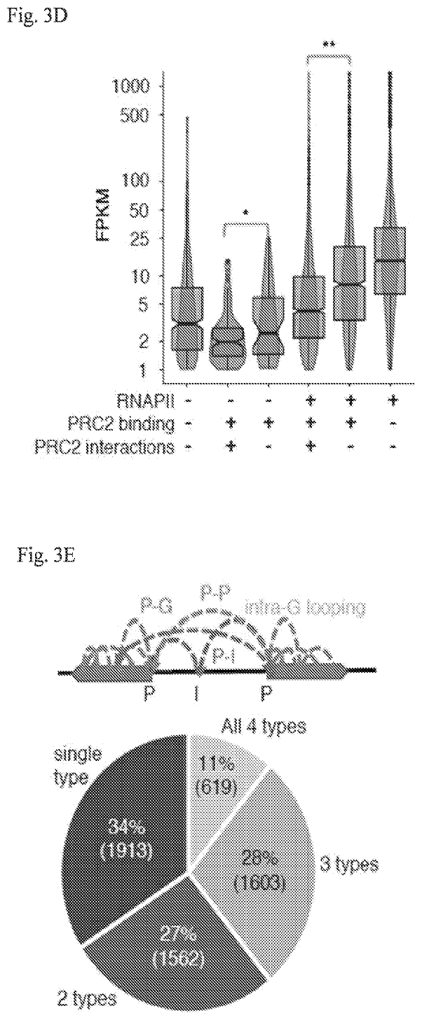

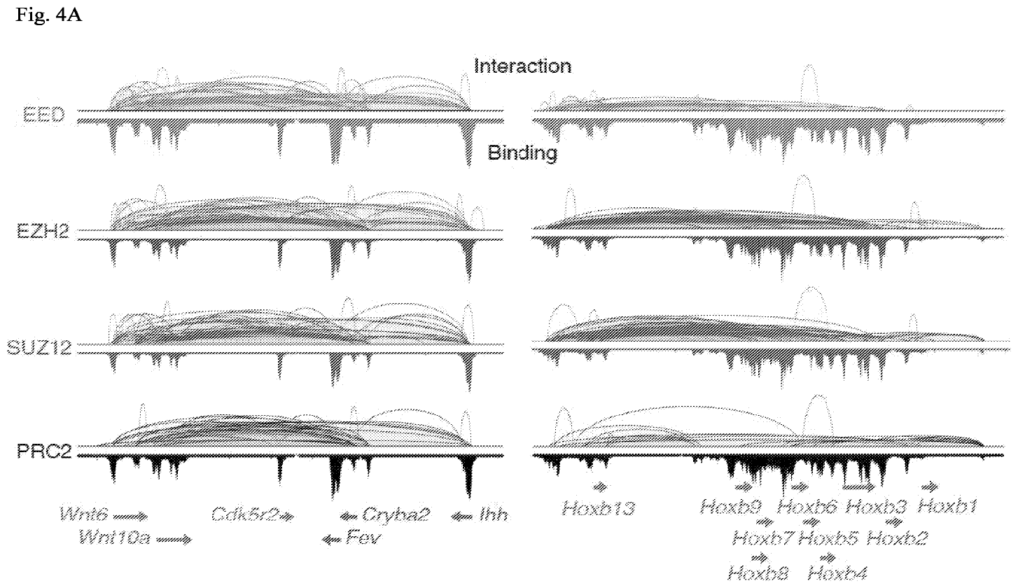

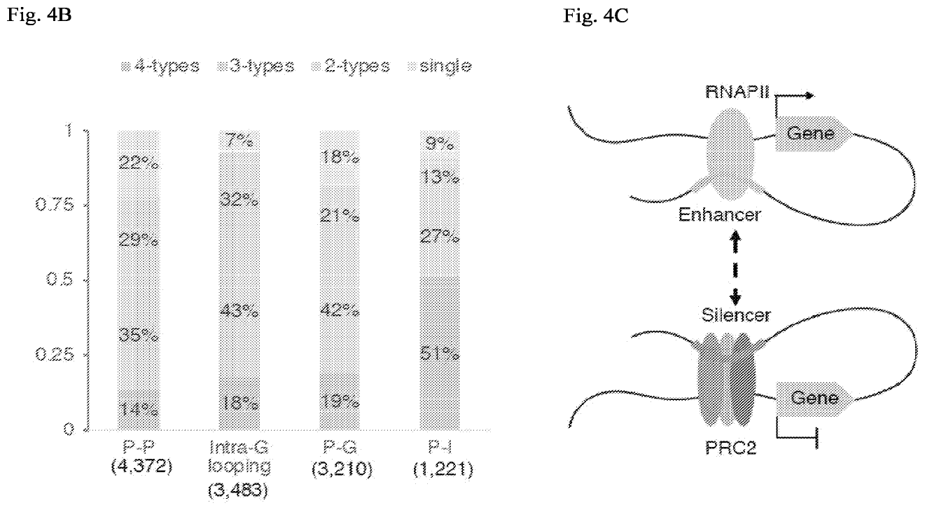

Vast majority (95%) of the 13,629 BA-interactions are found within gene-coding regions and most (60%) of them are anchored at gene promoters (defined as ±2.5 Kb of transcription starting sites (TSS)). These are interactions between promoters (P-P, 34%), or promoter to either intergenic (P-I, 13%) or intragenic regions (P-G, 12%) of distal genes (FIG. 3A). 27% of the BA-interactions are found within individual genes (intra-G), coiling the promoters or looping from 5′ to 3′ of their associated transcription units (FIG. 3A). The distribution of intra-G interaction frequencies, highest at the TSS and gradually declining toward the 3′ end of the genes (FIG. 3B), suggests that the PRC2-induced chromatin compaction follows the direction of the gene transcription. PRC2-bound promoters co-occupied by RNA polymerase II (RNAPII) exhibit significantly fewer interactions than PRC2-bound promoters lacking RNAPII binding (59% vs. 76%, p=0.0012, paired t-test) (FIG. 3C). Among all the genes bound by PRC2, those with PRC2-mediated chromatin interactions, including many well-known developmentally regulated genes like Wnt6-Ihh and Hoxb loci (FIG. 4A), have significantly lower steady-state levels of RNA (p=0.03). A similar pattern is also observed for genes with their promoter co-occupied with PRC2 and RNAPII (p-value <2.2e-16) (FIG. 3D). Many well-known developmentally regulated genes exhibited numerous PRC2-mediated loops of multiple interactions types (P-P, P-I, P-G and intra-G) as shown for the Hoxb loci. Among the 5,825 genes with BA-interactions, 3,784 (65%) display multiple 2) types of interactions (FIG. 3E) with the most common co-occurring types being P-P and intra-G looping (2871/3784; 76%), followed by P-P with either P-G or P-I (2648/3784; 70%), while only 7% of the genes display intra-G looping as the singular interaction type (FIG. 4B). Gene ontology analysis of these 5,825 genes suggests their functions significantly enriched in developmental processes (q-value=2e-140), morphogenesis (q-value=2.4e-96), and cellular differentiation (q-value=4.6e-86) (See Table 5), consistent with the known biological processes regulated by PRC2 in the pluripotent cells [Shen, X. et al. Mol Cell 32, 491-502 (2008)].

TABLE 5
Top 20 enriched Biological process in 5825 BA associated genes in relative to
24,692 non-interacting genes. Enrichment (N, B, n, b) is defined as: N—total number of
genes; B—total number of genes associated with a specific GO term; n—number of genes in
the top of the user's input list or in the target set when appropriate; b—number of genes in the
intersection; Enrichment = (b/n)/(B/N).

| GO term | Description | FDR q-value | Enrichment (N, B, n, b) |
|---|---|---|---|
| GO:0032502 | developmental process | 2.16E−140 | 1.65 (21172, 4680, 4642, 1692) |
| GO:0048856 | anatomical structure | 1.29E−133 | 1.80 (21172, 3273, 4642, 1292) |
| | development | | |
| GO:0048869 | cellular developmental | 2.47E−99 | 1.75 (21172, 2861, 4642, 1096) |
| | process | | |
| GO:0009653 | anatomical structure | 2.35E−96 | 2.09 (21172, 1463, 4642, 672) |
| | morphogenesis | | |
| GO:0051239 | regulation of multicellular | 1.96E−95 | 1.74 (21172, 2815, 4642, 1073) |
| | organismal process | | |
| GO:0048731 | system development | 6.42E−94 | 2.55 (21172, 771, 4642, 431) |
| GO:0007275 | multicellular organism | 9.33E−94 | 2.10 (21172, 1402, 4642, 647) |
| | development | | |
| GO:0050793 | regulation of | 1.55E−91 | 1.79 (21172, 2449, 4642, 959) |
| | developmental process | | |
| GO:0030154 | cell differentiation | 4.62E−86 | 1.83 (21172, 2108, 4642, 847) |
| GO:2000026 | regulation of multicellular | 1.11E−82 | 1.87 (21172, 1891, 4642, 775) |
| | organismal development | | |
| GO:0045595 | regulation of cell | 6.73E−78 | 1.90 (21172, 1687, 4642, 703) |
| | differentiation | | |
| GO:0048518 | positive regulation of | 1.77E−74 | 1.43 (21172, 5348, 4642, 1673) |
| | biological process | | |
| GO:0048522 | positive regulation of | 4.90E−73 | 1.46 (21172, 4789, 4642, 1529) |
| | cellular process | | |
| GO:0048513 | animal organ | 3.12E−66 | 1.97 (21172, 1281, 4642, 553) |
| | development | | |
| GO:0007267 | cell-cell signaling | 8.51E−60 | 2.61 (21172, 464, 4642, 266) |
| GO:0023052 | signaling | 3.20E−59 | 2.47 (21172, 547, 4642, 296) |
| GO:0051094 | positive regulation of | 4.53E−59 | 1.87 (21172, 1386, 4642, 569) |
| | developmental process | | |
| GO:0060284 | regulation of cell | 6.54E−58 | 2.08 (21172, 935, 4642, 426) |
| | development | | |
| GO:0051960 | regulation of nervous | 1.86E−57 | 2.09 (21172, 904, 4642, 415) |
| | system development | | |
| GO:0051240 | positive regulation of | 2.14E−57 | 1.78 (21172, 1633, 4642, 638) |
| | multicellular organismal | | |
| | process | | |

### Example 3

### PRC2-Interaction Anchors Act as Transcriptional Silencers

### (For Methods See Example 1)

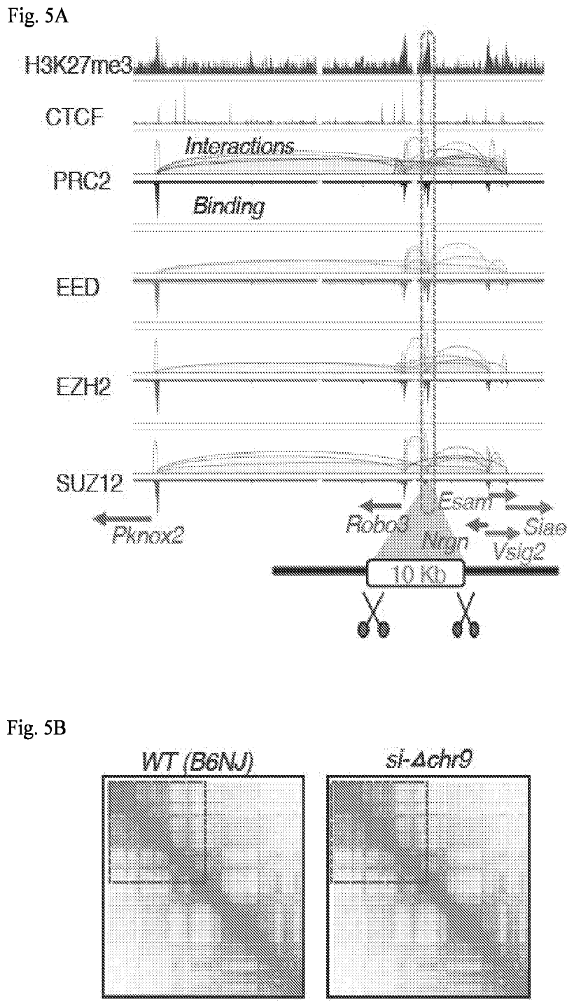

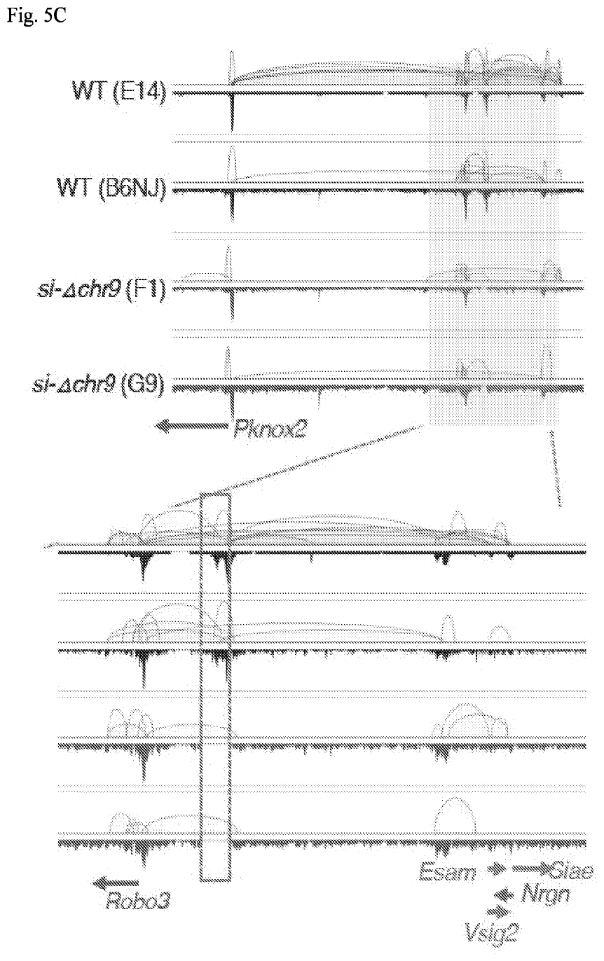

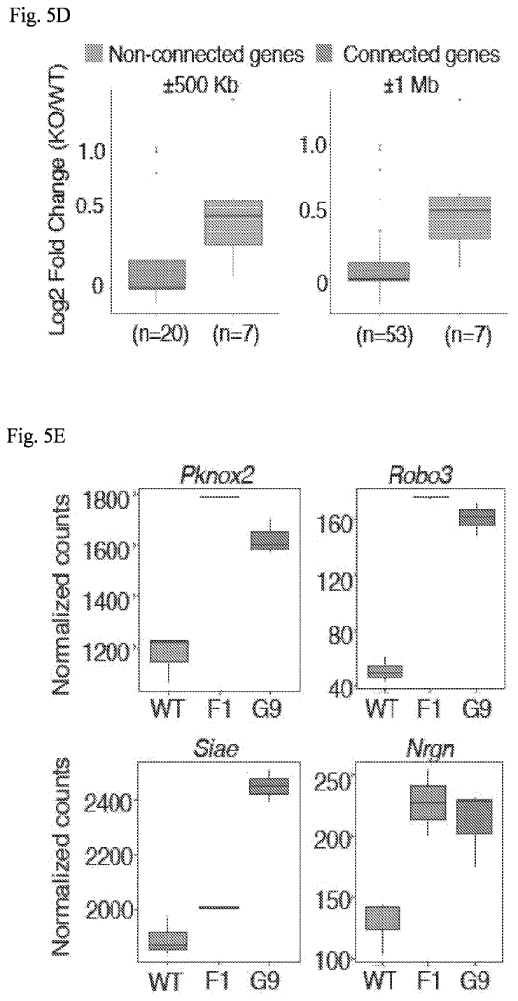

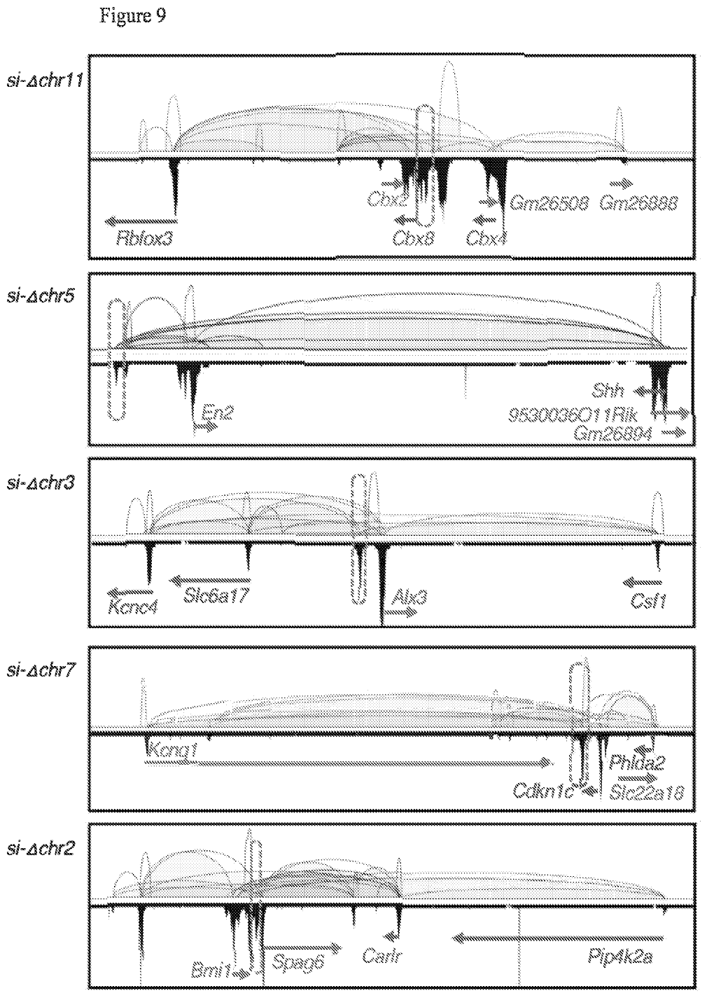

It was hypothesized that silencers can repress the transcription of their target genes through chromatin looping when bound by the repressors like PRC2, similar to how enhancers activate target gene expression [Brand, A. H., et al., Cell 41, 41-8 (1985) and Gray, S. & Levine, M. Curr Opin Cell Biol 8, 358-64 (1996)] (FIG. 4C). To test if the non-coding interacting anchors bound by PRC2 can function as the silencers, CRISPR/Cas9 targeted knockout (KO) of the intergenic anchors was performed, targeting deletion between 5-10 kb regions in sizes, and characterized gene expression changes in the KO mESCs (FIG. 6). From 21 intergenic anchors selected based on the functional importance of their connected genes (See Table 1), homozygous (−/−) KO mESC lines were successfully established for four loci. The deleted regions and their associated genes are shown in FIG. 5A and FIG. 9. Validation of the KO clones included Sanger sequencing runs and gels run which demonstrated validation of KO. Genotype was confirmed using Sanger sequencing of the PCR products for all six successfully generated KO clones. PCR genotyping of KO derived mES clones was also performed and confirmed deletion (deleted region on chromosome 9) in si-Δchr9 derived F1 and G9 clones. An additional primer R26 was designed to confirm heteroallelic deletion. The gender of the KO clones were determined to be XY and the wild type ES line was determined to be XX (see Methods in Examples). PCR genotyping was also performed and results confirmed deletion (deleted region on chromosome 7) in si-Δchr7 derived mES D4 and F4 clones. See Table 6 for partial sequences of the generated KO clones.

TABLE 6
Sequences from generated KO clones.

| | | SEQ | | SEQ ID |
|---|---|---|---|---|
| KO clone | Sequence | ID NO | Sequence | NO |
| Si-Δchr11 | ggaggggctcaaggtggctctcacagg | 123 | ccccagtccatgggattctactccttttgga | 124 |
| | tatagaccggggc. | | ggcagcaat | |
| Si-Δchr5 | cctccggtcccaccagtcatttgaacatg | 125 | tctacccgtgcgtgtcacaccgtacaggg | 126 |
| | gctaccatcca | | cgagatgtcat | |
| Si-Δchr9 | gaaatggagggtccaggaccctacac | 127 | aggggaagtaactaggttgtctgatgggtt | 128 |
| | agataggcttaatc | | cgttggcttc | |
| Si-Δchr3 | ccacaccccgccaaagctgctggccca | 129 | gacctgcaagcttatcatacaactgtggc | 130 |
| | caatctgggggag | | ctcccttccac | |
| Si-Δchr7 | ggaggtggctatctggtcaaccgtcttgg | 131 | agccagtgatgcataaatgcagtgcgcat | 132 |
| | cttcgagggaa | | gtgcaggaagc | |
| Si-Δchr2 | cgggggagcaccctgctcaaggcgcg | 133 | cactactagacccttaagacatacgaggt | 134 |
| | aacacccgggacg | | tgaccgtaaaa | |

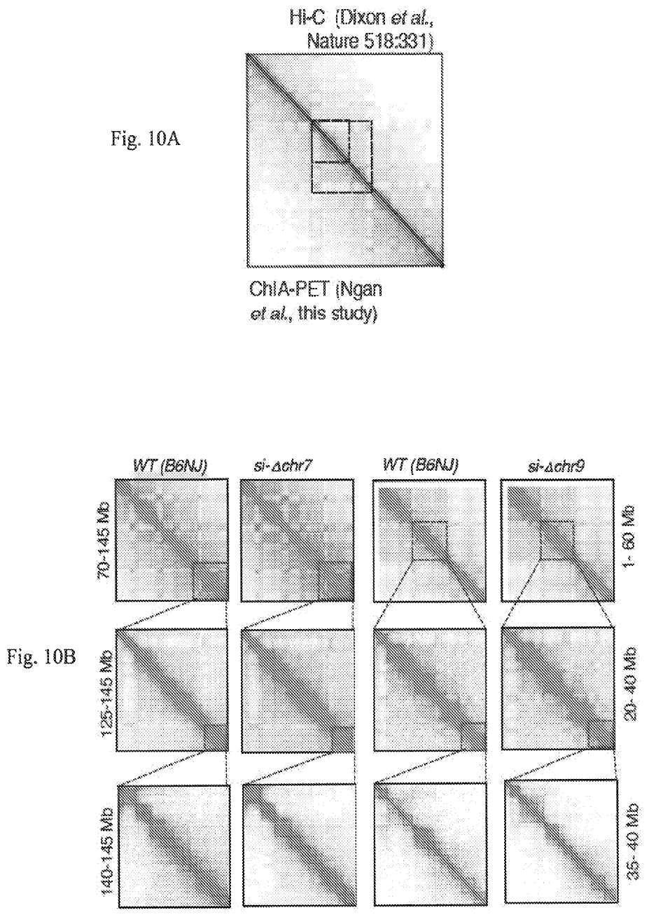

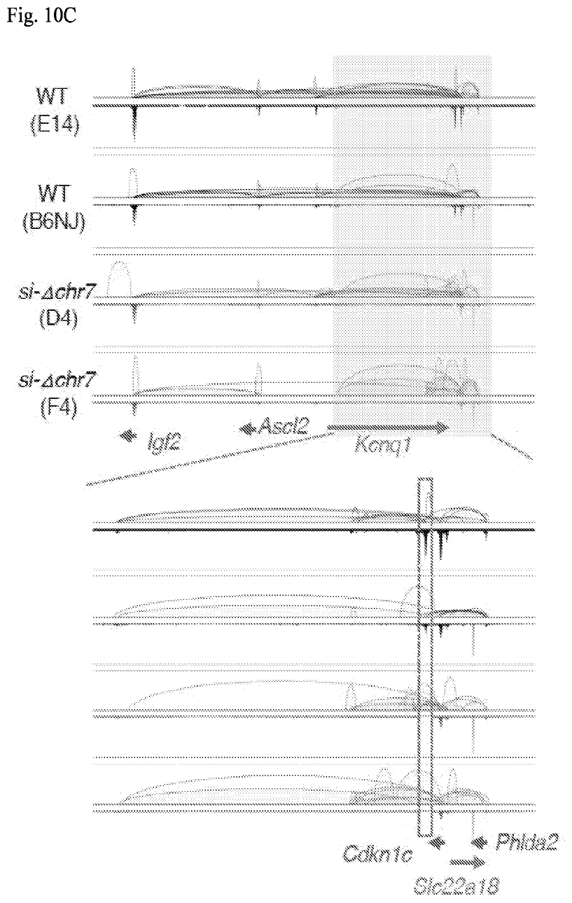

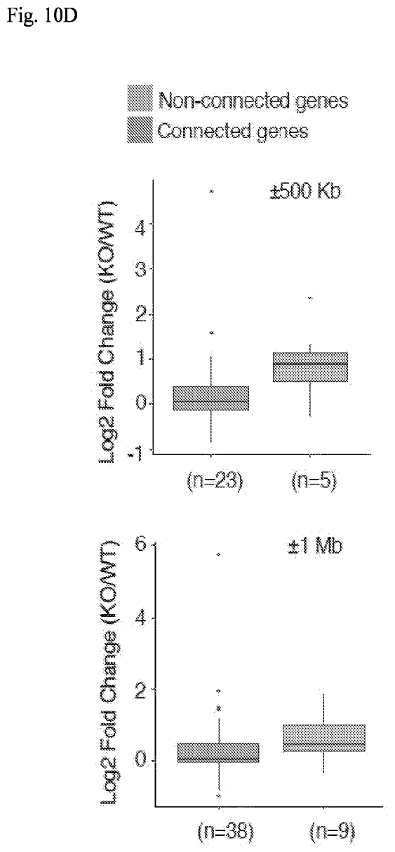

To evaluate the effects of interaction anchor deletion on chromatin organization, local interactions and PRC2 occupancy, particularly regions adjacent to the deleted loci and their targeted genes, the ChIA-PET analysis was performed in two of the KO ESC lines (si-Δchr9 and si-Δchr7) and the interaction maps were compared with those detected in the WT ES cells. ChIA-PET uncovered broad spatial topologically associating domains, similar to Hi-C-based approaches (FIG. 10A) and the topological structures surrounding the deleted regions did not yield any detectable changes while the local interactions originated from the deleted regions were lost (FIG. 5B-C and FIG. 10B-C). Also observed was an overall reduction of the interactions in the region proximity to si-Δchr9 locus when compared with WT ESCs. Despite the loss of specific interactions, PRC2 binding at these connected promoters was not affected.

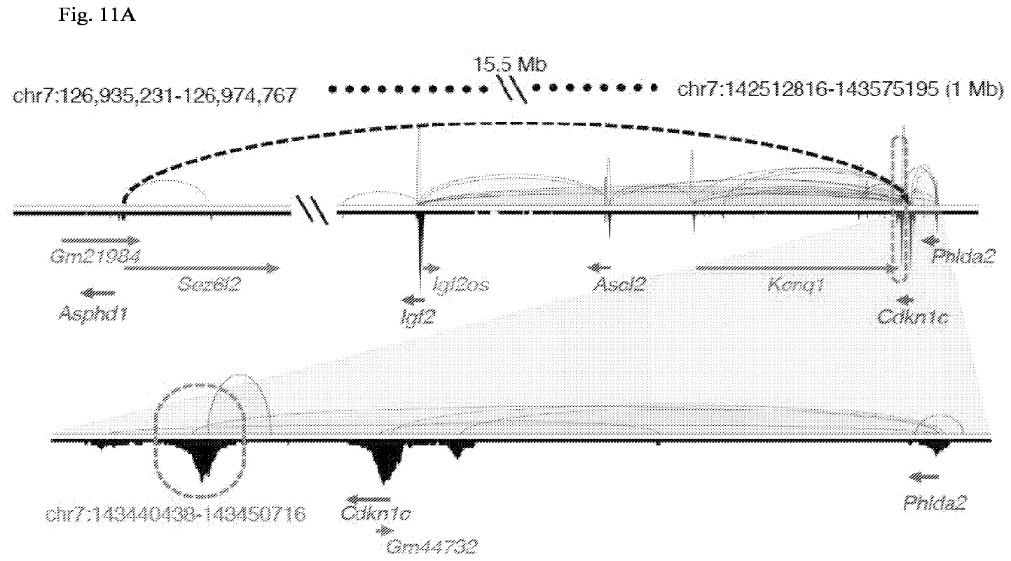

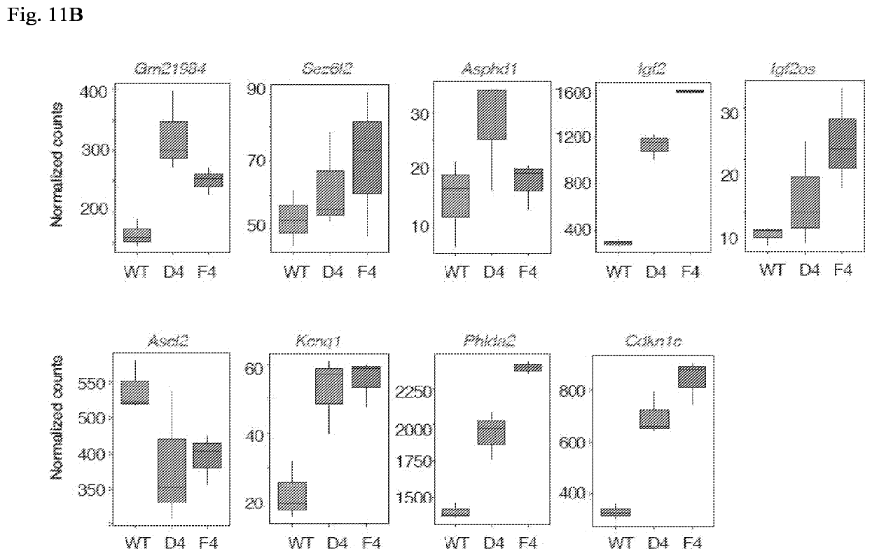

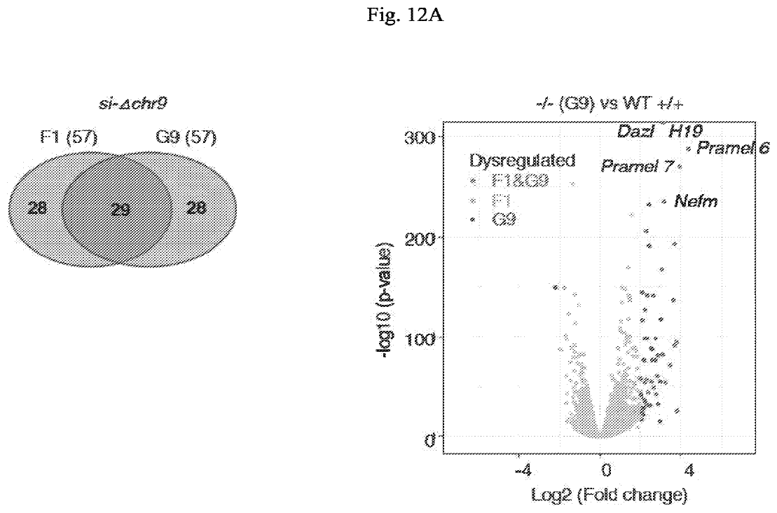

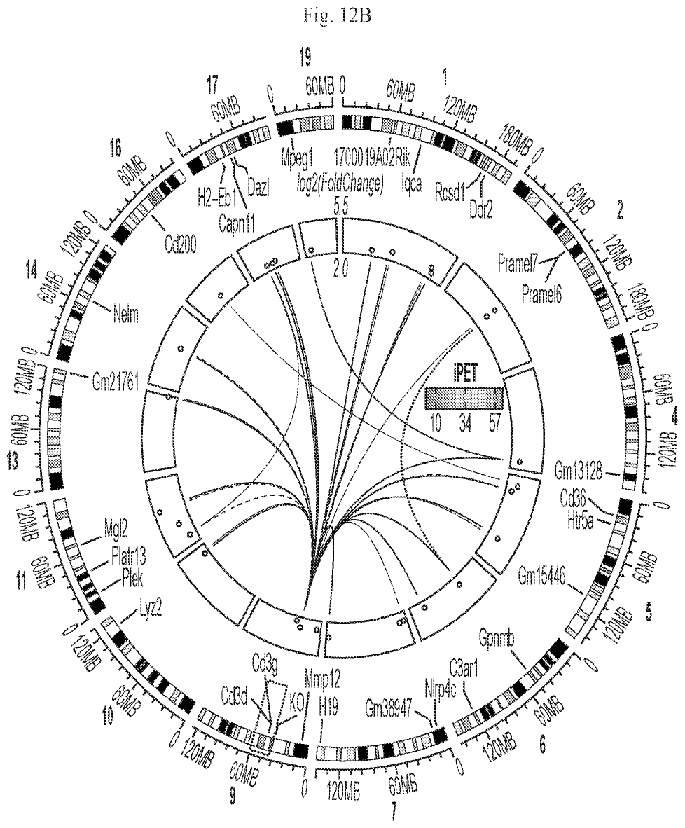

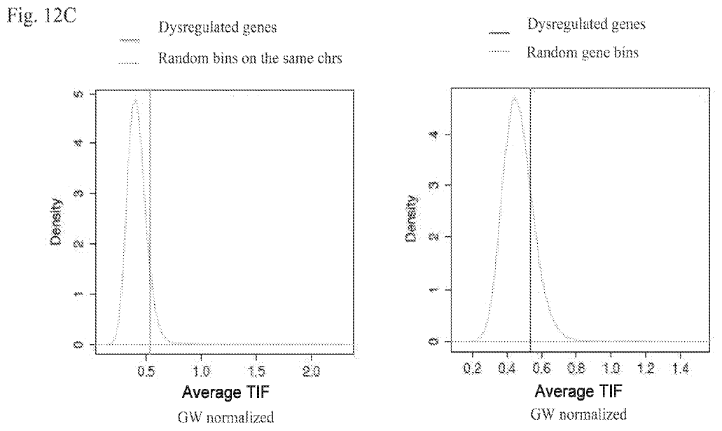

To determine the transcriptional effect associated with the deletion of the PRC2-bound silencers and their tethered interactions, the gene expression was compared between the homozygous deletion and wild type ESCs from multiple biological replicates. Genes interacting with the PRC2-bound interacting regions were overall upregulated (log 2 fold change >0) in the KO lines while the non-connected local genes (resided within ±500 Kb or ±1 Mb from the KO loci) were not (FIG. 5B, FIG. 10D). Specifically, in chr7, chr2 and chr3-silencer KOs (si-Δchr7, si-Δchr2 and si-Δchr3), the expression of 8 out of the 10, 9 out of 17 and 5 out of 9 connected genes were re-activated in KO ESC lines when compared with their expression in the wild-type ESCs, respectively (FIG. 11). In the chr9-silencer KO (si-Δchr9), excluding the 5 predicted genes whose expression cannot be detected, elevated RNA levels were detected from 7 of the 8 connected genes (FIG. 5E). The reactivation were observed from genes transcribed from both sides of the deleted anchors. Therefore, their silencing activities were independent of the direction of transcription. Beyond local re-activation, global transcriptional de-repression in si-Δchr9 ESCs was also observed. Among the differentially expressed genes (log 2 fold change >2 & P-adj <0.05) uncovered in si-Δchr9 ESCs, all except one, were upregulated in two independent F1 and G9 KO ESC lines (FIG. 5F, FIG. 12A). Genes exhibiting the most striking degree of de-repression were imprinting H19 lincRNA, tumor antigen Pramel6 and Dazl, a gene involved in spermatogenesis (See Table 7). It was reasoned that the transcriptional reactivation of these genes in the KO cells could be resulted from their spatial proximity to the si-chr9 locus in the 3-dimensional nuclear space and losing their contacts upon si-chr9 deletion. To evaluate their physical proximity, the trans-contacts between si-Δchr9 silencer locus and the derepressed genes were examined using the trans-interaction PETs in the ChIA-PET data and higher inter-chromosomal contact frequencies were observed between the dysregulated genes and the si-chr9 locus (FIG. 12B). To independently confirm their proximity in 3D space, studies further used the trans-chromosomal interaction frequencies (TIFs) determined in the mESC Hi-C data [Bonev, B. et al. Cell 171, 557-572 e24 (2017)]. The average TIFs between si-Δchr9 and the derepressed genes detected in the KO clones were determined and compared with the TIFs determined from the random background defined through either random loci selected from the same set of chromosomes where the dysregulated genes resided or genome-wide, non-dysregulated genes with 100,000 permutations. It was observed that the average TIF between si-chr9 silencer locus and the dysregulated genes was significantly higher (Wilcoxon tests' p-values <2.2E-16) using the genome-wide ICE (iterative correction and eigenvector decomposition)-normalized matrix [Imakaev, M. et al. Nat Methods 9, 999-1003 (2012)] (FIG. 12C). Given the increased expression of the genes coupled with the loss of their PRC2 associated promoter-silencer interactions, it was concluded that these PRC2-bound anchors function as transcriptional silencers.

TABLE 7
Gene derepression results from certain experiments.

| | clone F1 | | clone G9 | | | |
|---|---|---|---|---|---|---|
| Gene | log2fold | adjusted | log2fold | adjusted | | Proposed |
| name | change | p-value | change | p-value | Gene ID | function |
| 170001 | 2.281553573 | 6.61E−34 | 3.696246467 | 1.51E−88 | protein_ | |
| 9A02Rik | | | | | coding | |
| C3ar1 | 2.325252249 | 2.38E−39 | 2.558583473 | 8.98E−53 | protein_ | complement |
| | | | | | coding | component 3a |
| | | | | | | receptor 1, |
| | | | | | | promote both |
| | | | | | | basal and |
| | | | | | | ischemia-induced |
| | | | | | | neurogenesis |
| Capn11 | 2.216764438 | 1.13E−102 | 2.138405728 | 5.23E−114 | protein_ | Calcium-activated |
| | | | | | coding | neutral |
| | | | | | | proteinase 11, |
| | | | | | | Expressed |
| | | | | | | exclusively in |
| | | | | | | testis |
| Cd200 | 2.946214999 | 1.78E−42 | 2.784811496 | 3.78E−41 | protein_ | plays a central |
| | | | | | coding | role in |
| | | | | | | macrophage |
| | | | | | | fusion and |
| | | | | | | osteoclast |
| | | | | | | formation |
| Cd36 | 2.9649528 | 2.35E−85 | 2.602919469 | 3.02E−74 | protein_ | Multifunctional |
| | | | | | coding | glycoprotein that |
| | | | | | | acts as receptor |
| | | | | | | for a broad range |
| | | | | | | of ligands |
| Cd3d | 3.252751916 | 2.90E−66 | 3.783961921 | 2.58E−92 | protein_ | T-CELL |
| | | | | | coding | ANTIGEN |
| | | | | | | RECEPTOR |
| | | | | | | COMPLEX, |
| | | | | | | mediates signal |
| | | | | | | transduction |
| Cd3g | 3.397570639 | 3.34E−63 | 3.459383036 | 6.80E−69 | protein_ | T-CELL |
| | | | | | coding | ANTIGEN |
| | | | | | | RECEPTOR |
| | | | | | | COMPLEX, |
| | | | | | | mediates signal |
| | | | | | | transduction |
| DazI | 2.375817442 | 0 | 3.088839536 | 0 | protein_ | spermatogenesis, |
| | | | | | coding | initiate meiosis in |
| | | | | | | response to |
| | | | | | | retinoic acid |
| Ddr2 | 2.587225653 | 2.42E−97 | 2.359328388 | 4.05E−96 | protein_ | receptor protein |
| | | | | | coding | tyrosine kinase |
| | | | | | | (RTK) |
| Gm13128 | 2.584927875 | 2.10E−34 | 2.305280485 | 1.39E−30 | protein_ | |
| | | | | | coding | |
| Gm15446 | 2.974328714 | 3.82E−135 | 3.05368027 | 4.64E−165 | lincRNA | |
| Gm21761 | 2.907711389 | 3.65E−102 | 2.013782045 | 1.11E−55 | protein_ | |
| | | | | | coding | |
| Gm38947 | 2.466786906 | 2.50E−15 | 3.819167637 | 1.41E−24 | lincRNA | |
| Gpnmb | 2.719217797 | 1.45E−189 | 2.472351242 | 1.57E−188 | protein_ | Transmembrane |
| | | | | | coding | glycoprotein, |
| | | | | | | Could be a |
| | | | | | | melanogenic |
| | | | | | | enzyme |
| H19 | 3.598108666 | 0 | 3.128991068 | 0 | lincRNA | imprinted |
| | | | | | | maternally |
| | | | | | | expressed |
| | | | | | | transcript, a |
| | | | | | | developmentally |
| | | | | | | regulated gene |
| | | | | | | with putative |
| | | | | | | tumor suppressor |
| | | | | | | activity |
| H2-Eb1 | 2.210094937 | 4.12E−102 | 2.370884401 | 5.55E−139 | protein_ | histocompatibility |
| | | | | | coding | 2, class II antigen |
| | | | | | | E beta |
| Htr5a | 2.304618236 | 2.83E−34 | 2.407481173 | 3.50E−42 | protein_ | Serotonin |
| | | | | | coding | receptor 5A |
| Iqca | 2.61897153 | 3.17E−64 | 3.639807313 | 1.24E−134 | protein_ | IQ motif |
| | | | | | coding | containing with |
| | | | | | | AAA domain |
| Lyz2 | 2.203479922 | 2.30E−152 | 2.308409158 | 2.41E−203 | protein_ | Lysozymes |
| | | | | | coding | |
| Mgl2 | 2.179740885 | 2.04E−25 | 3.212728771 | 9.40E−52 | protein_ | macrophage |
| | | | | | coding | galactose N- |
| | | | | | | acetyl- |
| | | | | | | galactosamine |
| | | | | | | specific lectin 2 |
| Mmp12 | 2.742575289 | 2.56E-34 | 2.102254365 | 3.17E−24 | protein_ | matrix |
| | | | | | coding | metallopeptidase |
| | | | | | | 12, May be |
| | | | | | | involved in tissue |
| | | | | | | injury and |
| | | | | | | remodeling |
| Mpeg1 | 2.129075611 | 6.65E−41 | 2.223885002 | 8.27E−52 | protein_ | macrophage |
| | | | | | coding | expressed gene 1 |
| Nefm | 3.334944451 | 9.05E−226 | 3.175011114 | 7.90E−233 | protein_ | an intermediate- |
| | | | | | coding | sized cytoskeletal |
| | | | | | | neurofilament |
| | | | | | | that localizes to |
| | | | | | | neuronal axons |
| | | | | | | and dendrites |
| Nlrp4c | 2.783692971 | 1.40E−95 | 3.71114131 | 5.83E−190 | protein_ | May be involved |
| | | | | | coding | in inflammation |
| Platr13 | 3.305886456 | 8.24E−97 | 2.336061956 | 3.37E−55 | lincRNA | |
| Plek | 2.523194075 | 1.25E−38 | 2.664333714 | 3.29E−47 | protein_ | Major protein |
| | | | | | coding | kinase C |
| | | | | | | substrate of |
| | | | | | | platelets |
| Pramel6 | 4.586419884 | 6.59E−286 | 4.402792745 | 3.58E−284 | protein_ | preferentially |
| | | | | | coding | expressed |
| | | | | | | antigen in |
| | | | | | | melanoma like 6 |
| Pramel7 | 4.15461037 | 8.64E−264 | 3.985107654 | 3.14E−267 | protein_ | preferentially |
| | | | | | coding | expressed |
| | | | | | | antigen in |
| | | | | | | melanoma like 7 |
| Rcsd1 | 2.724573694 | 7.26E−82 | 2.75999872 | 7.96E−96 | protein_ | actin filament |
| | | | | | coding | binding |

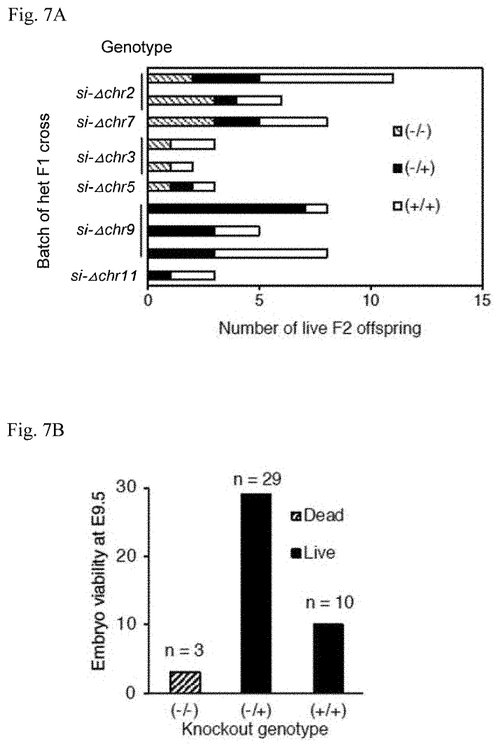

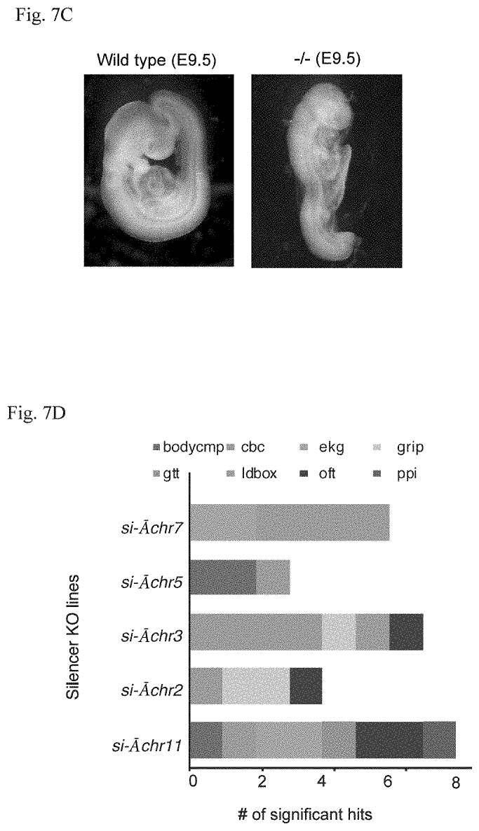

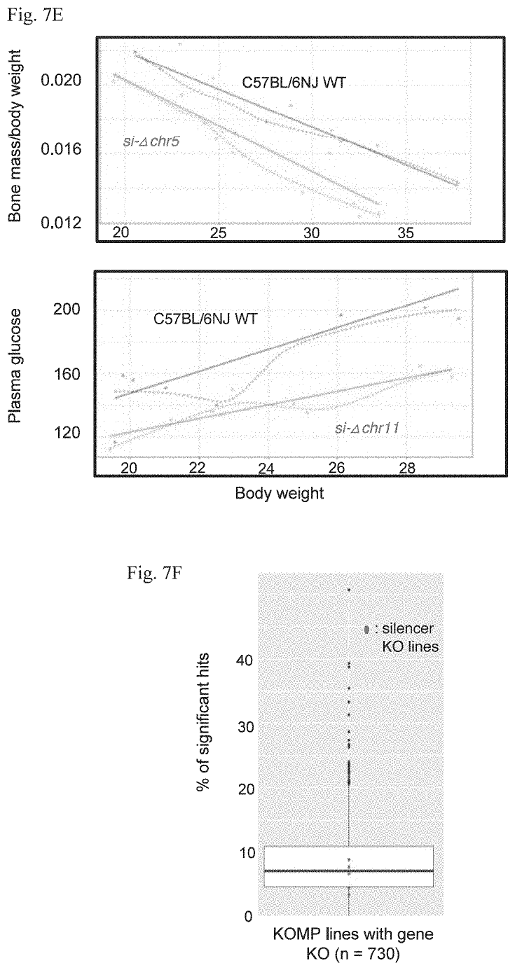

To characterize of the in vivo function of the PRC2-bound silencers, the phenotypes associated with the homozygous silencer deletions in mice were assessed. From total six PRC2-bound silencer KOs for which heterozygous mice were successfully established, viable homozygous KO mice were derived from five of them (FIG. 7A). Heterozygous mice carrying si-Δchr9 failed to yield any viable homozygous (−/−) pups from three separate crosses, indicating an essential function of si-chr9 in the embryonic development. To reaffirm the embryonic lethality, embryos were examined at embryonic day 9.5 (E9.5), the earliest stage when they can be recognized, and no viable homozygous (−/−) embryos were found (FIG. 7B). Among the three dead embryos carrying homozygous deletions, one of which showed morphology of developmentally delay (FIG. 7C), and the other two were resorbed. From the remaining five PRC2-bound silencer loci with viable homozygous KO mice, a comprehensive, standardized phenotyping screen was conducted that measured 126 phenotyping parameters in 14 test procedures encompassing diverse biological and disease areas [Dickinson, M. E. et al. Nature 537, 508-514 (2016) and Meehan, T. F. et al. Nat Genet 49, 1231-1238 (2017)]. From these five silencer KO lines, significant variation (FDR <0.05) was detected in 28 phenotypic measurements, ranging between three to eight different assays per KO line, from eight different procedures (Table 8; FIG. 7D), including lower bone mass, plasma glucose level (FIG. 7E) and grip strength. In si-Δchr3 KO mice, three of the seven significant phenotypical aberrations were involved in the blood cell counts (cbc) while in si-Δchr7 KO mice, four of the six significant changes were anxiety responses measured by light-dark box tests (ldbox). Intriguingly, in si-Δchr11 KO mice, three of the eight significant phenotypical aberrations, namely heart rate, R-R interval in electrocardiography (ekg) and startle responses, measured by the prepulse inhibition (ppi) tests, were also altered in the mutant strains of CBX4 and Rbfox3, the two genes connecting to this particular silencer locus through PRC2 bound chromatin loops, suggesting a possible mechanistic model for these DREs' function. Overall, the ratio of the significant hits detected from these noncoding silencer KOs were comparable to those from the coding-gene KO strains (n=730 strains) (FIG. 7F), suggesting that these PRC2-bound DREs were of equivalent functional importance to the protein-coding genes. Collectively, the pleiotropic phenotypic aberration observed in six silencer KO strains provided functional annotation of these PRC2-bound DREs in vivo and highlighted the importance of their biological roles during development.

TABLE 8
Results of eight different procedures on five silencer KO lines.

| Phenotype | | Domain | Ctrl.F.N | Ctrl.M.N | Mt.F.N |
|---|---|---|---|---|---|
| si-Δchr11 | bmc_over_bw | bodycmp | 17 | 17 | 8 |
| | corrected_percent_pp2_vmax | ppi | 9 | 10 | 8 |
| | distance_traveled_slope | oft | 9 | 10 | 8 |
| | hr | ekg | 9 | 10 | 8 |
| | number_of_rears_total | oft | 9 | 10 | 8 |
| | plasma_glucose_120_min | gtt | 8 | 10 | 5 |
| | rr | ekg | 9 | 10 | 8 |
| | white_blood_cells_wbc | cbc | 6 | 7 | 6 |
| si-Δchr2 | center_average_speed | oft | 10 | 10 | 8 |
| | fore_and_hind_limb_grip_mean | grip | 10 | 10 | 8 |
| | fore_and_hind_limb_grip_meanbody_weight | grip | 10 | 10 | 8 |
| | white_blood_cells_wbc | cbc | 11 | 11 | 6 |
| si-Δchr3 | fore_and_hind_limb_grip_mean | grip | 10 | 10 | 8 |
| | mean_platelet_volume_mpv | cbc | 7 | 7 | 5 |
| | neutrophils_neut | cbc | 7 | 7 | 5 |
| | pct_time_in_dark | ldbox | 9 | 10 | 7 |
| | pcttime_corners_slope | oft | 10 | 10 | 8 |
| | red_cell_hem_con_mean_chcm | cbc | 7 | 7 | 5 |
| | white_blood_cells_wbc | cbc | 7 | 7 | 5 |
| si-Δchr5 | bmc_over_bw | bodycmp | 5 | 5 | 7 |
| | bone_area | bodycmp | 5 | 5 | 7 |
| | right_side_mobile_time_spent | Id box | 10 | | |
| si-Achr7 | hr | ekg | 15 | 13 | 8 |
| | left_side_mobile_time_spent | Id box | 5 | 5 | 8 |
| | pct_time_in_dark | Id box | 5 | 5 | 8 |
| | right_side_mobile_time_spent | Id box | 5 | 5 | 8 |
| | rr | ekg | 15 | 13 | 8 |
| | side_changes | Id box | 5 | 5 | 8 |

| Genotype | Mt.M.N | genotype_estimate | genotype_estimate_SE | genotype_p_value |
|---|---|---|---|---|
| si-Δchr11 | 8 | −0.001128945 | 0.000339138 | 0.015828883 |
| | 8 | 0.231692166 | 0.071607205 | 0.002954166 |
| | 8 | −22.06182628 | 7.144488997 | 0.004226459 |
| | 8 | 34.33584332 | 10.17519574 | 0.002057098 |
| | 8 | 42.26867958 | 9.249997718 | 7.35E−05 |
| | 4 | −33.71823562 | 9.508565339 | 0.001811997 |
| | 8 | −4.091566262 | 1.201050086 | 0.001890186 |
| | 6 | 2.34425926 | 0.738755484 | 0.004578783 |
| si-Δchr2 | 6 | 1.569932351 | 0.419727662 | 0.000839142 |
| | 8 | −26.50865941 | 5.983285476 | 0.000109041 |
| | 8 | −1.308548081 | 0.290549084 | 9.42E−05 |
| | 4 | −2.703009696 | 1.095117243 | 0.020473734 |
| si-Δchr3 | 8 | −15.74687138 | 6.271782291 | 0.017475015 |
| | 6 | 0.550350772 | 0.162738594 | 0.002815717 |
| | 6 | −1.775470317 | 0.481173363 | 0.001360421 |
| | 7 | −9.126204102 | 3.150750552 | 0.00710702 |
| | 6 | −7.006576271 | 1.829132347 | 0.000606995 |
| | 6 | −0.772157858 | 0.173165808 | 0.000240669 |
| | 6 | 4.006571777 | 0.766822921 | 3.53E−05 |
| si-Δchr5 | 7 | −0.001611774 | 0.000555708 | 0.009537194 |
| | 7 | −0.741716675 | 0.221367015 | 0.003359144 |
| | 10 | −42.09307672 | 14.7626656 | 0.007453201 |
| si-Δchr7 | 6 | 21.11110838 | 7.561917432 | 0.008245565 |
| | 8 | 89.67731609 | 13.94183995 | 2.25E-06 |
| | 8 | −13.71477836 | 4.612386754 | 0.007247781 |
| | 8 | 67.37489987 | 24.57569949 | 0.012578599 |
| | 6 | −2.246512195 | 0.909434524 | 0.018236344 |
| | 8 | 59.87676239 | 10.79588332 | 1.67E−05 |

| Phenotype | sex_estimate | sex_estimate_SE | sex_p_value | weight_estimate | weight_estimate_SE | weight_p_value |
|---|---|---|---|---|---|---|
| si-Δchr11 | −0.0041569 | 0.000651097 | 1.36E−07 | −9.29E−05 | 7.54E−05 | 0.224987137 |
| | −0.064856537 | 0.071082243 | 0.368824416 | NA | NA | NA |
| | 9.869871428 | 7.076509872 | 0.17300976 | NA | NA | NA |
| | 25.01204824 | 10.14188895 | 0.019591092 | NA | NA | NA |
| | −4.644404609 | 9.177926118 | 0.616407958 | NA | NA | NA |
| | 3.634093596 | 15.24015927 | 0.813735236 | 5.815913785 | 2.196067427 | 0.014680922 |
| | −2.979518076 | 1.197118651 | 0.018588359 | NA | NA | NA |
| | 4.159074074 | 0.738755484 | 1.38E−05 | NA | NA | NA |
| si-Δchr2 | −2.190243348 | 0.802486125 | 0.010845052 | 0.228358393 | 0.13999488 | 0.114048634 |
| | 17.34915904 | 5.988285341 | 0.006850642 | NA | NA | NA |
| | 1.293008689 | 0.660424993 | 0.05960645 | −0.476645966 | 0.103854632 | 7.40E−05 |
| | 5.251835293 | 1.918166761 | 0.011007701 | 0.079465294 | 0.201182705 | 0.696071676 |
| si-Δchr3 | −13.21352301 | 13.42996833 | 0.332789964 | 4.776867191 | 2.046186375 | 0.026221486 |
| | 0.107765505 | 0.160187843 | 0.508445652 | NA | NA | NA |
| | −0.08798462 | 0.475925151 | 0.855104215 | NA | NA | NA |
| | −3.634285699 | 3.11572674 | 0.252942591 | NA | NA | NA |
| | 1.550932204 | 1.803548817 | 0.396645797 | NA | NA | NA |
| | 0.354573794 | 0.361606795 | 0.338527765 | −0.042257667 | 0.03538514 | 0.2463636 |
| | 4.942315661 | 0.758471051 | 1.87E−06 | NA | NA | NA |
| si-Δchr5 | −0.000372977 | 0.000886481 | 0.678926286 | −0.000397638 | 9.48E−05 | 0.000544319 |
| | 0.424181132 | 0.188183634 | 0.036191722 | NA | NA | NA |
| | −24.36269874 | 13.65695724 | 0.083638056 | NA | NA | NA |
| si-Δchr7 | 16.40530651 | 7.358063353 | 0.031929124 | NA | NA | NA |
| | −96.73656738 | 11.08275755 | 1.98E−08 | NA | NA | NA |
| | 18.64604379 | 3.694985372 | 5.37E−05 | NA | NA | NA |
| | −107.2136785 | 35.91862239 | 0.007320307 | 6.660614465 | 5.502550107 | 0.240214228 |
| | −1.723020406 | 0.883421498 | 0.058735364 | NA | NA | NA |
| | −61.09229174 | 8.596171993 | 5.20E−07 | NA | NA | NA |

### Example 4

PRC2-Associated Silencers Transition into Active Enhancers During Differentiation

### (For Methods See Example 1)

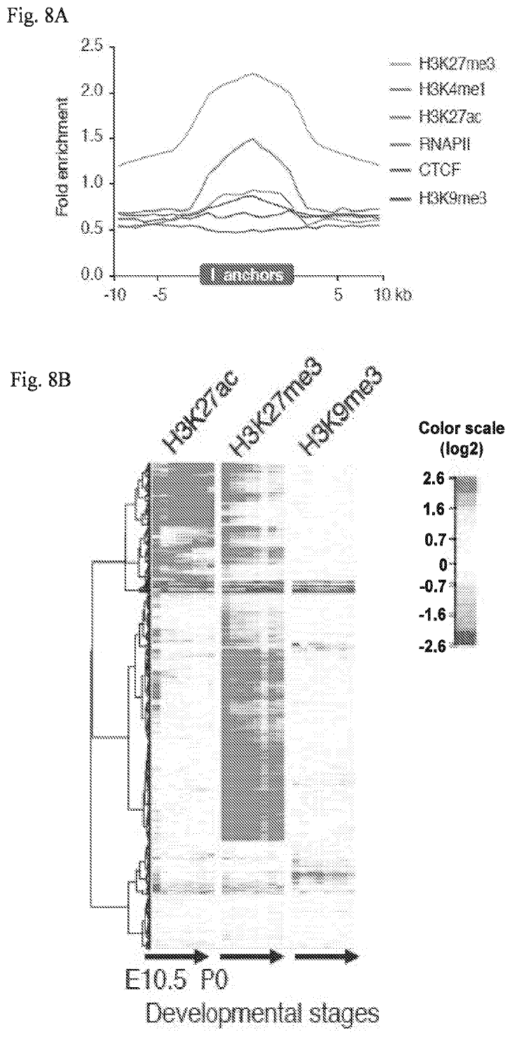

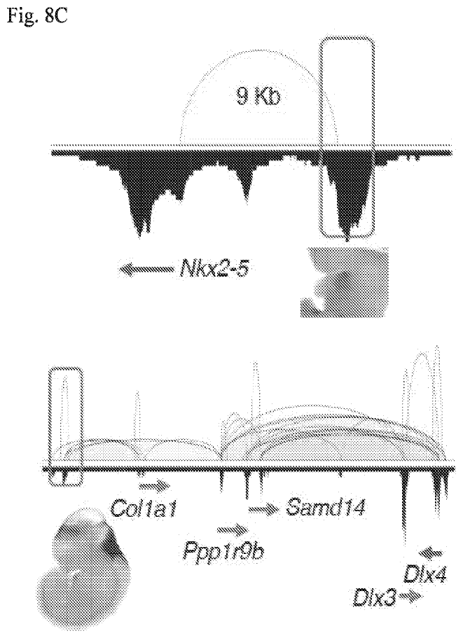

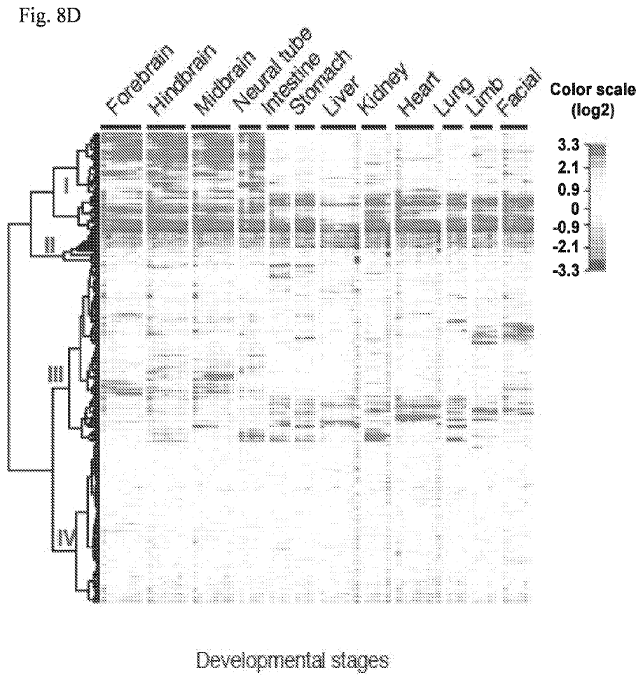

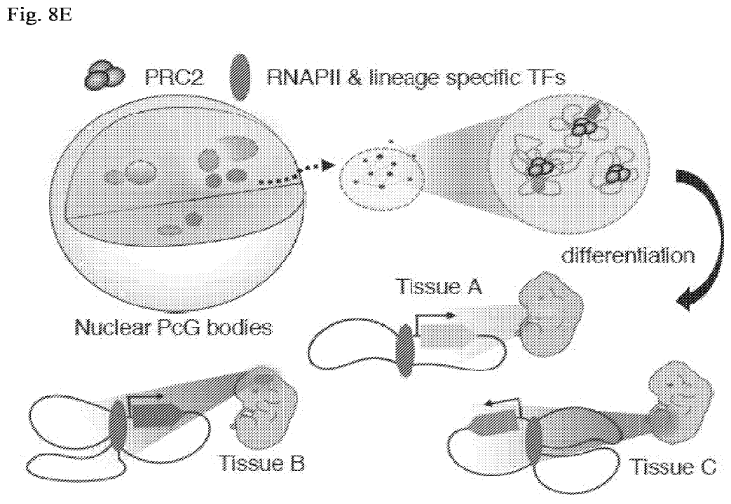

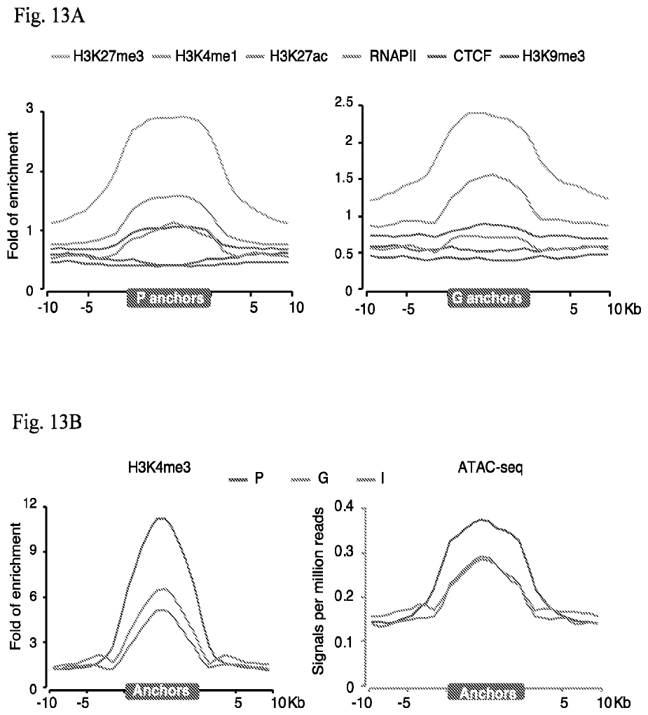

To characterize the chromatin states and functional features of the PRC2-bound silencers, profiles of chromatin signatures representing open chromatin accessibility (ATAC-seq), active and repressive histone modifications (H3K4me3, H3K4me1, H3K27ac, H3K27me3, H3K9me3) as well as RNAPII and insulator CTCF binding were either downloaded from ENCODE or generated in this study (see Example 1 Methods). Their enrichment within the non-coding intergenic interaction anchors (I, n=1,800), the promoter anchors (P, n=4,120) and intragenic anchors (G, n=2,302) were examined. For all three anchor types, the enrichment of H3K4me3 and ATAC-seq signals was observed, as well as a moderate co-enrichment of H3K27me3 and H3K4me1, a signature previously identified for poised enhancers [Rada-Iglesias, A. et al. Nature 470, 279-83 (2011)] (FIG. 8A and FIG. 13). These PRC2-bound silencers were also surveyed for the enrichment of regulatory function, including open chromatin and transcription factor binding (TFBS) annotated in the ENSEMBL regulatory build [Zerbino, D. R., et al., Genome Biol 16, 56 (2015)], the key pluripotent TFBS [Chen, X. et al. Cell 133, 1106-17 (2008)], and the CpG islands (CGI) from UCSC Genome Browser [Gardiner-Garden, M. & Frommer, M. J Mol Biol 196, 261-82 (1987)]. CGI was found with highest enrichment (average log 2 fold enrichment 6.8), consistent to its involvement in PcG recruitment [Deaton, A. M. & Bird, A. Genes Dev 25, 1010-22 (2011) and Ku, M. et al. PLoS Genet 4, e1000242 (2008)]. Regulatory features like open chromatin and TF binding were found over represented but enhancer signal was depleted in the PRC2-bound silencers (See Table 9). Furthermore, most of the TFs important for self-renewal and pluripotency, like MYC, SMAD1, were also found enriched in these PRC2-bound silencers, raising the possibility that these PRC2-bound silencers are the foci for multiple TF binding and these TFs could be important for PRC2-mediated transcriptional repression.

TABLE 9
Regulatory feature information.

| | | | Z score |
|---|---|---|---|
| | | log2 Fold | (1000X |
| | Features | Enrichment | simulation) |
| Ensembl Regulatory | Open chromatin | 1.05 | 11.58 |
| Build | TF binding | 1.06 | 5.68 |
| (PMID: 25887522) | Enhancer | −1.76 | -6.86 |
| TF binding | n-Myc | 4.4 | 26.05 |
| (PMID: 18555785) | Smad1 | 4 | 14.68 |
| | ZFX | 3.8 | 26.32 |
| | Pou5f1 | 3.6 | 17.9 |
| | Klf4 | 3.5 | 25.7 |
| | c-Myc | 3.5 | 8.54 |
| | Esrrb | 3.4 | 37.51 |
| | Tcfcp2l1 | 3.2 | 33.65 |
| | STAT3 | 3.1 | 10.27 |
| | Sox2 | 3 | 14.23 |
| | E2F1 | 2.8 | 17.1 |
| | CTCF | 2.6 | 28.21 |
| | Nanog | 2.5 | 14.74 |
| Sequence | CGI | 6.8 | 176.37 |
| composition | | | |

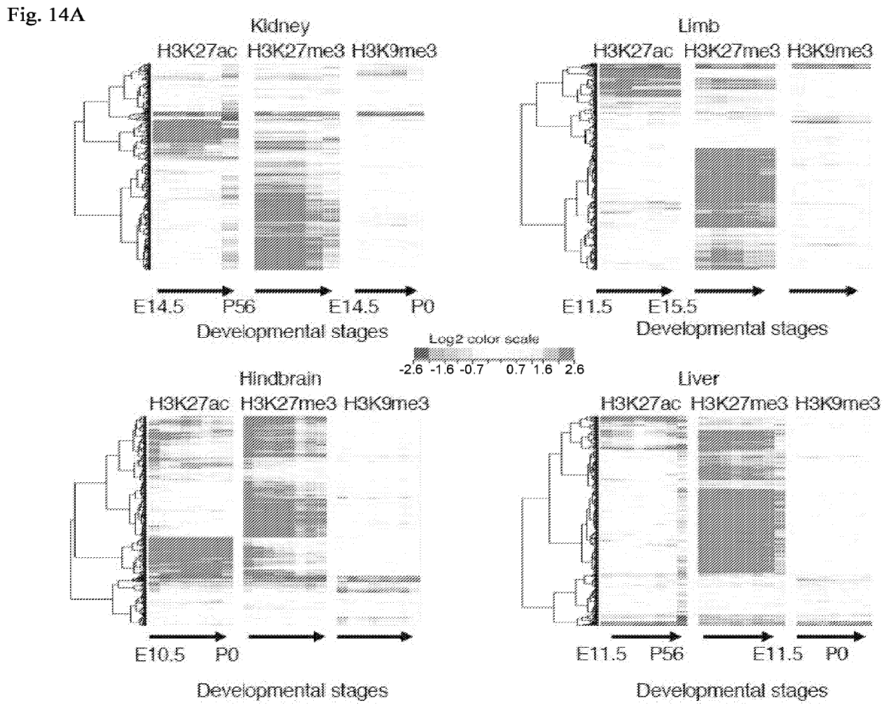

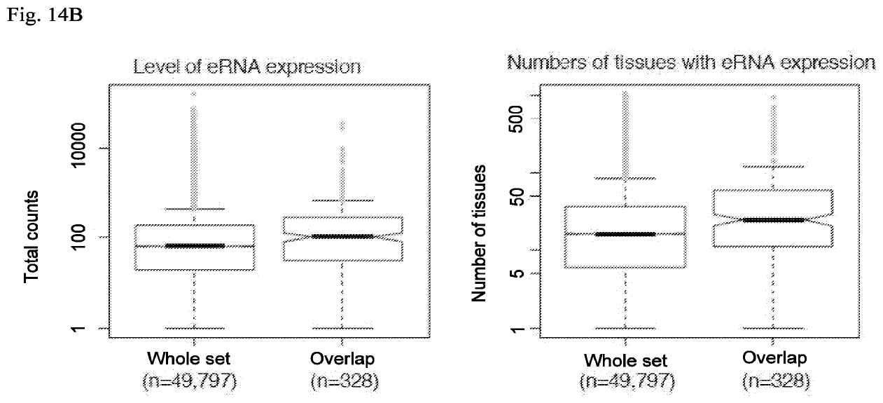

The enrichment of poised enhancer signature within the intergenic anchors suggests that these PRC2-bound silencers could transition into enhancers during ESC differentiation. To test this hypothesis, the histone modification representing active enhancers (H3K27ac) were surveyed and two repressive marks (H3K27me3 and H3K9me3) in these silencers throughout embryonic differentiation stages from E10.5 to postnatal day 0 (P0) or day 56 (P56) across 12 major mouse tissues (See Table 10) listed all ENCODE data used). In each tissue type, H3K27ac enrichment was detected with partial reduction of H3K27me3 in a subset of these regions (FIG. 8B, FIG. 14A). To further verify their enhancer activities in the differentiated tissues, searches for the presence of validated mouse enhancers within these regions were performed from the collection of the VISTA enhancer identified in mice (//enhancer.lbl.gov) [Visel, A., et al., Nucleic Acids Res 35, D88-92 (2007)] and eRNA expression in the developed tissues profiled by CAGE analysis in FANTOMS [Arner, E. et al. Science 347, 1010-4 (2015)]. 28 of the PRC2 bound DREs were found displaying VISTA enhancer activities in tissues ranging from heart, hindbrain to limb (FIG. 8C). Furthermore, 25% of PRC2-bound DREs exhibited eRNA expression. Compared with all FANTOMS-defined enhancer regions (n=49,797), the regions overlapped with PRC2 bound DREs (n=328) expressed eRNAs in more cell types (mean 70 vs 36; p-value 3.84E-11, one-sided Wilcoxon test) and at higher level (mean normalized CAGE tag counts 589 vs 274; p-value 3.84E-11) (FIG. 14B). It was possible to stratify the PRC2-bound silencers into four separate groups (FIG. 8D, Table 11) based on their overall H3K27ac enrichment pattern across 74 developmental stages from 12 major tissue types. Group I DREs (n=371) exhibit H3K27ac signal in multiple tissues and developmental stages, thus potentially functioning as common enhancers in multiple lineages. Group II DREs (n=126) are strongly devoid of H3K27ac signal across all cell types and stages, possibly inert or inaccessible regions. Group III DREs (n=683) exhibits enriched H3K27ac in only a few selective tissues or stages, suggesting their stage-specific enhancer activities; and Group IV DREs (n=620) show little H3K27ac enrichment in any cell type surveyed here; these could be enhancers in other tissue types not included here.

TABLE 10
Results of survey of histone modification representing active enhancers (H3K27ac)
and two repressive marks (H3K27me3 and H3K9me3) in these silencers throughembryonic
differentiation stages from E10.5 to postnatal day 0 (PO) or day 56 (P56) across 12 major
mouse tissues. All ENCODE data used

| Histone | | Age | | |
|---|---|---|---|---|
| modifications | Cell | (days) | Encode_fileID | Download_link |
| H3K27ac | forebrain | 10.5 | ENCFF266KHC | www.encodeproject.org/files/ENCFF266KHC/@ |
| | | | | @download/ENCFF266KHC.bigWig |
| H3K27ac | forebrain | 11.5 | ENCFF827WXG | www.encodeproject.org/files/ENCFF827WXG/@ |
| | | | | @download/ENCFF827WXG.bigWig |
| H3K27ac | forebrain | 12.5 | ENCFF541IJI | www.encodeproject.org/files/ENCFF541IJI/@@ |
| | | | | download/ENCFF541IJI.bigWig |
| H3K27ac | forebrain | 13.5 | ENCFF107UHG | www.encodeproject.org/files/ENCFF107UHG/@ |
| | | | | @download/ENCFF107UHG.bigWig |
| H3K27ac | forebrain | 14.5 | ENCFF527FOD | www.encodeproject.org/files/ENCFF527FOD/@ |
| | | | | @download/ENCFF527FOD.bigWig |
| H3K27ac | forebrain | 15.5 | ENCFF440SCC | www.encodeproject.org/files/ENCFF440SCC/@ |
| | | | | @download/ENCFF440SCC.bigWig |
| H3K27ac | forebrain | 16.5 | ENCFF826KEG | www.encodeproject.org/files/ENCFF826KEG/@ |
| | | | | @download/ENCFF826KEG.bigWig |
| H3K27ac | forebrain | 20 | ENCFF835DLZ | www.encodeproject.org/files/ENCFF835DLZ/@ |
| | | | | @download/ENCFF835DLZ.bigWig |
| H3K27me3 | forebrain | 10.5 | ENCFF063ZLI | www.encodeproject.org/files/ENCFF063ZLI/@@ |
| | | | | download/ENCFF063ZLI.bigWig |
| H3K27me3 | forebrain | 11.5 | ENCFF375ONW | www.encodeproject.org/files/ENCFF375ONW/@ |
| | | | | @download/ENCFF375ONW.bigWig |
| H3K27me3 | forebrain | 12.5 | ENCFF597HKP | www.encodeproject.org/files/ENCFF597HKP/@ |
| | | | | @download/ENCFF597HKP.bigWig |
| H3K27me3 | forebrain | 13.5 | ENCFF760VJL | www.encodeproject.org/files/ENCFF760VJL/@@ |
| | | | | download/ENCFF760VJL.bigWig |
| H3K27me3 | forebrain | 14.5 | ENCFF900BKA | www.encodeproject.org/files/ENCFF900BKA/@ |
| | | | | @download/ENCFF900BKA.bigWig |
| H3K27me3 | forebrain | 15.5 | ENCFF848GOF | www.encodeproject.org/files/ENCFF848GOF/@ |
| | | | | @download/ENCFF848GOF.bigWig |
| H3K27me3 | forebrain | 16.5 | ENCFF935NKI | www.encodeproject.org/files/ENCFF935NKI/@@ |
| | | | | download/ENCFF935NKI.bigWig |
| H3K27me3 | forebrain | 20 | ENCFF183CXT | www.encodeproject.org/files/ENCFF183CXT/@ |
| | | | | @download/ENCFF183CXT.bigWig |
| H3K9me3 | forebrain | 10.5 | ENCFF075BNF | www.encodeproject.org/files/ENCFF075BNF/@ |
| | | | | @download/ENCFF075BNF.bigWig |
| H3K9me3 | forebrain | 11.5 | ENCFF052RHA | www.encodeproject.org/files/ENCFF052RHA/@ |
| | | | | @download/ENCFF052RHA.bigWig |
| H3K9me3 | forebrain | 12.5 | ENCFF137LNV | www.encodeproject.org/files/ENCFF137LNV/@ |
| | | | | @download/ENCFF137LNV.bigWig |
| H3K9me3 | forebrain | 13.5 | ENCFF101ASP | www.encodeproject.org/files/ENCFF101ASP/@ |
| | | | | @download/ENCFF101ASP.bigWig |
| H3K9me3 | forebrain | 14.5 | ENCFF034URR | www.encodeproject.org/files/ENCFF034URR/@ |
| | | | | @download/ENCFF034URR.bigWig |
| H3K9me3 | forebrain | 15.5 | ENCFF718RMZ | www.encodeproject.org/files/ENCFF718RMZ/@ |
| | | | | @download/ENCFF718RMZ.bigWig |
| H3K9me3 | forebrain | 16.5 | ENCFF809PQF | www.encodeproject.org/files/ENCFF809PQF/@ |
| | | | | @download/ENCFF809PQF.bigWig |
| H3K9me3 | forebrain | 20 | ENCFF114FKU | www.encodeproject.org/files/ENCFF114FKU/@ |
| | | | | @download/ENCFF114FKU.bigWig |
| H3K27ac | hindbrain | 10.5 | ENCFF782RAO | www.encodeproject.org/files/ENCFF782RAO/@ |
| | | | | @download/ENCFF782RAO.bigWig |
| H3K27ac | hindbrain | 11.5 | ENCFF177SVC | www.encodeproject.org/files/ENCFF177SVC/@ |
| | | | | @download/ENCFF177SVC.bigWig |
| H3K27ac | hindbrain | 12.5 | ENCFF631JYQ | www.encodeproject.org/files/ENCFF631JYQ/@ |
| | | | | @download/ENCFF631JYQ.bigWig |
| H3K27ac | hindbrain | 13.5 | ENCFF923TVC | www.encodeproject.org/files/ENCFF923TVC/@ |
| | | | | @download/ENCFF923TVC.bigWig |
| H3K27ac | hindbrain | 14.5 | ENCFF774AQE | www.encodeproject.org/files/ENCFF774AQE/@ |
| | | | | @download/ENCFF774AQE.bigWig |
| H3K27ac | hindbrain | 15.5 | ENCFF121XJR | www.encodeproject.org/files/ENCFF121XJR/@ |
| | | | | @download/ENCFF121XJR.bigWig |
| H3K27ac | hindbrain | 16.5 | ENCFF011VMX | www.encodeproject.org/files/ENCFF011VMX/@ |
| | | | | @download/ENCFF011VMX.bigWig |
| H3K27ac | hindbrain | 20 | ENCFF665WCP | www.encodeproject.org/files/ENCFF665WCP/@ |
| | | | | @download/ENCFF665WCP.bigWig |
| H3K27me3 | hindbrain | 10.5 | ENCFF898VDV | www.encodeproject.org/files/ENCFF898VDV/@ |
| | | | | @download/ENCFF898VDV.bigWig |
| H3K27me3 | hindbrain | 11.5 | ENCFF352EVI | www.encodeproject.org/files/ENCFF352EVI/@@ |
| | | | | download/ENCFF352EVI.bigWig |
| H3K27me3 | hindbrain | 12.5 | ENCFF349EPV | www.encodeproject.org/files/ENCFF349EPV/@ |
| | | | | @download/ENCFF349EPV.bigWig |
| H3K27me3 | hindbrain | 13.5 | ENCFF637PDW | www.encodeproject.org/files/ENCFF637PDW/@ |
| | | | | @download/ENCFF637PDW.bigWig |
| H3K27me3 | hindbrain | 14.5 | ENCFF787DNX | www.encodeproject.org/files/ENCFF787DNX/@ |
| | | | | @download/ENCFF787DNX.bigWig |
| H3K27me3 | hindbrain | 15.5 | ENCFF408PKX | www.encodeproject.org/files/ENCFF408PKX/@ |
| | | | | @download/ENCFF408PKX.bigWig |
| H3K27me3 | hindbrain | 16.5 | ENCFF574LGA | www.encodeproject.org/files/ENCFF574LGA/@ |
| | | | | @download/ENCFF574LGA.bigWig |
| H3K27me3 | hindbrain | 20 | ENCFF156SIF | www.encodeproject.org/files/ENCFF156SIF/@@ |
| | | | | download/ENCFFI56SIF.bigWig |
| H3K9me3 | hindbrain | 10.5 | ENCFF672EXE | www.encodeproject.org/files/ENCFF672EXE/@ |
| | | | | @download/ENCFF672EXE.bigWig |
| H3K9me3 | hindbrain | 11.5 | ENCFF570FAF | www.encodeproject.org/files/ENCFF570FAF/@ |
| | | | | @download/ENCFF570FAF.bigWig |
| H3K9me3 | hindbrain | 12.5 | ENCFF063VTT | www.encodeproject.org/files/ENCFF063VTT/@ |
| | | | | @download/ENCFF063VTT.bigWig |
| H3K9me3 | hindbrain | 13.5 | ENCFF779TQD | www.encodeproject.org/files/ENCFF779TQD/@ |
| | | | | @download/ENCFF779TQD.bigWig |
| H3K9me3 | hindbrain | 14.5 | ENCFF463QJN | www.encodeproject.org/files/ENCFF463QJN/@ |
| | | | | @download/ENCFF463QJN.bigWig |
| H3K9me3 | hindbrain | 15.5 | ENCFF499LUF | www.encodeproject.org/files/ENCFF499LUF/@ |
| | | | | @download/ENCFF499LUF.bigWig |
| H3K9me3 | hindbrain | 16.5 | ENCFF637BYB | www.encodeproject.org/files/ENCFF637BYB/@ |
| | | | | @download/ENCFF637BYB.bigWig |
| H3K9me3 | hindbrain | 20 | ENCFF919CSS | www.encodeproject.org/files/ENCFF919CSS/@ |
| | | | | @download/ENCFF919CSS.bigWig |
| H3K27ac | midbrain | 10.5 | ENCFF773NIS | www.encodeproject.org/files/ENCFF773NIS/@@ |
| | | | | download/ENCFF773NIS.bigWig |
| H3K27ac | midbrain | 11.5 | ENCFF230FQZ | www.encodeproject.org/files/ENCFF230FQZ/@ |
| | | | | @download/ENCFF230FQZ.bigWig |
| H3K27ac | midbrain | 12.5 | ENCFF406MDW | www.encodeproject.org/files/ENCFF406MDW/@ |
| | | | | @download/ENCFF406MDW.bigWig |
| H3K27ac | midbrain | 13.5 | ENCFF416UPA | www.encodeproject.org/files/ENCFF416UPA/@ |
| | | | | @download/ENCFF416UPA.bigWig |
| H3K27ac | midbrain | 14.5 | ENCFF900XUW | www.encodeproject.org/files/ENCFF900XUW/@ |
| | | | | @download/ENCFF900XUW.bigWig |
| H3K27ac | midbrain | 15.5 | ENCFF616CNP | www.encodeproject.org/files/ENCFF616CNP/@ |
| | | | | @download/ENCFF616CNP.bigWig |
| H3K27ac | midbrain | 16.5 | ENCFF447LGJ | www.encodeproject.org/files/ENCFF447LGJ/@ |
| | | | | @download/ENCFF447LGJ.bigWig |
| H3K27ac | midbrain | 20 | ENCFF961KLD | www.encodeproject.org/files/ENCFF961KLD/@ |
| | | | | @download/ENCFF961KLD.bigWig |
| H3K27me3 | midbrain | 10.5 | ENCFF293RMV | www.encodeproject.org/files/ENCFF293RMV/@ |
| | | | | @download/ENCFF293RMV.bigWig |
| H3K27me3 | midbrain | 11.5 | ENCFF485WCD | www.encodeproject.org/files/ENCFF485WCD/@ |
| | | | | @download/ENCFF485WCD.bigWig |
| H3K27me3 | midbrain | 12.5 | ENCFF612RDE | www.encodeproject.org/files/ENCFF612RDE/@ |
| | | | | @download/ENCFF612RDE.bigWig |
| H3K27me3 | midbrain | 13.5 | ENCFF610HBD | www.encodeproject.org/files/ENCFF610HBD/@ |
| | | | | @download/ENCFF610HBD.bigWig |
| H3K27me3 | midbrain | 14.5 | ENCFF908XOQ | www.encodeproject.org/files/ENCFF908XOQ/@ |
| | | | | @download/ENCFF908XOQ.bigWig |
| H3K27me3 | midbrain | 15.5 | ENCFF376ZJO | www.encodeproject.org/files/ENCFF376ZJO/@ |
| | | | | @download/ENCFF376ZJO.bigWig |
| H3K27me3 | midbrain | 16.5 | ENCFF025FNF | www.encodeproject.org/files/ENCFF025FNF/@ |
| | | | | @download/ENCFF025FNF.bigWig |
| H3K27me3 | midbrain | 20 | ENCFF149AGA | www.encodeproject.org/files/ENCFF149AGA/@ |
| | | | | @download/ENCFF149AGA.bigWig |
| H3K9me3 | midbrain | 10.5 | ENCFF190AYI | www.encodeproject.org/files/ENCFF190AYI/@@ |
| | | | | download/ENCFF190AYI.bigWig |
| H3K9me3 | midbrain | 11.5 | ENCFF402XZO | www.encodeproject.org/files/ENCFF402XZO/@ |
| | | | | @download/ENCFF402XZO.bigWig |
| H3K9me3 | midbrain | 12.5 | ENCFF432ADZ | www.encodeproject.org/files/ENCFF432ADZ/@ |
| | | | | @download/ENCFF432ADZ.bigWig |
| H3K9me3 | midbrain | 13.5 | ENCFF878NXY | www.encodeproject.org/files/ENCFF878NXY/@ |
| | | | | @download/ENCFF878NXY.bigWig |
| H3K9me3 | midbrain | 14.5 | ENCFF966CDF | www.encodeproject.org/files/ENCFF966CDF/@ |
| | | | | @download/ENCFF966CDF.bigWig |
| H3K9me3 | midbrain | 15.5 | ENCFF377LTJ | www.encodeproject.org/files/ENCFF377LTJ/@@ |
| | | | | download/ENCFF377LTJ.bigWig |
| H3K9me3 | midbrain | 16.5 | ENCFF390PNF | www.encodeproject.org/files/ENCFF390PNF/@ |
| | | | | @download/ENCFF390PNF.bigWig |
| H3K9me3 | midbrain | 20 | ENCFF887HCS | www.encodeproject.org/files/ENCFF887HCS/@ |
| | | | | @download/ENCFF887HCS.bigWig |
| H3K27ac | neuralTube | 11.5 | ENCFF422NJA | www.encodeproject.org/files/ENCFF422NJA/@ |
| | | | | @download/ENCFF422NJA.bigWig |
| H3K27ac | neuralTube | 12.5 | ENCFF704GSX | www.encodeproject.org/files/ENCFF704GSX/@ |
| | | | | @download/ENCFF704GSX.bigWig |
| H3K27ac | neuralTube | 13.5 | ENCFF066PYN | www.encodeproject.org/files/ENCFF066PYN/@ |
| | | | | @download/ENCFF066PYN.bigWig |
| H3K27ac | neuralTube | 14.5 | ENCFF494OOQ | www.encodeproject.org/files/ENCFF494OOQ/@ |
| | | | | @download/ENCFF494OOQ.bigWig |
| H3K27ac | neuralTube | 15.5 | ENCFF789UMC | www.encodeproject.org/files/ENCFF789UMC/@ |
| | | | | @download/ENCFF789UMC.bigWig |
| H3K27me3 | neuralTube | 11.5 | ENCFF217EPP | www.encodeproject.org/files/ENCFF217EPP/@ |
| | | | | @download/ENCFF217EPP.bigWig |
| H3K27me3 | neuralTube | 12.5 | ENCFF901MQW | www.encodeproject.org/files/ENCFF901MQW/@ |
| | | | | @download/ENCFF901MQW.bigWig |
| H3K27me3 | neuralTube | 13.5 | ENCFF009QET | www.encodeproject.org/files/ENCFF009QET/@ |
| | | | | @download/ENCFF009QET.bigWig |
| H3K27me3 | neuralTube | 14.5 | ENCFF223PHS | www.encodeproject.org/files/ENCFF223PHS/@ |
| | | | | @download/ENCFF223PHS.bigWig |
| H3K27me3 | neuralTube | 15.5 | ENCFF386EYG | www.encodeproject.org/files/ENCFF386EYG/@ |
| | | | | @download/ENCFF386EYG.bigWig |
| H3K9me3 | neuralTube | 11.5 | ENCFF973ZKZ | www.encodeproject.org/files/ENCFF973ZKZ/@ |
| | | | | @download/ENCFF973ZKZ.bigWig |
| H3K9me3 | neuralTube | 12.5 | ENCFF197JHX | www.encodeproject.org/files/ENCFF197JHX/@ |
| | | | | @download/ENCFF197JHX.bigWig |
| H3K9me3 | neuralTube | 13.5 | ENCFF495VRT | www.encodeproject.org/files/ENCFF495VRT/@ |
| | | | | @download/ENCFF495VRT.bigWig |
| H3K9me3 | neuralTube | 14.5 | ENCFF705MSQ | www.encodeproject.org/files/ENCFF705MSQ/@ |
| | | | | @download/ENCFF705MSQ.bigWig |
| H3K9me3 | neuralTube | 15.5 | ENCFF987UOT | www.encodeproject.org/files/ENCFF987UOT/@ |
| | | | | @download/ENCFF987UOT.bigWig |
| H3K27ac | intestine | 14.5 | ENCFF251XZW | www.encodeproject.org/files/ENCFF251XZW/@ |
| | | | | @download/ENCFF251XZW.bigWig |
| H3K27ac | intestine | 15.5 | ENCFF803SVJ | www.encodeproject.org/files/ENCFF803SVJ/@ |
| | | | | @download/ENCFF803SVJ.bigWig |
| H3K27ac | intestine | 16.5 | ENCFF829GXB | www.encodeproject.org/files/ENCFF829GXB/@ |
| | | | | @download/ENCFF829GXB.bigWig |
| H3K27ac | intestine | 20 | ENCFF512TCI | www.encodeproject.org/files/ENCFF512TCI/@@ |
| | | | | download/ENCFF512TCI.bigWig |
| H3K27me3 | intestine | 14.5 | ENCFF396TSB | www.encodeproject.org/files/ENCFF396TSB/@ |
| | | | | @download/ENCFF396TSB.bigWig |
| H3K27me3 | intestine | 15.5 | ENCFF270YCY | www.encodeproject.org/files/ENCFF270YCY/@ |
| | | | | @download/ENCFF270YCY.bigWig |
| H3K27me3 | intestine | 16.5 | ENCFF893IAL | www.encodeproject.org/files/ENCFF893IAL/@@ |
| | | | | download/ENCFF893IAL.bigWig |
| H3K27me3 | intestine | 20 | ENCFF133XFD | www.encodeproject.org/files/ENCFF133XFD/@ |
| | | | | @download/ENCFF133XFD.bigWig |
| H3K9me3 | intestine | 14.5 | ENCFF814OZR | www.encodeproject.org/files/ENCFF814OZR/@ |
| | | | | @download/ENCFF814OZR.bigWig |
| H3K9me3 | intestine | 15.5 | ENCFF776ZXU | www.encodeproject.org/files/ENCFF776ZXU/@ |
| | | | | @download/ENCFF776ZXU.bigWig |
| H3K9me3 | intestine | 16.5 | ENCFF981JRQ | www.encodeproject.org/files/ENCFF981JRQ/@ |
| | | | | @download/ENCFF981JRQ.bigWig |
| H3K9me3 | intestine | 20 | ENCFF363IUI | www.encodeproject.org/files/ENCFF363IUI/@@ |
| | | | | download/ENCFF363IUI.bigWig |
| H3K27ac | stomach | 14.5 | ENCFF036PPT | www.encodeproject.org/files/ENCFF036PPT/@ |
| | | | | @download/ENCFF036PPT.bigWig |
| H3K27ac | stomach | 15.5 | ENCFF604BOY | www.encodeproject.org/files/ENCFF604BOY/@ |
| | | | | @download/ENCFF604BOY.bigWig |
| H3K27ac | stomach | 16.5 | ENCFF069OMF | www.encodeproject.org/files/ENCFF069OMF/@ |
| | | | | @download/ENCFF069OMF.bigWig |
| H3K27ac | stomach | 20 | ENCFF224LDB | www.encodeproject.org/files/ENCFF224LDB/@ |
| | | | | @download/ENCFF224LDB.bigWig |
| H3K27me3 | stomach | 14.5 | ENCFF518FTX | www.encodeproject.org/files/ENCFF518FTX/@ |
| | | | | @download/ENCFF518FTX.bigWig |
| H3K27me3 | stomach | 15.5 | ENCFF886OPQ | www.encodeproject.org/files/ENCFF886OPQ/@ |
| | | | | @download/ENCFF886OPQ.bigWig |
| H3K27me3 | stomach | 16.5 | ENCFF039QOO | www.encodeproject.org/files/ENCFF039QOO/@ |
| | | | | @download/ENCFF039QOO. bigWig |
| H3K27me3 | stomach | 20 | ENCFF587CHP | www.encodeproject.org/files/ENCFF587CHP/@ |
| | | | | @download/ENCFF587CHP.bigWig |
| H3K9me3 | stomach | 14.5 | ENCFF968CFU | www.encodeproject.org/files/ENCFF968CFU/@ |
| | | | | @download/ENCFF968CFU.bigWig |
| H3K9me3 | stomach | 15.5 | ENCFF417JCK | www.encodeproject.org/files/ENCFF417JCK/@ |
| | | | | @download/ENCFF417JCK.bigWig |
| H3K9me3 | stomach | 16.5 | ENCFF824UJK | www.encodeproject.org/files/ENCFF824UJK/@ |
| | | | | @download/ENCFF824UJK.bigWig |
| H3K9me3 | stomach | 20 | ENCFF591NXE | www.encodeproject.org/files/ENCFF591NXE/@ |
| | | | | @download/ENCFF591NXE.bigWig |
| H3K27ac | liver | 11.5 | ENCFF274AWM | www.encodeproject.org/files/ENCFF274AWM/@ |
| | | | | @download/ENCFF274AWM.bigWig |
| H3K27ac | liver | 12.5 | ENCFF285ZJD | www.encodeproject.org/files/ENCFF285ZJD/@ |
| | | | | @download/ENCFF285ZJD.bigWig |
| H3K27ac | liver | 13.5 | ENCFF289PYS | www.encodeproject.org/files/ENCFF289PYS/@ |
| | | | | @download/ENCFF289PYS.bigWig |
| H3K27ac | liver | 14.5 | ENCFF518VSR | www.encodeproject.org/files/ENCFF518VSR/@ |
| | | | | @download/ENCFF518VSR.bigWig |
| H3K27ac | liver | 15.5 | ENCFF290NHP | www.encodeproject.org/files/ENCFF290NHP/@ |
| | | | | @download/ENCFF290NHP.bigWig |
| H3K27ac | liver | 16.5 | ENCFF526VAG | www.encodeproject.org/files/ENCFF526VAG/@ |
| | | | | @download/ENCFF526VAG.bigWig |
| H3K27ac | liver | 20 | ENCFF588AKC | www.encodeproject.org/files/ENCFF588AKC/@ |
| | | | | @download/ENCFF588AKC.bigWig |
| H3K27ac | liver | 56 | ENCFF632VNQ | www.encodeproject.org/files/ENCFF632VNQ/@ |
| | | | | @download/ENCFF632VNQ.bigWig |
| H3K27me3 | liver | 11.5 | ENCFF800NSG | www.encodeproject.org/files/ENCFF800NSG/@ |
| | | | | @download/ENCFF800NSG.bigWig |
| H3K27me3 | liver | 12.5 | ENCFF361SUJ | www.encodeproject.org/files/ENCFF361SUJ/@ |
| | | | | @download/ENCFF361SUJ.bigWig |
| H3K27me3 | liver | 13.5 | ENCFF914LRA | www.encodeproject.org/files/ENCFF914LRA/@ |
| | | | | @download/ENCFF914LRA.bigWig |
| H3K27me3 | liver | 14.5 | ENCFF035UHY | www.encodeproject.org/files/ENCFF035UHY/@ |
| | | | | @download/ENCFF035UHY.bigWig |
| H3K27me3 | liver | 15.5 | ENCFF520CMB | www.encodeproject.org/files/ENCFF520CMB/@ |
| | | | | @download/ENCFF520CMB.bigWig |
| H3K27me3 | liver | 16.5 | ENCFF943PON | www.encodeproject.org/files/ENCFF943PON/@ |
| | | | | @download/ENCFF943PON.bigWig |
| H3K27me3 | liver | 20 | ENCFF633GEF | www.encodeproject.org/files/ENCFF633GEF/@ |
| | | | | @download/ENCFF633GEF.bigWig |
| H3K27me3 | liver | 56 | ENCFF172GJQ | www.encodeproject.org/files/ENCFF172GJQ/@ |
| | | | | @download/ENCFF172GJQ.bigWig |
| H3K9me3 | liver | 11.5 | ENCFF477UFW | www.encodeproject.org/files/ENCFF477UFW/@ |
| | | | | @download/ENCFF477UFW.bigWig |
| H3K9me3 | liver | 12.5 | ENCFF741LSB | www.encodeproject.org/files/ENCFF741LSB/@ |
| | | | | @download/ENCFF741LSB.bigWig |
| H3K9me3 | liver | 13.5 | ENCFF258AAD | www.encodeproject.org/files/ENCFF258AAD/@ |
| | | | | @download/ENCFF258AAD.bigWig |
| H3K9me3 | liver | 14.5 | ENCFF716TGN | www.encodeproject.org/files/ENCFF716TGN/@ |
| | | | | @download/ENCFF716TGN.bigWig |
| H3K9me3 | liver | 15.5 | ENCFF550PJQ | www.encodeproject.org/files/ENCFF550PJQ/@ |
| | | | | @download/ENCFF550PJQ.bigWig |
| H3K9me3 | liver | 16.5 | ENCFF947IGN | www.encodeproject.org/files/ENCFF947IGN/@ |
| | | | | @download/ENCFF947IGN.bigWig |
| H3K9me3 | liver | 20 | ENCFF040LFN | www.encodeproject.org/files/ENCFF040LFN/@ |
| | | | | @download/ENCFF040LFN.bigWig |
| H3K27ac | kidney | 14.5 | ENCFF298GLI | www.encodeproject.org/files/ENCFF298GLI/@@ |
| | | | | download/ENCFF298GLI.bigWig |
| H3K27ac | kidney | 15.5 | ENCFF297RMH | www.encodeproject.org/files/ENCFF297RMH/@ |
| | | | | @download/ENCFF297RMH.bigWig |
| H3K27ac | kidney | 16.5 | ENCFF132TLS | www.encodeproject.org/files/ENCFF132TLS/@ |
| | | | | @download/ENCFF132TLS.bigWig |
| H3K27ac | kidney | 20 | ENCFF873EBR | www.encodeproject.org/files/ENCFF873EBR/@ |
| | | | | @download/ENCFF873EBR.bigWig |
| H3K27ac | kidney | 56 | ENCFF188DKO | www.encodeproject.org/files/ENCFF188DKO/@ |
| | | | | @download/ENCFF188DKO.bigWig |
| H3K27me3 | kidney | 14.5 | ENCFF324UGW | www.encodeproject.org/files/ENCFF324UGW/@ |
| | | | | @download/ENCFF324UGW.bigWig |
| H3K27me3 | kidney | 15.5 | ENCFF201DBX | www.encodeproject.org/files/ENCFF201DBX/@ |
| | | | | @download/ENCFF201DBX.bigWig |
| H3K27me3 | kidney | 16.5 | ENCFF089CJB | www.encodeproject.org/files/ENCFF089CJB/@ |
| | | | | @download/ENCFF089CJB.bigWig |
| H3K27me3 | kidney | 20 | ENCFF460ZYC | www.encodeproject.org/files/ENCFF460ZYC/@ |
| | | | | @download/ENCFF460ZYC.bigWig |
| H3K27me3 | kidney | 56 | ENCFF324KCF | www.encodeproject.org/files/ENCFF324KCF/@ |
| | | | | @download/ENCFF324KCF.bigWig |
| H3K9me3 | kidney | 14.5 | ENCFF433RSJ | www.encodeproject.org/files/ENCFF433RSJ/@ |
| | | | | @download/ENCFF433RSJ.bigWig |
| H3K9me3 | kidney | 15.5 | ENCFF209XEZ | www.encodeproject.org/files/ENCFF209XEZ/@ |
| | | | | @download/ENCFF209XEZ.bigWig |
| H3K9me3 | kidney | 16.5 | ENCFF767LND | www.encodeproject.org/files/ENCFF767LND/@ |
| | | | | @download/ENCFF767LND.bigWig |
| H3K9me3 | kidney | 20 | ENCFF517QXW | www.encodeproject.org/files/ENCFF517QXW/@ |
| | | | | @download/ENCFF517QXW.bigWig |
| H3K27ac | heart | 10.5 | ENCFF222TEU | www.encodeproject.org/files/ENCFF222TEU/@ |
| | | | | @download/ENCFF222TEU.bigWig |
| H3K27ac | heart | 11.5 | ENCFF305IHU | www.encodeproject.org/files/ENCFF305IHU/@@ |
| | | | | download/ENCFF305IHU.bigWig |
| H3K27ac | heart | 12.5 | ENCFF937AXD | www.encodeproject.org/files/ENCFF937AXD/@ |
| | | | | @download/ENCFF937AXD.bigWig |
| H3K27ac | heart | 13.5 | ENCFF870SQY | www.encodeproject.org/files/ENCFF870SQY/@ |
| | | | | @download/ENCFF870SQY.bigWig |
| H3K27ac | heart | 14.5 | ENCFF689PCR | www.encodeproject.org/files/ENCFF689PCR/@ |
| | | | | @download/ENCFF689PCR.bigWig |
| H3K27ac | heart | 15.5 | ENCFF021RTZ | www.encodeproject.org/files/ENCFF021RTZ/@ |
| | | | | @download/ENCFF021RTZ.bigWig |
| H3K27ac | heart | 16.5 | ENCFF801FFG | www.encodeproject.org/files/ENCFF801FFG/@ |
| | | | | @download/ENCFF801FFG.bigWig |
| H3K27ac | heart | 20 | ENCFF804EYG | www.encodeproject.org/files/ENCFF804EYG/@ |
| | | | | @download/ENCFF804EYG.bigWig |
| H3K27ac | heart | 56 | ENCFF691YDA | www.encodeproject.org/files/ENCFF691YDA/@ |
| | | | | @download/ENCFF691YDA.bigWig |
| H3K27me3 | heart | 10.5 | ENCFF046VWN | www.encodeproject.org/files/ENCFF046VWN/@ |
| | | | | @download/ENCFF046VWN.bigWig |
| H3K27me3 | heart | 11.5 | ENCFF551QTH | www.encodeproject.org/files/ENCFF551QTH/@ |
| | | | | @download/ENCFF551QTH.bigWig |
| H3K27me3 | heart | 12.5 | ENCFF379MVJ | www.encodeproject.org/files/ENCFF379MVJ/@ |
| | | | | @download/ENCFF379MVJ.bigWig |
| H3K27me3 | heart | 13.5 | ENCFF972QDT | www.encodeproject.org/files/ENCFF972QDT/@ |
| | | | | @download/ENCFF972QDT.bigWig |
| H3K27me3 | heart | 14.5 | ENCFF129FZH | www.encodeproject.org/files/ENCFF129FZH/@ |
| | | | | @download/ENCFF129FZH.bigWig |
| H3K27me3 | heart | 15.5 | ENCFF044FTG | www.encodeproject.org/files/ENCFF044FTG/@ |
| | | | | @download/ENCFF044FTG.bigWig |
| H3K27me3 | heart | 16.5 | ENCFF280UTR | www.encodeproject.org/files/ENCFF280UTR/@ |
| | | | | @download/ENCFF280UTR.bigWig |
| H3K27me3 | heart | 20 | ENCFF845OXU | www.encodeproject.org/files/ENCFF845OXU/@ |
| | | | | @download/ENCFF845OXU.bigWig |
| H3K27me3 | heart | 56 | ENCFF344SFV | www.encodeproject.org/files/ENCFF344SFV/@ |
| | | | | @download/ENCFF344SFV.bigWig |
| H3K9me3 | heart | 10.5 | ENCFF172OCI | www.encodeproject.org/files/ENCFF172OCI/@ |
| | | | | @download/ENCFF172OCI.bigWig |
| H3K9me3 | heart | 11.5 | ENCFF207EPW | www.encodeproject.org/files/ENCFF207EPW/@ |
| | | | | @download/ENCFF207EPW.bigWig |
| H3K9me3 | heart | 12.5 | ENCFF797OAF | www.encodeproject.org/files/ENCFF797OAF/@ |
| | | | | @download/ENCFF797OAF.bigWig |
| H3K9me3 | heart | 13.5 | ENCFF637INN | www.encodeproject.org/files/ENCFF637INN/@@ |
| | | | | download/ENCFF637INN.bigWig |
| H3K9me3 | heart | 14.5 | ENCFF409BJZ | www.encodeproject.org/files/ENCFF409BJZ/@ |
| | | | | @download/ENCFF409BJZ.bigWig |
| H3K9me3 | heart | 15.5 | ENCFF921YZN | www.encodeproject.org/files/ENCFF921YZN/@ |
| | | | | @download/ENCFF921YZN.bigWig |
| H3K9me3 | heart | 16.5 | ENCFF637DCW | www.encodeproject.org/files/ENCFF637DCW/@ |
| | | | | @download/ENCFF637DCW.bigWig |
| H3K9me3 | heart | 20 | ENCFF046SEF | www.encodeproject.org/files/ENCFF046SEF/@ |
| | | | | @download/ENCFF046SEF.bigWig |
| H3K27ac | lung | 14.5 | ENCFF173XOV | www.encodeproject.org/files/ENCFF173XOV/@ |
| | | | | @download/ENCFF173XOV.bigWig |
| H3K27ac | lung | 15.5 | ENCFF018LWM | www.encodeproject.org/files/ENCFF018LWM/@ |
| | | | | @download/ENCFF018LWM.bigWig |
| H3K27ac | lung | 16.5 | ENCFF681GAF | www.encodeproject.org/files/ENCFF681GAF/@ |
| | | | | @download/ENCFF681GAF.bigWig |
| H3K27ac | lung | 20 | ENCFF924XGB | www.encodeproject.org/files/ENCFF924XGB/@ |
| | | | | @download/ENCFF924XGB.bigWig |
| H3K27me3 | lung | 14.5 | ENCFF164GUC | www.encodeproject.org/files/ENCFF164GUC/@ |
| | | | | @download/ENCFF164GUC.bigWig |
| H3K27me3 | lung | 15.5 | ENCFF024LWP | www.encodeproject.org/files/ENCFF024LWP/@ |
| | | | | @download/ENCFF024LWP.bigWig |
| H3K27me3 | lung | 16.5 | ENCFF749LHU | www.encodeproject.org/files/ENCFF749LHU/@ |
| | | | | @download/ENCFF749LHU.bigWig |
| H3K27me3 | lung | 20 | ENCFF210YRR | www.encodeproject.org/files/ENCFF210YRR/@ |
| | | | | @download/ENCFF210YRR.bigWig |
| H3K9me3 | lung | 14.5 | ENCFF492VCK | www.encodeproject.org/files/ENCFF492VCK/@ |
| | | | | @download/ENCFF492VCK.bigWig |
| H3K9me3 | lung | 15.5 | ENCFF368BVK | www.encodeproject.org/files/ENCFF368BVK/@ |
| | | | | @download/ENCFF368BVK.bigWig |
| H3K9me3 | lung | 16.5 | ENCFF828TZR | www.encodeproject.org/files/ENCFF828TZR/@ |
| | | | | @download/ENCFF828TZR.bigWig |
| H3K9me3 | lung | 20 | ENCFF399KQJ | www.encodeproject.org/files/ENCFF399KQJ/@ |
| | | | | @download/ENCFF399KQJ.bigWig |
| H3K27ac | limb | 11.5 | ENCFF897QOI | www.encodeproject.org/files/ENCFF897QOI/@ |
| | | | | @download/ENCFF897QOI.bigWig |
| H3K27ac | limb | 12.5 | ENCFF008XYM | www.encodeproject.org/files/ENCFF008XYM/@ |
| | | | | @download/ENCFF008XYM.bigWig |
| H3K27ac | limb | 13.5 | ENCFF402FVP | www.encodeproject.org/files/ENCFF402FVP/@ |
| | | | | @download/ENCFF402FVP.bigWig |
| H3K27ac | limb | 14.5 | ENCFF538TEQ | www.encodeproject.org/files/ENCFF538TEQ/@ |
| | | | | @download/ENCFF538TEQ.bigWig |
| H3K27ac | limb | 15.5 | ENCFF199SCG | www.encodeproject.org/files/ENCFF199SCG/@ |
| | | | | @download/ENCFF199SCG.bigWig |
| H3K27me3 | limb | 11.5 | ENCFF731BDW | www.encodeproject.org/files/ENCFF731BDW/@ |
| | | | | @download/ENCFF731BDW.bigWig |
| H3K27me3 | limb | 12.5 | ENCFF865VFL | www.encodeproject.org/files/ENCFF865VFL/@ |
| | | | | @download/ENCFF865VFL.bigWig |
| H3K27me3 | limb | 13.5 | ENCFF779RNO | www.encodeproject.org/files/ENCFF779RNO/@ |
| | | | | @download/ENCFF779RNO.bigWig |
| H3K27me3 | limb | 14.5 | ENCFF014HTI | www.encodeproject.org/files/ENCFF014HTI/@@ |
| | | | | download/ENCFF014HTI.bigWig |
| H3K27me3 | limb | 15.5 | ENCFF755WDT | www.encodeproject.org/files/ENCFF755WDT/@ |
| | | | | @download/ENCFF755WDT.bigWig |
| H3K9me3 | limb | 11.5 | ENCFF937IQZ | www.encodeproject.org/files/ENCFF937IQZ/@@ |
| | | | | download/ENCFF937IQZ.bigWig |
| H3K9me3 | limb | 12.5 | ENCFF951TKF | www.encodeproject.org/files/ENCFF951TKF/@ |
| | | | | @download/ENCFF951TKF.bigWig |
| H3K9me3 | limb | 13.5 | ENCFF577WZP | www.encodeproject.org/files/ENCFF577WZP/@ |
| | | | | @download/ENCFF577WZP.bigWig |
| H3K9me3 | limb | 14.5 | ENCFF033IOU | www.encodeproject.org/files/ENCFF033IOU/@ |
| | | | | @download/ENCFF033IOU.bigWig |
| H3K9me3 | limb | 15.5 | ENCFF367TZB | www.encodeproject.org/files/ENCFF367TZB/@ |
| | | | | @download/ENCFF367TZB.bigWig |
| H3K27ac | facial | 10.5 | ENCFF440LYR | www.encodeproject.org/files/ENCFF440LYR/@ |
| | | | | @download/ENCFF440LYR.bigWig |
| H3K27ac | facial | 11.5 | ENCFF089IXS | www.encodeproject.org/files/ENCFF089IXS/@@ |
| | | | | download/ENCFF089IXS.bigWig |
| H3K27ac | facial | 12.5 | ENCFF596BHZ | www.encodeproject.org/files/ENCFF596BHZ/@ |
| | | | | @download/ENCFF596BHZ.bigWig |
| H3K27ac | facial | 13.5 | ENCFF306YHS | www.encodeproject.org/files/ENCFF306YHS/@ |
| | | | | @download/ENCFF306YHS.bigWig |
| H3K27ac | facial | 14.5 | ENCFF071HGW | www.encodeproject.org/files/ENCFF071HGW/@ |
| | | | | @download/ENCFF071HGW.bigWig |
| H3K27ac | facial | 15.5 | ENCFF033PTH | www.encodeproject.org/files/ENCFF033PTH/@ |
| | | | | @download/ENCFF033PTH.bigWig |
| H3K27me3 | facial | 10.5 | ENCFF798NZC | www.encodeproject.org/files/ENCFF798NZC/@ |
| | | | | @download/ENCFF798NZC.bigWig |
| H3K27me3 | facial | 11.5 | ENCFF422AUK | www.encodeproject.org/files/ENCFF422AUK/@ |
| | | | | @download/ENCFF422AUK.bigWig |
| H3K27me3 | facial | 12.5 | ENCFF983GFL | www.encodeproject.org/files/ENCFF983GFL/@ |
| | | | | @download/ENCFF983GFL.bigWig |
| H3K27me3 | facial | 13.5 | ENCFF672JMP | www.encodeproject.org/files/ENCFF672JMP/@ |
| | | | | @download/ENCFF672JMP.bigWig |
| H3K27me3 | facial | 14.5 | ENCFF344FSI | www.encodeproject.org/files/ENCFF344FSI/@@ |
| | | | | download/ENCFF344FSI.bigWig |
| H3K27me3 | facial | 15.5 | ENCFF944UBT | www.encodeproject.org/files/ENCFF944UBT/@ |
| | | | | @download/ENCFF944UBT.bigWig |
| H3K9me3 | facial | 10.5 | ENCFF442KVI | www.encodeproject.org/files/ENCFF442KVI/@@ |
| | | | | download/ENCFF442KVI.bigWig |
| H3K9me3 | facial | 11.5 | ENCFF505IIX | www.encodeproject.org/files/ENCFF505IIX/@@ |
| | | | | download/ENCFF505IIX.bigWig |
| H3K9me3 | facial | 12.5 | ENCFF919YGD | www.encodeproject.org/files/ENCFF919YGD/@ |
| | | | | @download/ENCFF919YGD.bigWig |
| H3K9me3 | facial | 13.5 | ENCFF421HSW | www.encodeproject.org/files/ENCFF421HSW/@ |
| | | | | @download/ENCFF421HSW.bigWig |
| H3K9me3 | facial | 14.5 | ENCFF933OKD | www.encodeproject.org/files/ENCFF933OKD/@ |
| | | | | @download/ENCFF933OKD.bigWig |
| H3K9me3 | facial | 15.5 | ENCFF820QKT | www.encodeproject.org/files/ENCFF820QKT/@ |
| | | | | @download/ENCFF820QKT.bigWig |

TABLE 11
Stratification of PRC2-bound silencers into four groups, I, II, III, and IV.

| Chr | Start | End | Group | Chr | Start | End | Group |
|---|---|---|---|---|---|---|---|
| chr3 | 88237599 | 88242602 | I | chr15 | 87543451 | 87546063 | I |
| chr3 | 88240796 | 88242456 | I | chr13 | 36724200 | 36725200 | I |
| chr7 | 79495660 | 79496915 | I | chr13 | 36723060 | 36725426 | I |
| chr2 | 152049452 | 152051243 | I | chr13 | 36723310 | 36724490 | I |
| chr2 | 152045878 | 152049156 | I | chr13 | 36723359 | 36724359 | I |
| chr7 | 140056065 | 140059407 | I | chr13 | 107646160 | 107648242 | I |
| chr7 | 140057319 | 140058701 | I | chr13 | 107644744 | 107646248 | I |
| chr4 | 22477615 | 22481565 | I | chr8 | 34395909 | 34398376 | I |
| chr4 | 22479779 | 22481611 | I | chr3 | 107534933 | 107540543 | I |
| chr4 | 22478252 | 22479329 | I | chr3 | 107536572 | 107539191 | I |
| chr4 | 22495434 | 22497568 | I | chr15 | 102734797 | 102735797 | I |
| chr4 | 22497167 | 22499410 | I | chr13 | 40606210 | 40607290 | I |
| chr4 | 22498504 | 22499504 | I | chr15 | 102735711 | 102736817 | I |
| chr7 | 79571284 | 79573281 | I | chr11 | 98338317 | 98340459 | I |
| chr7 | 79571386 | 79573917 | I | chr11 | 98336276 | 98340610 | I |
| chr14 | 122481280 | 122487381 | I | chr2 | 33542949 | 33548237 | I |
| chr9 | 91354259 | 91356307 | I | chr2 | 33542407 | 33547238 | I |
| chr9 | 91354882 | 91356407 | I | chr2 | 33542631 | 33546242 | I |
| chr9 | 91353458 | 91354923 | I | chr16 | 17833458 | 17834937 | I |
| chr14 | 122406139 | 122409107 | I | chr16 | 17833419 | 17834419 | I |
| chr14 | 122404897 | 122407214 | I | chr16 | 17833603 | 17835252 | I |
| chr14 | 122406492 | 122407842 | I | chr15 | 85623423 | 85627179 | I |
| chr14 | 122405894 | 122407296 | I | chr15 | 85625143 | 85626359 | I |
| chr13 | 83743506 | 83744644 | I | chr2 | 61815014 | 61816023 | I |
| chr13 | 83714136 | 83715976 | I | chr10 | 80208743 | 80209899 | I |
| chr5 | 120409167 | 120411493 | I | chr6 | 100562644 | 100564697 | I |
| chr5 | 120408996 | 120410747 | I | chr13 | 37311280 | 37312819 | I |
| chr9 | 37451316 | 37463581 | I | chr13 | 37311334 | 37313551 | I |
| chr9 | 37455214 | 37463261 | I | chr10 | 59955224 | 59957816 | I |
| chr18 | 76533140 | 76535698 | I | chr10 | 59956376 | 59958113 | I |
| chr9 | 37458935 | 37460874 | I | chr17 | 25555715 | 25557734 | I |
| chr9 | 37458466 | 37461526 | I | chr7 | 44428161 | 44431049 | I |
| chr9 | 37457887 | 37461983 | I | chr7 | 44425297 | 44431388 | I |
| chr9 | 37459485 | 37460488 | I | chr7 | 25177700 | 25180229 | I |
| chr9 | 37459175 | 37460601 | I | chr7 | 25178434 | 25180077 | I |
| chr10 | 110454509 | 110455509 | I | chr13 | 84344773 | 84347715 | I |
| chr4 | 107672657 | 107676006 | I | chr15 | 102745665 | 102748235 | I |
| chr7 | 65526945 | 65528097 | I | chr15 | 102745627 | 102748153 | I |
| chr7 | 65526781 | 65528108 | I | chr15 | 102746504 | 102748675 | I |
| chr7 | 78880320 | 78884392 | I | chr15 | 102745458 | 102747734 | I |
| chr7 | 78878009 | 78882819 | I | chr15 | 102745242 | 102747567 | I |
| chr13 | 54874282 | 54875945 | I | chr15 | 102747110 | 102748346 | I |
| chr15 | 102744338 | 102749614 | I | chr7 | 137317994 | 137320374 | I |
| chr15 | 102743668 | 102749147 | I | chr7 | 137318165 | 137320785 | I |
| chr15 | 102742832 | 102749181 | I | chr7 | 137317509 | 137319247 | I |
| chr15 | 102743379 | 102750411 | I | chr7 | 137321951 | 137323361 | I |
| chr15 | 102744542 | 102748391 | I | chr7 | 137321640 | 137322670 | I |
| chr15 | 102744446 | 102748239 | I | chr7 | 137320426 | 137322417 | I |
| chr15 | 102745224 | 102748663 | I | chr11 | 115370617 | 115375627 | I |
| chr11 | 119051475 | 119060400 | I | chr2 | 164938605 | 164940941 | I |
| chr10 | 110454669 | 110456423 | I | chr18 | 23309023 | 23311820 | I |
| chr11 | 119053474 | 119055550 | I | chr18 | 23309415 | 23311355 | I |
| chr11 | 119053894 | 119056314 | I | chr18 | 23310144 | 23311287 | I |
| chr6 | 51611890 | 51613085 | I | chr6 | 71010838 | 71014907 | I |
| chr13 | 84342223 | 84349697 | I | chr4 | 103619161 | 103621800 | I |
| chr13 | 84343600 | 84349253 | I | chr10 | 73097936 | 73100706 | I |
| chr2 | 61656443 | 61658061 | I | chr9 | 122567989 | 122569212 | I |
| chr2 | 61655493 | 61656884 | I | chr6 | 91842614 | 91844618 | I |
| chr2 | 61654203 | 61659205 | I | chr6 | 91842768 | 91843775 | I |
| chr10 | 127509344 | 127511306 | I | chr7 | 140060568 | 140062167 | I |
| chr15 | 99664201 | 99666374 | I | chr7 | 140060058 | 140062376 | I |
| chr15 | 99663568 | 99665440 | I | chr7 | 140060402 | 140061404 | I |
| chr15 | 99663480 | 99667403 | I | chr4 | 21688565 | 21689909 | I |
| chr18 | 47537438 | 47538445 | I | chr4 | 21688616 | 21689624 | I |
| chr2 | 168625733 | 168628354 | I | chr4 | 136861902 | 136864697 | I |
| chr5 | 149258199 | 149260019 | I | chr1 | 177322999 | 177324913 | I |
| chr11 | 118998640 | 119000545 | I | chr12 | 118853696 | 118855445 | I |
| chr11 | 118998309 | 119000661 | I | chr13 | 56208300 | 56209639 | I |
| chr11 | 118998697 | 119000345 | I | chr4 | 126619270 | 126620845 | I |
| chr11 | 118998591 | 119002038 | I | chr4 | 126619719 | 126620958 | I |
| chr11 | 118998890 | 119000769 | I | chr4 | 126619029 | 126622135 | I |
| chr11 | 118997735 | 118999450 | I | chr14 | 118231657 | 118232659 | I |
| chr5 | 139551680 | 139554926 | I | chr14 | 118230216 | 118232931 | I |
| chr5 | 139585007 | 139586007 | I | chr13 | 78182467 | 78183475 | I |
| chr5 | 139550788 | 139552340 | I | chr13 | 51916984 | 51921940 | I |
| chr5 | 139551509 | 139552519 | I | chr13 | 51918977 | 51919977 | I |
| chr14 | 118435372 | 118439845 | I | chr12 | 25124335 | 25125773 | I |
| chr14 | 118436171 | 118438186 | I | chr5 | 139598103 | 139601426 | I |
| chr8 | 92460003 | 92461646 | I | chr15 | 99092824 | 99093824 | I |
| chr8 | 92460035 | 92461958 | I | chr2 | 93179529 | 93181627 | I |
| chr8 | 92459329 | 92462073 | I | chr11 | 112871557 | 112873268 | I |
| chr8 | 92459006 | 92462381 | I | chr19 | 45540347 | 45546273 | I |
| chr11 | 84641628 | 84643767 | I | chr19 | 45539053 | 45543993 | I |
| chr11 | 84642838 | 84643838 | I | chr19 | 45540951 | 45543084 | I |
| chr11 | 84631435 | 84644718 | I | chr11 | 117027013 | 117028831 | I |
| chr13 | 97007991 | 97009514 | I | chr19 | 44881187 | 44883171 | I |
| chr6 | 88498292 | 88500513 | I | chr19 | 44878187 | 44883951 | I |
| chr2 | 115852199 | 115853199 | I | chr11 | 75290227 | 75292745 | I |
| chr2 | 115852096 | 115854082 | I | chr11 | 75289670 | 75295566 | I |
| chr2 | 115853888 | 115854890 | I | chr11 | 75292870 | 75294444 | I |
| chr19 | 45292734 | 45295019 | I | chr8 | 46214651 | 46222308 | I |
| chr13 | 71457796 | 71459697 | I | chrX | 103693893 | 103695378 | I |
| chr13 | 71457685 | 71459685 | I | chr11 | 98457408 | 98458878 | I |
| chr13 | 71457552 | 71460081 | I | chr11 | 119054653 | 119056559 | I |
| chr17 | 23699127 | 23700370 | I | chr18 | 38145930 | 38149790 | I |
| chr17 | 23699222 | 23700278 | I | chr2 | 94273198 | 94275974 | I |
| chr2 | 173585229 | 173587260 | I | chr2 | 94272512 | 94274436 | I |
| chr2 | 173584767 | 173586834 | I | chr2 | 94272087 | 94275160 | I |
| chr2 | 173585436 | 173586948 | I | chr11 | 82970617 | 82972486 | I |
| chr2 | 173585342 | 173588177 | I | chr11 | 82970348 | 82972308 | I |
| chr2 | 173584342 | 173586280 | I | chr11 | 82969767 | 82972260 | I |
| chr2 | 173585873 | 173586873 | I | chr2 | 118976022 | 118977494 | I |
| chr2 | 173582386 | 173586900 | I | chr2 | 118976299 | 118977347 | I |
| chr15 | 103168260 | 103170778 | I | chr2 | 118976604 | 118977604 | I |
| chr15 | 103168268 | 103170503 | I | chr2 | 118975606 | 118977795 | I |
| chr15 | 103168331 | 103170924 | I | chr11 | 75965616 | 75967391 | I |
| chr15 | 103168029 | 103170055 | I | chr11 | 75966085 | 75967091 | I |
| chr15 | 103168896 | 103170458 | I | chr2 | 44556525 | 44558591 | I |
| chr15 | 103167289 | 103171251 | I | chr11 | 85843067 | 85844508 | I |
| chr15 | 103168029 | 103173398 | I | chr11 | 85841963 | 85842963 | I |
| chr15 | 103085244 | 103087794 | I | chr5 | 52474634 | 52479083 | I |
| chr15 | 103085671 | 103088837 | I | chr5 | 52475067 | 52476710 | I |
| chr15 | 103085688 | 103088056 | I | chr18 | 11351189 | 11354022 | I |
| chr15 | 103084897 | 103087692 | I | chr2 | 18693435 | 18696377 | I |
| chr15 | 103085387 | 103087590 | I | chr2 | 18692071 | 18695302 | I |
| chr15 | 103084459 | 103087111 | I | chr2 | 18691212 | 18694575 | I |
| chr15 | 103084871 | 103086852 | I | chr12 | 85485390 | 85487668 | I |
| chr15 | 103084496 | 103088665 | I | chrX | 103694297 | 103696292 | I |
| chr15 | 103083799 | 103088860 | I | chr9 | 110778738 | 110782807 | I |
| chr15 | 103084763 | 103088898 | I | chr9 | 110780810 | 110781996 | I |
| chr15 | 103085348 | 103089069 | I | chr3 | 8765115 | 8769412 | I |
| chr15 | 103083620 | 103090815 | I | chr3 | 8767104 | 8768183 | I |
| chr15 | 103081143 | 103089904 | I | chr6 | 114967829 | 114970181 | I |
| chr19 | 45231030 | 45232030 | I | chr6 | 114968308 | 114970835 | I |
| chr11 | 118957059 | 118958577 | I | chr10 | 120550741 | 120554206 | I |
| chr4 | 130860639 | 130866519 | I | chr10 | 120551518 | 120553686 | I |
| chr4 | 47435307 | 47437721 | I | chr11 | 94883259 | 94884992 | I |
| chr2 | 36203064 | 36204066 | I | chr11 | 94880315 | 94882759 | I |
| chr11 | 94880086 | 94881741 | I | chr14 | 25316908 | 25319192 | I |
| chr11 | 94881617 | 94882720 | I | chr2 | 115857261 | 115859308 | I |
| chr6 | 51171759 | 51180651 | I | chr2 | 115857263 | 115858393 | I |
| chr6 | 51171019 | 51180256 | I | chr18 | 35932930 | 35935061 | I |
| chr6 | 51168120 | 51185559 | I | chr2 | 115859866 | 115860866 | I |
| chr6 | 51171757 | 51174924 | I | chr2 | 115860236 | 115861566 | I |
| chr6 | 51171574 | 51172574 | I | chr2 | 115859603 | 115861654 | I |
| chr6 | 51173151 | 51175843 | I | chr2 | 115859734 | 115860757 | I |
| chr6 | 51173180 | 51175528 | I | chr11 | 89297711 | 89298711 | I |
| chr6 | 51172963 | 51174101 | I | chr9 | 23082798 | 23084844 | I |
| chr9 | 32672531 | 32673969 | I | chrX | 99567700 | 99570776 | I |
| chr4 | 136146488 | 136147490 | I | chrX | 99566294 | 99568269 | I |
| chr17 | 83322383 | 83324166 | I | chr3 | 87958005 | 87959965 | I |
| chr17 | 47973549 | 47975323 | I | chr10 | 120271567 | 120273399 | I |
| chr15 | 67175684 | 67178096 | I | chr8 | 10922521 | 10923931 | I |
| chr15 | 67174757 | 67176747 | I | chr3 | 8715726 | 8717553 | I |
| chrX | 11997155 | 12008971 | I | chr3 | 8716460 | 8718255 | I |
| chr17 | 83274160 | 83278650 | I | chr7 | 143436364 | 143438519 | I |
| chr17 | 83275647 | 83277252 | I | chr7 | 143437354 | 143439098 | I |
| chr4 | 97751323 | 97753083 | I | chr16 | 17814505 | 17817397 | I |
| chr6 | 51168264 | 51170573 | I | chr16 | 17814589 | 17816465 | I |
| chr6 | 51168239 | 51170317 | I | chr7 | 125391861 | 125394445 | I |
| chr6 | 51167002 | 51170726 | I | chr5 | 135275110 | 135278091 | I |
| chr6 | 51168001 | 51169898 | I | chr3 | 131105716 | 131106723 | I |
| chr13 | 105293298 | 105294772 | I | chr9 | 107735018 | 107737194 | I |
| chr13 | 105293614 | 105295142 | I | chr15 | 102355071 | 102356422 | I |
| chr13 | 105292984 | 105294355 | I | chr14 | 21982814 | 21983918 | I |
| chr8 | 88453032 | 88455855 | I | chr5 | 33691540 | 33697642 | I |
| chr8 | 88452591 | 88455347 | I | chr5 | 33691776 | 33700575 | I |
| chr5 | 33689123 | 33690592 | I | chr5 | 33694991 | 33696328 | I |
| chr5 | 33689049 | 33690638 | I | chr5 | 33694603 | 33696196 | I |
| chr3 | 95971891 | 95974042 | I | chr3 | 87958396 | 87961173 | I |
| chr17 | 84095656 | 84098450 | I | chr3 | 87957503 | 87961307 | I |
| chr2 | 25166758 | 25170955 | I | chr3 | 87959492 | 87961061 | I |
| chr15 | 82962714 | 82964805 | I | chr3 | 87959467 | 87960467 | I |
| chr1 | 178275224 | 178277050 | I | chr18 | 35888285 | 35890978 | I |
| chr5 | 112395190 | 112396466 | I | chr17 | 15829804 | 15830804 | I |
| chr11 | 119052236 | 119053631 | I | chr1 | 4570998 | 4572517 | I |
| chrX | 11806186 | 11807576 | I | chr1 | 4571093 | 4572816 | I |
| chr3 | 130945717 | 130947077 | I | chr1 | 4570113 | 4574931 | I |
| chr5 | 135255234 | 135259451 | I | chr17 | 81669362 | 81671486 | I |
| chr1 | 165280446 | 165281448 | I | chr17 | 81669141 | 81671689 | I |
| chr14 | 25317278 | 25319610 | I | chr17 | 81668952 | 81669952 | I |
| chr14 | 78731405 | 78733811 | I | chr17 | 94886231 | 94887235 | II |
| chr7 | 98816167 | 98820265 | I | chr4 | 146049526 | 146051283 | II |
| chr9 | 67633796 | 67634796 | I | chr2 | 57628583 | 57630623 | II |
| chr9 | 32031389 | 32032988 | I | chr5 | 15673165 | 15674168 | II |
| chr11 | 119055661 | 119057010 | I | chr5 | 26008689 | 26009689 | II |
| chr11 | 119055004 | 119056811 | I | chr5 | 26069420 | 26070422 | II |
| chr9 | 72131256 | 72134380 | I | chr5 | 26079120 | 26080120 | II |
| chr14 | 65342719 | 65346277 | I | chr11 | 16514994 | 16516748 | II |
| chr4 | 44233749 | 44234751 | I | chr8 | 20029483 | 20033895 | II |
| chr4 | 8646714 | 8649686 | I | chr12 | 20195844 | 20197650 | II |
| chr4 | 97772025 | 97773025 | I | chr12 | 115203777 | 115204777 | II |
| chr9 | 63107452 | 63109561 | I | chr12 | 115503937 | 115505243 | II |
| chr5 | 149030680 | 149032636 | I | chr13 | 65484193 | 65486354 | II |
| chr4 | 97772648 | 97773677 | I | chr13 | 119965379 | 119966605 | II |
| chrX | 11663944 | 11666029 | I | chr13 | 65904835 | 65909173 | II |
| chr6 | 127329224 | 127331603 | I | chr14 | 5899141 | 5901257 | II |
| chr2 | 18763385 | 18768353 | I | chr14 | 5908742 | 5909747 | II |
| chr2 | 18764419 | 18769914 | I | chr14 | 7657613 | 7659864 | II |
| chr10 | 59969723 | 59971931 | I | chr14 | 19606481 | 19607481 | II |
| chr2 | 18765345 | 18767356 | I | chr16 | 3349141 | 3350143 | II |
| chr2 | 18766289 | 18768518 | I | chr14 | 106331485 | 106332494 | II |
| chr2 | 18765766 | 18768600 | I | chrX | 15261714 | 15262715 | II |
| chr5 | 142822274 | 142823275 | I | chr5 | 26067605 | 26069438 | II |
| chr2 | 30692932 | 30694859 | I | chr5 | 26074304 | 26076026 | II |
| chr13 | 28957513 | 28960252 | I | chr12 | 22029550 | 22030553 | II |
| chr2 | 135658304 | 135659305 | I | chr5 | 26077854 | 26080667 | II |
| chr15 | 85669502 | 85673130 | I | chr15 | 21656502 | 21657512 | II |
| chr15 | 85669731 | 85673988 | I | chr16 | 68986034 | 68987034 | II |
| chr15 | 85670455 | 85672787 | I | chr4 | 79045822 | 79046822 | II |
| chr14 | 5901693 | 5905693 | II | chr14 | 7243524 | 7245696 | II |
| chr13 | 120096133 | 120097338 | II | chr14 | 7242570 | 7246025 | II |
| chr14 | 5897793 | 5902102 | II | chr14 | 7244703 | 7245732 | II |
| chr14 | 6427043 | 6428092 | II | chr14 | 7244054 | 7245709 | II |
| chr14 | 6426053 | 6429024 | II | chr14 | 4018005 | 4020161 | II |
| chr14 | 7243750 | 7245703 | II | chr14 | 4017874 | 4019880 | II |
| chr12 | 115572035 | 115573698 | II | chr14 | 4018483 | 4019748 | II |
| chr12 | 22029643 | 22031118 | II | chr3 | 62159311 | 62160458 | II |
| chr12 | 115612696 | 115613696 | II | chr2 | 150500061 | 150502304 | II |
| chr12 | 115799148 | 115800516 | II | chr10 | 6206095 | 6207103 | II |
| chr13 | 65989825 | 65992049 | II | chr11 | 21990738 | 21991738 | II |
| chr13 | 120094527 | 120096465 | II | chr4 | 145450500 | 145451783 | II |
| chr17 | 53151566 | 53153518 | II | chr14 | 6426099 | 6428041 | II |
| chr14 | 7243755 | 7245988 | II | chr14 | 6426117 | 6428021 | II |
| chr14 | 6426127 | 6428037 | II | chr17 | 47107141 | 47108143 | II |
| chr14 | 6426240 | 6428063 | II | chr9 | 98051422 | 98052886 | II |
| chr14 | 6425769 | 6428060 | II | chr13 | 94705463 | 94706682 | II |
| chr14 | 7657995 | 7659598 | II | chr2 | 128255849 | 128256849 | II |
| chr14 | 7657615 | 7659347 | II | chr16 | 12930921 | 12931933 | II |
| chr14 | 7657355 | 7659259 | II | chr5 | 41754951 | 41755953 | II |
| chr14 | 7657583 | 7659631 | II | chr18 | 36256589 | 36257589 | II |
| chr14 | 7657348 | 7659601 | II | chr18 | 36256589 | 36257589 | II |
| chr14 | 7657326 | 7659580 | II | chr18 | 36256588 | 36257588 | II |
| chr12 | 115549429 | 115552846 | II | chr18 | 36256588 | 36257588 | II |
| chr12 | 114118469 | 114121030 | II | chr18 | 36256588 | 36257588 | II |
| chr12 | 115216325 | 115219276 | II | chr9 | 40869930 | 40871194 | II |
| chr12 | 115215315 | 115219471 | II | chr9 | 89882413 | 89883424 | II |
| chr12 | 115214978 | 115219471 | II | chr9 | 45021166 | 45022166 | II |
| chr10 | 7387867 | 7388950 | II | chr4 | 87604371 | 87605371 | II |
| chr12 | 115596851 | 115598622 | II | chr15 | 41276961 | 41277963 | II |
| chr3 | 19009182 | 19010182 | II | chr1 | 171084334 | 171086501 | II |
| chr3 | 19009182 | 19010182 | II | chr17 | 19713075 | 19715036 | II |
| chr5 | 44401339 | 44402341 | II | chr12 | 114803731 | 114805702 | II |
| chr7 | 95456206 | 95457220 | II | chr12 | 115683804 | 115685626 | II |
| chr12 | 115549048 | 115553855 | II | chr12 | 114649054 | 114650351 | II |
| chr12 | 115550069 | 115553839 | II | chr12 | 18134429 | 18137046 | II |
| chr12 | 115551376 | 115553785 | II | chr12 | 18134155 | 18137038 | II |
| chr12 | 115819733 | 115822119 | II | chr12 | 115571730 | 115575462 | II |
| chr12 | 115819387 | 115822572 | II | chr12 | 115571756 | 115574766 | II |
| chrX | 137099535 | 137101182 | II | chr12 | 115806468 | 115808428 | II |
| chr16 | 3113730 | 3117330 | II | chr17 | 69075431 | 69077643 | III |
| chr13 | 60442418 | 60443881 | II | chr17 | 69074712 | 69077151 | III |
| chr9 | 89879817 | 89880818 | II | chr17 | 69075840 | 69078416 | III |
| chr17 | 36064826 | 36066548 | II | chr17 | 69075299 | 69076299 | III |
| chr10 | 33665981 | 33666981 | II | chr17 | 69073557 | 69078906 | III |
| chr3 | 140328408 | 140329408 | II | chr1 | 133162990 | 133165300 | III |
| chr17 | 36077430 | 36078996 | II | chr1 | 133163210 | 133165260 | III |
| chr5 | 15043891 | 15045971 | II | chr1 | 133163219 | 133164978 | III |
| chr2 | 74777822 | 74778822 | II | chr1 | 133164064 | 133165086 | III |
| chr6 | 51179898 | 51180907 | II | chr1 | 133163739 | 133164776 | III |
| chr6 | 51179264 | 51181061 | II | chr5 | 67227607 | 67229825 | III |
| chr6 | 51179086 | 51180542 | II | chr5 | 67228152 | 67233398 | III |
| chr12 | 115816407 | 115819495 | II | chr5 | 67228791 | 67229796 | III |
| chr12 | 115816439 | 115819336 | II | chr5 | 67229975 | 67231977 | III |
| chr12 | 115816507 | 115819095 | II | chr5 | 67230593 | 67231893 | III |
| chr13 | 56208025 | 56209025 | II | chr5 | 67229455 | 67231413 | III |
| chr13 | 53462697 | 53463697 | II | chr5 | 67215123 | 67217151 | III |
| chr5 | 67216251 | 67217251 | III | chr9 | 87733834 | 87734921 | III |
| chr5 | 67215533 | 67216825 | III | chr12 | 58214614 | 58215649 | III |
| chr2 | 118765796 | 118767968 | III | chr7 | 89499950 | 89501604 | III |
| chr2 | 119315402 | 119318225 | III | chr11 | 90416630 | 90421094 | III |
| chr4 | 100041605 | 100043581 | III | chr3 | 95982166 | 95983466 | III |
| chr6 | 72793312 | 72795443 | III | chr7 | 19315692 | 19316692 | III |
| chr7 | 130108165 | 130109563 | III | chr9 | 40794327 | 40795380 | III |
| chr4 | 129749953 | 129752554 | III | chr9 | 23034035 | 23037130 | III |
| chr4 | 129749605 | 129751885 | III | chr7 | 100974484 | 100975492 | III |
| chr4 | 133899417 | 133900417 | III | chr9 | 43269475 | 43276623 | III |
| chr16 | 55758683 | 55759684 | III | chr7 | 73573436 | 73576292 | III |
| chr16 | 55758426 | 55759686 | III | chr6 | 145275443 | 145276526 | III |
| chr5 | 29467735 | 29469142 | III | chr4 | 141271966 | 141272967 | III |
| chr12 | 57549306 | 57551066 | III | chr15 | 80571637 | 80572719 | III |
| chr7 | 96080538 | 96081539 | III | chr10 | 13495836 | 13496838 | III |
| chr11 | 119058237 | 119060036 | III | chr10 | 13495836 | 13496836 | III |
| chr7 | 90301607 | 90302931 | III | chr2 | 18605754 | 18611371 | III |
| chr13 | 98587623 | 98591025 | III | chr2 | 18606286 | 18610713 | III |
| chr3 | 145632487 | 145633534 | III | chr2 | 18606259 | 18613057 | III |
| chr3 | 145632751 | 145633920 | III | chr2 | 18608816 | 18610148 | III |
| chr10 | 22842322 | 22844642 | III | chr2 | 18608172 | 18611014 | III |
| chr10 | 22842479 | 22844439 | III | chr2 | 18606908 | 18609947 | III |
| chr10 | 22843445 | 22844446 | III | chr2 | 18607710 | 18611146 | III |
| chr10 | 22843592 | 22845927 | III | chr2 | 18607664 | 18610476 | III |
| chr10 | 22842020 | 22845382 | III | chr2 | 18607437 | 18610004 | III |
| chr19 | 43598155 | 43601213 | III | chr2 | 18608129 | 18610043 | III |
| chr19 | 43598656 | 43602492 | III | chr2 | 18607293 | 18609343 | III |
| chr19 | 43599603 | 43601720 | III | chr8 | 19979180 | 19983264 | III |
| chr19 | 43598792 | 43600637 | III | chr2 | 22743134 | 22746826 | III |
| chr19 | 43598114 | 43604067 | III | chr2 | 22743895 | 22746096 | III |
| chr19 | 43605579 | 43607753 | III | chr2 | 22743883 | 22745123 | III |
| chr1 | 184725265 | 184726386 | III | chr9 | 66051153 | 66052153 | III |
| chr1 | 184723825 | 184726925 | III | chr9 | 66051153 | 66052153 | III |
| chr10 | 44526333 | 44527917 | III | chr19 | 41441393 | 41442406 | III |
| chr10 | 44526555 | 44528016 | III | chr9 | 77420519 | 77422155 | III |
| chr10 | 44527094 | 44528747 | III | chr4 | 135204900 | 135207356 | III |
| chr10 | 44526484 | 44529159 | III | chr2 | 69802529 | 69804442 | III |
| chr19 | 56776119 | 56777562 | III | chr11 | 53563369 | 53564370 | III |
| chr16 | 91533662 | 91534700 | III | chr2 | 118943999 | 118945552 | III |
| chr16 | 91534629 | 91535659 | III | chr2 | 160361189 | 160362189 | III |
| chr19 | 45694557 | 45695588 | III | chr2 | 160360890 | 160363523 | III |
| chr6 | 97182072 | 97183075 | III | chr4 | 8682982 | 8684639 | III |
| chr2 | 32087062 | 32088062 | III | chr2 | 170093867 | 170094869 | III |
| chr2 | 170094811 | 170095811 | III | chr12 | 112802724 | 112804486 | III |
| chr5 | 135088834 | 135092205 | III | chr6 | 38687895 | 38689397 | III |
| chr5 | 135088230 | 135091014 | III | chr17 | 80009273 | 80010485 | III |
| chr11 | 121323590 | 121324597 | III | chr13 | 56762876 | 56767905 | III |
| chr8 | 86603018 | 86604018 | III | chr5 | 52112084 | 52113272 | III |
| chr18 | 64332242 | 64334061 | III | chr5 | 52111812 | 52113413 | III |
| chr18 | 64330943 | 64333781 | III | chr7 | 31176417 | 31179189 | III |
| chr13 | 23401129 | 23403157 | III | chr13 | 23411472 | 23412656 | III |
| chr2 | 170458764 | 170460717 | III | chr2 | 90890721 | 90891721 | III |
| chr19 | 45675709 | 45677555 | III | chr15 | 75645469 | 75647949 | III |
| chr19 | 45675561 | 45677079 | III | chr4 | 55473604 | 55476408 | III |
| chr19 | 45675227 | 45676595 | III | chr15 | 83744804 | 83746629 | III |
| chr19 | 45675230 | 45678011 | III | chr17 | 45226125 | 45227658 | III |
| chr19 | 45675152 | 45678504 | III | chr17 | 45226126 | 45227128 | III |
| chr5 | 106755978 | 106757793 | III | chr12 | 112696261 | 112702132 | III |
| chr5 | 106756277 | 106759251 | III | chr12 | 112699334 | 112702951 | III |
| chr2 | 70471386 | 70472387 | III | chr2 | 173514861 | 173518450 | III |
| chr13 | 48705486 | 48707199 | III | chr1 | 167659310 | 167662614 | III |
| chr5 | 119005364 | 119011638 | III | chr11 | 85994989 | 85996785 | III |
| chr13 | 51593928 | 51595942 | III | chr11 | 104099174 | 104100549 | III |
| chr16 | 90519084 | 90520750 | III | chr4 | 136080499 | 136082469 | III |
| chr4 | 124178013 | 124179013 | III | chr4 | 136079899 | 136082107 | III |
| chr4 | 124176075 | 124179602 | III | chr4 | 136080025 | 136082848 | III |
| chr14 | 118472906 | 118478104 | III | chr11 | 119046992 | 119048844 | III |
| chr2 | 18688853 | 18690202 | III | chr5 | 119705024 | 119708218 | III |
| chr2 | 18688532 | 18690328 | III | chr5 | 119706314 | 119707355 | III |
| chr2 | 18688345 | 18690664 | III | chr6 | 70966210 | 70967722 | III |
| chr2 | 18688841 | 18693251 | III | chr6 | 71048023 | 71049714 | III |
| chr2 | 18687832 | 18691476 | III | chr11 | 95644445 | 95645445 | III |
| chr2 | 18688744 | 18691259 | III | chr5 | 37900945 | 37902280 | III |
| chr2 | 18688665 | 18691202 | III | chr17 | 7826561 | 7828386 | III |
| chr2 | 18688858 | 18691779 | III | chr2 | 118896776 | 118897776 | III |
| chr8 | 88451105 | 88453532 | III | chr1 | 191125447 | 191126893 | III |
| chr8 | 88450964 | 88452325 | III | chr1 | 191123781 | 191126778 | III |
| chr8 | 88449225 | 88451783 | III | chr19 | 3422180 | 3425537 | III |
| chr17 | 48093525 | 48098564 | III | chr7 | 99248388 | 99250619 | III |
| chr17 | 48094048 | 48098564 | III | chr7 | 99248304 | 99250291 | III |
| chr17 | 48094020 | 48102334 | III | chr7 | 99248368 | 99250951 | III |
| chr11 | 112788627 | 112789627 | III | chr14 | 25448419 | 25449999 | III |
| chr8 | 33516777 | 33518466 | III | chr14 | 25447843 | 25450078 | III |
| chr1 | 155207156 | 155208158 | III | chr1 | 166042655 | 166043919 | III |
| chr1 | 155207156 | 155208158 | III | chr4 | 136083235 | 136084242 | III |
| chr4 | 55476692 | 55478771 | III | chr4 | 136081171 | 136084808 | III |
| chr9 | 71399486 | 71401027 | III | chr13 | 98571362 | 98575045 | III |
| chr18 | 75359876 | 75363337 | III | chr13 | 98501311 | 98503619 | III |
| chr6 | 85408295 | 85411204 | III | chr13 | 98499310 | 98514030 | III |
| chr6 | 85407944 | 85411574 | III | chr13 | 98496248 | 98509799 | III |
| chr1 | 42416251 | 42417685 | III | chr3 | 107581462 | 107585399 | III |
| chr19 | 44880048 | 44881533 | III | chr3 | 107578723 | 107586104 | III |
| chr17 | 80846225 | 80848377 | III | chr3 | 107581258 | 107582556 | III |
| chr10 | 119066116 | 119067118 | III | chr3 | 107582095 | 107584154 | III |
| chr4 | 43719700 | 43720703 | III | chr3 | 107581819 | 107584308 | III |
| chr4 | 43719699 | 43720700 | III | chr3 | 107583134 | 107584134 | III |
| chr2 | 36008779 | 36013475 | III | chr12 | 33968574 | 33969574 | III |
| chr2 | 36007419 | 36013277 | III | chr1 | 14506052 | 14508276 | III |
| chr2 | 36010701 | 36013100 | III | chr5 | 37816333 | 37819452 | III |
| chr2 | 36010539 | 36013326 | III | chr5 | 37816304 | 37819206 | III |
| chr13 | 116710188 | 116711208 | III | chr5 | 37816899 | 37819017 | III |
| chr13 | 116313554 | 116314719 | III | chr5 | 37817592 | 37819012 | III |
| chr13 | 116704641 | 116712964 | III | chr5 | 37816636 | 37817934 | III |
| chr13 | 116705377 | 116707817 | III | chr5 | 37816395 | 37818090 | III |
| chr4 | 148769560 | 148771093 | III | chr5 | 37815696 | 37818353 | III |
| chr1 | 186833017 | 186835390 | III | chr5 | 37828309 | 37832387 | III |
| chr5 | 53257472 | 53258841 | III | chr5 | 37813988 | 37819750 | III |
| chr4 | 97744950 | 97747223 | III | chr5 | 37815387 | 37817294 | III |
| chr4 | 97745047 | 97746896 | III | chr5 | 37827841 | 37828932 | III |
| chr4 | 97744863 | 97748883 | III | chr5 | 37828045 | 37829595 | III |
| chr1 | 86017403 | 86018760 | III | chr1 | 78213776 | 78222076 | III |
| chr5 | 137189298 | 137191045 | III | chr1 | 78215490 | 78216988 | III |
| chr18 | 36319080 | 36321951 | III | chr1 | 78215860 | 78218245 | III |
| chr2 | 122466371 | 122468267 | III | chr1 | 78215590 | 78218083 | III |
| chr2 | 118971221 | 118979015 | III | chr1 | 78215923 | 78217956 | III |
| chr2 | 118973301 | 118977667 | III | chr1 | 78215467 | 78219179 | III |
| chr13 | 72636344 | 72640748 | III | chr1 | 78216396 | 78217662 | III |
| chr13 | 72634690 | 72641952 | III | chr1 | 78216603 | 78218404 | III |
| chr13 | 72638802 | 72640688 | III | chr1 | 78217067 | 78218508 | III |
| chr13 | 72624226 | 72625689 | III | chr9 | 120732884 | 120734634 | III |
| chr12 | 56301716 | 56302717 | III | chr5 | 111530109 | 111532710 | III |
| chr12 | 56301850 | 56303129 | III | chr13 | 98504198 | 98505198 | III |
| chr12 | 56299808 | 56302221 | III | chr5 | 135276803 | 135278357 | III |
| chr2 | 148032921 | 148035465 | III | chr8 | 88381790 | 88383927 | III |
| chr2 | 148033162 | 148035434 | III | chr11 | 85916280 | 85919140 | III |
| chr2 | 148030866 | 148032858 | III | chr13 | 53461075 | 53463916 | III |
| chr2 | 148030148 | 148032446 | III | chr13 | 53462077 | 53463077 | III |
| chr2 | 148029489 | 148031297 | III | chr13 | 53465649 | 53466868 | III |
| chr2 | 148028519 | 148030196 | III | chr13 | 53465550 | 53466656 | III |
| chr13 | 53464224 | 53465880 | III | chr6 | 53792945 | 53797308 | III |
| chr13 | 53463890 | 53464890 | III | chr11 | 4749383 | 4751747 | III |
| chr13 | 53475910 | 53477131 | III | chr18 | 76376933 | 76380675 | III |
| chr12 | 73050649 | 73052820 | III | chr12 | 80738693 | 80740532 | III |
| chr12 | 73050826 | 73052452 | III | chr4 | 150032284 | 150033576 | III |
| chr12 | 73052267 | 73054385 | III | chr4 | 150031932 | 150033845 | III |
| chr12 | 33964324 | 33969057 | III | chr19 | 45544624 | 45546161 | III |
| chr12 | 33965015 | 33967715 | III | chr1 | 190664859 | 190667018 | III |
| chr12 | 33967042 | 33968042 | III | chr9 | 27213192 | 27214192 | III |
| chr12 | 33961253 | 33964144 | III | chr4 | 150029395 | 150031740 | III |
| chr13 | 48668186 | 48670023 | III | chr19 | 45226634 | 45227646 | III |
| chr13 | 48668558 | 48669852 | III | chr14 | 68055032 | 68056685 | III |
| chr13 | 48668786 | 48671255 | III | chr19 | 44733799 | 44736426 | III |
| chr13 | 48658636 | 48660378 | III | chr14 | 104472320 | 104474377 | III |
| chr13 | 55962455 | 55965679 | III | chr19 | 44683024 | 44684762 | III |
| chr13 | 55961237 | 55963568 | III | chr19 | 44682764 | 44684683 | III |
| chr15 | 103111247 | 103117492 | III | chr19 | 44681637 | 44684408 | III |
| chr8 | 25354534 | 25355534 | III | chr5 | 101657081 | 101658081 | III |
| chr13 | 98385987 | 98394622 | III | chr5 | 101656870 | 101657872 | III |
| chr5 | 136892595 | 136896887 | III | chr2 | 169360962 | 169361962 | III |
| chr17 | 85691663 | 85694473 | III | chr13 | 40709747 | 40712092 | III |
| chr17 | 85691037 | 85693009 | III | chr11 | 33193226 | 33195440 | III |
| chr17 | 85678542 | 85680820 | III | chr9 | 40855287 | 40860529 | III |
| chr9 | 32547586 | 32548682 | III | chr13 | 94869647 | 94871501 | III |
| chr6 | 51174665 | 51175955 | III | chr12 | 55537685 | 55539538 | III |
| chrX | 145515025 | 145516029 | III | chr6 | 52318685 | 52320551 | III |
| chr5 | 119659338 | 119664156 | III | chr5 | 139567643 | 139592672 | III |
| chr5 | 119662199 | 119664967 | III | chr2 | 9889752 | 9891194 | III |
| chr11 | 85925483 | 85926881 | III | chr5 | 139667338 | 139670435 | III |
| chr11 | 85925405 | 85926662 | III | chr5 | 139666437 | 139670596 | III |
| chr11 | 85924230 | 85926299 | III | chr5 | 139666542 | 139668659 | III |
| chr6 | 52253542 | 52254542 | III | chr5 | 139667014 | 139668787 | III |
| chr6 | 52252822 | 52254497 | III | chr5 | 139668419 | 139669419 | III |
| chr3 | 99240932 | 99242642 | III | chr2 | 9895187 | 9896596 | III |
| chr3 | 99241021 | 99242942 | III | chr2 | 9894908 | 9896254 | III |
| chr3 | 99240512 | 99243204 | III | chr2 | 9894254 | 9897325 | III |
| chr3 | 99239822 | 99241129 | III | chr19 | 45227689 | 45230361 | III |
| chr5 | 33538139 | 33543608 | III | chr19 | 45225362 | 45227452 | III |
| chr19 | 45220503 | 45224874 | III | chr11 | 33199003 | 33200666 | III |
| chr4 | 153188635 | 153190522 | III | chr19 | 45222532 | 45225377 | III |
| chr11 | 76666335 | 76667351 | III | chr19 | 45222096 | 45225300 | III |
| chr6 | 126700503 | 126702595 | III | chr19 | 45222431 | 45224756 | III |
| chr6 | 126700339 | 126703074 | III | chr18 | 34678442 | 34680611 | III |
| chr7 | 37438482 | 37439482 | III | chr13 | 94708311 | 94712195 | III |
| chr1 | 89926255 | 89927255 | III | chr13 | 94595178 | 94597176 | III |
| chr1 | 187604780 | 187605781 | III | chr13 | 94594348 | 94596783 | III |
| chr1 | 187604186 | 187605958 | III | chr13 | 94594207 | 94598138 | III |
| chr12 | 110372636 | 110376422 | III | chr4 | 44424917 | 44426967 | III |
| chr10 | 80468799 | 80473214 | III | chr4 | 44424094 | 44426480 | III |
| chr8 | 87036165 | 87039612 | III | chr15 | 99094006 | 99095389 | III |
| chr10 | 63088906 | 63092287 | III | chr5 | 139554124 | 139556568 | III |
| chr12 | 104637166 | 104639159 | III | chr11 | 84561568 | 84565690 | III |
| chr4 | 115961877 | 115965228 | III | chr11 | 84562710 | 84564016 | III |
| chr15 | 98795702 | 98797385 | III | chr4 | 143324627 | 143327535 | III |
| chr15 | 75644220 | 75646909 | III | chr2 | 165562145 | 165564071 | III |
| chr4 | 136904302 | 136906998 | III | chr7 | 143444017 | 143445032 | III |
| chr16 | 78234118 | 78236693 | III | chr15 | 98798064 | 98799064 | III |
| chr15 | 91077908 | 91078908 | III | chr15 | 98797274 | 98798950 | III |
| chr19 | 28974784 | 28976012 | III | chr1 | 78201423 | 78203111 | III |
| chr17 | 83544493 | 83545956 | III | chr1 | 78201409 | 78203694 | III |
| chr5 | 33460960 | 33467861 | III | chr1 | 78199873 | 78202500 | III |
| chr4 | 123991289 | 123992956 | III | chr1 | 78201133 | 78202630 | III |
| chr9 | 69755231 | 69756231 | III | chr1 | 78200758 | 78201995 | III |
| chr14 | 72531301 | 72533440 | III | chr1 | 78200421 | 78201421 | III |
| chr16 | 91232106 | 91233106 | III | chr9 | 99868717 | 99871465 | III |
| chr16 | 91300035 | 91303080 | III | chr9 | 99869634 | 99871581 | III |
| chr16 | 91299578 | 91303844 | III | chr9 | 99869941 | 99870941 | III |
| chr9 | 69668108 | 69672316 | III | chr9 | 99870022 | 99871099 | III |
| chr12 | 74283501 | 74285184 | III | chr9 | 99869589 | 99871048 | III |
| chr18 | 38023372 | 38027336 | III | chr8 | 78427073 | 78428073 | III |
| chr18 | 42428469 | 42431581 | III | chr9 | 99868588 | 99869829 | III |
| chr1 | 190688968 | 190692523 | III | chr9 | 99868662 | 99869662 | III |
| chr4 | 13563673 | 13567009 | III | chr9 | 99868083 | 99869639 | III |
| chr8 | 126138140 | 126141291 | III | chr9 | 99867807 | 99869724 | III |
| chr8 | 126137950 | 126139931 | III | chr9 | 99869969 | 99872234 | III |
| chr8 | 126138700 | 126140586 | III | chr9 | 99869321 | 99872631 | III |
| chr8 | 126138616 | 126140162 | III | chr9 | 99868089 | 99873492 | III |
| chr15 | 103161945 | 103190307 | III | chr9 | 99871132 | 99872991 | III |
| chr9 | 56824104 | 56826786 | III | chr9 | 99871123 | 99872855 | III |
| chr11 | 84598648 | 84600359 | III | chr9 | 99871184 | 99872582 | III |
| chr11 | 84599364 | 84600364 | III | chr9 | 99870956 | 99872410 | III |
| chr9 | 107376825 | 107384066 | III | chr1 | 120612058 | 120618044 | III |
| chr4 | 120358718 | 120362283 | III | chr5 | 28156013 | 28160871 | III |
| chr9 | 40858430 | 40859571 | III | chr1 | 120610828 | 120611828 | III |
| chr9 | 40858004 | 40859005 | III | chr5 | 28115577 | 28129068 | III |
| chr13 | 94710006 | 94711134 | III | chr5 | 28116704 | 28125730 | III |
| chr5 | 28120328 | 28125973 | III | chrX | 57920123 | 57922642 | III |
| chr5 | 28124052 | 28125982 | III | chr9 | 37454795 | 37457530 | III |
| chr5 | 28115858 | 28120181 | III | chrX | 58018871 | 58019871 | III |
| chr5 | 28117135 | 28118806 | III | chr7 | 44425833 | 44427763 | III |
| chr19 | 25605279 | 25606641 | III | chr9 | 46442366 | 46443883 | III |
| chr19 | 25606127 | 25607809 | III | chr10 | 80191216 | 80192972 | III |
| chr4 | 109974305 | 109975308 | III | chr6 | 91285452 | 91286452 | III |
| chr4 | 109974019 | 109975233 | III | chr2 | 180488275 | 180489872 | III |
| chr2 | 172727268 | 172730326 | III | chr2 | 180487384 | 180489838 | III |
| chr2 | 172727141 | 172729067 | III | chr2 | 73278805 | 73280027 | III |
| chr4 | 109984546 | 109985578 | III | chr2 | 73277675 | 73279245 | III |
| chr19 | 59465975 | 59472829 | III | chr2 | 73279307 | 73280783 | III |
| chr5 | 106463167 | 106465272 | III | chr11 | 98316530 | 98318564 | III |
| chr3 | 127641761 | 127642779 | III | chr4 | 125476795 | 125480047 | III |
| chrX | 60889567 | 60890567 | III | chr11 | 98320188 | 98322141 | III |
| chr3 | 127638523 | 127641543 | III | chr11 | 98319840 | 98322763 | III |
| chr3 | 127638243 | 127639998 | III | chr11 | 98319437 | 98321176 | III |
| chr3 | 127625174 | 127629722 | III | chr11 | 119089422 | 119090422 | III |
| chr17 | 85484439 | 85487901 | III | chr11 | 119089603 | 119090604 | III |
| chr17 | 85484394 | 85487727 | III | chr16 | 34744726 | 34746556 | III |
| chr17 | 85485755 | 85487562 | III | chr3 | 149087246 | 149089223 | III |
| chr17 | 85483501 | 85490765 | III | chr3 | 149087870 | 149090193 | III |
| chr17 | 85463529 | 85467313 | III | chr3 | 9345650 | 9347553 | III |
| chr17 | 85462971 | 85467157 | III | chr11 | 101936183 | 101938332 | III |
| chr17 | 85464499 | 85466687 | III | chr14 | 64045546 | 64046939 | III |
| chr11 | 21992116 | 21994349 | III | chr15 | 28049576 | 28052559 | III |
| chr11 | 21993210 | 21994681 | III | chr9 | 48644682 | 48651288 | III |
| chr11 | 21992683 | 21993702 | III | chr11 | 119089125 | 119090911 | III |
| chr5 | 120321067 | 120322661 | III | chr11 | 119089110 | 119091724 | III |
| chr5 | 120321462 | 120323265 | III | chr11 | 119090070 | 119091070 | III |
| chr12 | 118842346 | 118843436 | III | chr13 | 37777638 | 37779454 | III |
| chr13 | 56257820 | 56259818 | III | chr13 | 37777359 | 37779665 | III |
| chr13 | 56257890 | 56259725 | III | chr13 | 37778688 | 37780011 | III |
| chr13 | 56257103 | 56259226 | III | chr18 | 11238665 | 11241362 | III |
| chr13 | 56255905 | 56260724 | III | chr18 | 11239798 | 11240979 | III |
| chr2 | 71542071 | 71543253 | III | chr5 | 136619811 | 136624345 | III |
| chr2 | 71541715 | 71542903 | III | chr4 | 123228438 | 123229560 | III |
| chr2 | 71540518 | 71543090 | III | chr5 | 74904864 | 74905864 | III |
| chr2 | 71540701 | 71542445 | III | chr1 | 184770605 | 184772410 | III |
| chr7 | 25180513 | 25181623 | III | chr4 | 130162865 | 130164371 | III |
| chr4 | 148172058 | 148174458 | III | chr3 | 149085360 | 149086360 | III |
| chr5 | 120301043 | 120302405 | III | chr16 | 38720520 | 38726204 | III |
| chr5 | 120298285 | 120305241 | III | chr5 | 147750554 | 147753347 | III |
| chr5 | 149020174 | 149021900 | III | chr12 | 109398762 | 109399819 | III |
| chr6 | 114967349 | 114968369 | III | chr7 | 143464459 | 143466013 | III |
| chr11 | 85844745 | 85845747 | III | chr4 | 106827285 | 106828599 | III |
| chr3 | 51357757 | 51359070 | III | chr17 | 3314101 | 3315102 | III |
| chr2 | 168049539 | 168051255 | III | chr13 | 48693502 | 48695608 | III |
| chr12 | 105013229 | 105014465 | III | chr13 | 48693140 | 48694468 | III |
| chr18 | 66474925 | 66476976 | III | chr10 | 89344391 | 89345571 | III |
| chr2 | 18587804 | 18589166 | III | chr15 | 85176787 | 85178747 | III |
| chr2 | 119318128 | 119319402 | III | chr17 | 26845073 | 26846085 | III |
| chr6 | 144779898 | 144781062 | III | chr10 | 80462344 | 80464877 | III |
| chr2 | 118749369 | 118752329 | III | chr11 | 52743091 | 52747765 | III |
| chr17 | 10370847 | 10371988 | III | chr1 | 171551940 | 171553649 | III |
| chr4 | 115052675 | 115053680 | III | chr17 | 26849170 | 26851064 | III |
| chr4 | 115051222 | 115053706 | III | chr17 | 26848888 | 26851172 | III |
| chr9 | 64059573 | 64061275 | III | chr14 | 63251599 | 63252599 | III |
| chr9 | 64059101 | 64062459 | III | chr14 | 63250188 | 63253351 | III |
| chr19 | 36347541 | 36350072 | III | chr14 | 63252986 | 63254087 | III |
| chr19 | 36347367 | 36349824 | III | chr14 | 63268801 | 63270764 | III |
| chr19 | 36347449 | 36350657 | III | chr14 | 63266359 | 63268515 | III |
| chr19 | 36347973 | 36349716 | III | chr14 | 63254072 | 63256124 | III |
| chr19 | 36347956 | 36348956 | III | chr5 | 119802440 | 119804487 | III |
| chr8 | 87973840 | 87978089 | III | chr5 | 119802056 | 119803633 | III |
| chr6 | 72184317 | 72192446 | III | chr5 | 119803183 | 119805099 | III |
| chr7 | 98870472 | 98873441 | III | chr5 | 119803666 | 119805264 | III |
| chr9 | 32548285 | 32550011 | III | chr5 | 119800119 | 119802175 | III |
| chr4 | 114688138 | 114689329 | III | chr12 | 78911053 | 78913373 | III |
| chr9 | 32544661 | 32546669 | III | chr12 | 78910848 | 78913741 | III |
| chr9 | 32544195 | 32545246 | III | chr3 | 89131790 | 89133464 | III |
| chr6 | 51173757 | 51178201 | III | chr4 | 115094637 | 115096600 | III |
| chr6 | 51174045 | 51175584 | III | chr4 | 115094154 | 115098468 | III |
| chr9 | 32408005 | 32410281 | III | chr15 | 80257715 | 80260138 | III |
| chr9 | 32407480 | 32410297 | III | chrX | 11997824 | 11999913 | III |
| chr9 | 32406886 | 32411018 | III | chr2 | 36196734 | 36207344 | III |
| chr3 | 30513449 | 30514449 | III | chr17 | 33977675 | 33979787 | III |
| chr3 | 30513296 | 30514298 | III | chr17 | 33977045 | 33979671 | III |
| chr3 | 30513711 | 30516989 | III | chr17 | 33976936 | 33979287 | III |
| chr3 | 30513469 | 30515848 | III | chr17 | 33976936 | 33979287 | III |
| chr3 | 30514374 | 30516471 | III | chr17 | 33978039 | 33979422 | III |
| chr11 | 94871683 | 94873187 | III | chr17 | 33977887 | 33979244 | III |
| chr7 | 83744860 | 83745886 | III | chr17 | 33977453 | 33979419 | III |
| chr5 | 74943855 | 74945787 | III | chr17 | 33977822 | 33978822 | III |
| chr5 | 74940651 | 74944935 | III | chr17 | 33976391 | 33979443 | III |
| chr19 | 45079854 | 45082337 | III | chr17 | 33976397 | 33979423 | III |
| chr17 | 33976210 | 33979422 | III | chr11 | 96276958 | 96278147 | III |
| chr17 | 33975408 | 33979423 | III | chr15 | 102999533 | 103000533 | III |
| chr12 | 57538192 | 57540253 | III | chr15 | 102999019 | 103000954 | III |
| chr12 | 57534744 | 57539830 | III | chr15 | 103000521 | 103001730 | III |
| chr12 | 57533620 | 57537296 | III | chr11 | 96404538 | 96406957 | III |
| chr12 | 57534398 | 57538070 | III | chr2 | 169995064 | 169996411 | III |
| chr2 | 148038574 | 148040834 | III | chr11 | 96407027 | 96408823 | III |
| chr2 | 148039148 | 148041208 | III | chr11 | 96406930 | 96408535 | III |
| chr2 | 148038576 | 148041079 | III | chr11 | 96406508 | 96408850 | III |
| chr2 | 148039111 | 148040111 | III | chr11 | 96407835 | 96409589 | III |
| chr2 | 148037571 | 148040933 | III | chr11 | 96405565 | 96408835 | III |
| chr2 | 148037402 | 148040369 | III | chr11 | 96406972 | 96409884 | III |
| chr13 | 98365161 | 98366343 | III | chr11 | 96405730 | 96409095 | III |
| chr11 | 95595912 | 95597679 | III | chr11 | 96405951 | 96407859 | III |
| chr19 | 44723555 | 44724890 | III | chr17 | 83997149 | 83999110 | III |
| chr19 | 44722980 | 44724585 | III | chr17 | 83997149 | 83999152 | III |
| chr15 | 103182459 | 103183459 | III | chr11 | 96310871 | 96313241 | III |
| chr15 | 103181298 | 103183369 | III | chr11 | 96311273 | 96312276 | III |
| chr15 | 103182197 | 103184166 | III | chr11 | 96310752 | 96311752 | III |
| chr13 | 94607476 | 94608970 | III | chr11 | 18875381 | 18876789 | III |
| chr17 | 23692979 | 23701905 | III | chr11 | 18874868 | 18876796 | III |
| chr5 | 35312771 | 35315772 | III | chr13 | 107893450 | 107896273 | IV |
| chr17 | 48545386 | 48546426 | III | chr10 | 29970956 | 29971974 | IV |
| chr19 | 44638867 | 44641833 | III | chr4 | 56190502 | 56191502 | IV |
| chr19 | 44638599 | 44641005 | III | chr11 | 54139754 | 54140759 | IV |
| chr19 | 44639861 | 44641465 | III | chr3 | 8245490 | 8246514 | IV |
| chr19 | 44667929 | 44675418 | III | chr5 | 51358374 | 51359375 | IV |
| chr15 | 103171325 | 103172697 | III | chr13 | 119420680 | 119423683 | IV |
| chr15 | 103067464 | 103069429 | III | chr17 | 84505825 | 84507152 | IV |
| chr15 | 103177179 | 103178601 | III | chr15 | 102958068 | 102959068 | IV |
| chr15 | 103177291 | 103178916 | III | chr4 | 3232434 | 3233435 | IV |
| chr15 | 102985480 | 102987660 | III | chr16 | 94081408 | 94082408 | IV |
| chr15 | 103069218 | 103071079 | III | chr16 | 94077521 | 94081925 | IV |
| chr15 | 103068535 | 103069728 | III | chr16 | 94078427 | 94079429 | IV |
| chr15 | 103039542 | 103041595 | III | chr15 | 28067971 | 28072409 | IV |
| chr19 | 44750496 | 44751684 | III | chr13 | 119615604 | 119618288 | IV |
| chr19 | 44749012 | 44751812 | III | chr17 | 87969609 | 87970609 | IV |
| chr19 | 44740060 | 44743705 | III | chr17 | 87969608 | 87970608 | IV |
| chr19 | 44739325 | 44743791 | III | chr17 | 87968646 | 87970611 | IV |
| chr15 | 103001696 | 103003826 | III | chr3 | 99239053 | 99240055 | IV |
| chr15 | 102995787 | 102998485 | III | chr6 | 51176536 | 51177536 | IV |
| chr15 | 103004760 | 103005819 | III | chr6 | 51175758 | 51178873 | IV |
| chr11 | 96276654 | 96278470 | III | chr6 | 51176052 | 51178308 | IV |
| chr6 | 51180630 | 51181824 | IV | chr1 | 42430078 | 42432454 | IV |
| chr6 | 51180052 | 51181506 | IV | chr4 | 81479791 | 81480907 | IV |
| chr2 | 118973413 | 118975340 | IV | chr19 | 44648839 | 44651554 | IV |
| chr11 | 76667819 | 76668930 | IV | chr5 | 101364327 | 101367380 | IV |
| chr2 | 163947445 | 163949032 | IV | chr19 | 44638621 | 44639621 | IV |
| chr12 | 118456679 | 118458243 | IV | chr19 | 44675495 | 44678258 | IV |
| chr12 | 112707841 | 112709694 | IV | chr18 | 42416275 | 42419852 | IV |
| chr2 | 18783027 | 18785869 | IV | chr4 | 115974572 | 115975572 | IV |
| chr19 | 45162513 | 45164721 | IV | chr9 | 89814247 | 89816135 | IV |
| chr9 | 98963755 | 98965321 | IV | chr19 | 45239799 | 45241804 | IV |
| chr5 | 35400588 | 35402264 | IV | chr18 | 76846836 | 76849201 | IV |
| chr5 | 35400833 | 35402565 | IV | chr5 | 29484513 | 29486442 | IV |
| chr5 | 35399036 | 35402902 | IV | chr5 | 29484422 | 29486081 | IV |
| chr17 | 43001838 | 43002849 | IV | chr5 | 29484529 | 29487804 | IV |
| chr2 | 172544955 | 172545955 | IV | chr17 | 8428819 | 8431756 | IV |
| chr2 | 127265460 | 127266462 | IV | chr17 | 8428619 | 8430961 | IV |
| chr17 | 36211555 | 36213792 | IV | chr17 | 8429819 | 8431890 | IV |
| chr14 | 103351086 | 103354015 | IV | chr17 | 8428563 | 8429897 | IV |
| chr3 | 88019292 | 88021151 | IV | chr16 | 17221351 | 17223379 | IV |
| chr13 | 110743071 | 110745351 | IV | chr16 | 17222263 | 17223387 | IV |
| chr6 | 83081373 | 83082510 | IV | chr11 | 90938052 | 90939053 | IV |
| chr10 | 121330616 | 121332970 | IV | chr3 | 107462434 | 107463434 | IV |
| chr8 | 45385940 | 45389441 | IV | chr19 | 24895654 | 24896654 | IV |
| chr13 | 21994409 | 21998339 | IV | chr5 | 29469485 | 29470488 | IV |
| chr19 | 45163746 | 45166441 | IV | chr9 | 98971193 | 98972779 | IV |
| chr13 | 25048853 | 25051910 | IV | chr10 | 57446708 | 57448237 | IV |
| chr5 | 75078443 | 75080442 | IV | chr16 | 98022153 | 98024152 | IV |
| chr13 | 47572496 | 47573496 | IV | chr11 | 104196237 | 104199150 | IV |
| chr2 | 153021477 | 153025108 | IV | chr19 | 36350930 | 36352426 | IV |
| chr19 | 43426578 | 43429456 | IV | chr7 | 3204885 | 3206803 | IV |
| chr4 | 118546652 | 118550133 | IV | chr14 | 71073168 | 71074171 | IV |
| chr4 | 118546745 | 118549646 | IV | chr12 | 84719867 | 84720867 | IV |
| chr4 | 118546806 | 118549380 | IV | chr10 | 57447707 | 57448812 | IV |
| chr5 | 101539660 | 101541522 | IV | chr10 | 57447361 | 57448831 | IV |
| chr1 | 13083578 | 13086039 | IV | chr5 | 52109699 | 52110699 | IV |
| chr1 | 78206038 | 78207038 | IV | chr18 | 42403474 | 42404474 | IV |
| chr10 | 91807157 | 91808158 | IV | chr6 | 52153370 | 52154370 | IV |
| chr5 | 101713487 | 101714491 | IV | chr10 | 7233703 | 7234706 | IV |
| chr1 | 172572785 | 172577225 | IV | chr9 | 3257870 | 3259867 | IV |
| chr17 | 34074165 | 34076394 | IV | chr9 | 3257875 | 3259846 | IV |
| chr4 | 115899235 | 115900274 | IV | chr18 | 68691133 | 68692187 | IV |
| chr4 | 124268490 | 124271976 | IV | chr11 | 109010840 | 109011976 | IV |
| chr18 | 54698300 | 54699301 | IV | chr11 | 109010683 | 109011918 | IV |
| chr3 | 5859498 | 5861747 | IV | chr15 | 80443236 | 80444854 | IV |
| chr11 | 109010688 | 109012659 | IV | chr19 | 24897052 | 24898583 | IV |
| chr11 | 109010690 | 109012661 | IV | chr19 | 24896882 | 24897882 | IV |
| chr11 | 109011098 | 109012665 | IV | chr19 | 24895192 | 24898148 | IV |
| chr11 | 109011725 | 109013067 | IV | chr9 | 122476537 | 122477537 | IV |
| chr11 | 109011938 | 109012950 | IV | chr8 | 13151648 | 13152649 | IV |
| chr16 | 17817167 | 17818184 | IV | chr19 | 45241504 | 45245306 | IV |
| chr11 | 22707470 | 22708470 | IV | chr19 | 45241141 | 45245330 | IV |
| chr10 | 117593524 | 117596272 | IV | chr19 | 45242424 | 45244421 | IV |
| chr13 | 48671023 | 48672025 | IV | chr19 | 45241251 | 45242636 | IV |
| chr10 | 80490384 | 80492238 | IV | chr17 | 36205255 | 36207536 | IV |
| chr8 | 109968062 | 109969540 | IV | chr17 | 36205046 | 36207192 | IV |
| chr2 | 118893549 | 118894549 | IV | chr17 | 36204011 | 36206851 | IV |
| chr3 | 132345856 | 132346856 | IV | chr17 | 36205175 | 36206805 | IV |
| chr1 | 180924093 | 180925856 | IV | chr17 | 36205202 | 36206380 | IV |
| chr6 | 23261237 | 23262238 | IV | chr17 | 36204759 | 36208616 | IV |
| chr1 | 184720742 | 184721742 | IV | chr17 | 36204615 | 36208915 | IV |
| chr4 | 52542876 | 52543876 | IV | chr17 | 36204622 | 36208715 | IV |
| chr18 | 64334567 | 64336943 | IV | chr17 | 36205002 | 36209413 | IV |
| chr4 | 120651301 | 120652303 | IV | chr2 | 116560100 | 116562225 | IV |
| chr7 | 49752831 | 49755089 | IV | chr2 | 116560267 | 116561975 | IV |
| chr4 | 9874061 | 9875506 | IV | chr2 | 116560381 | 116562407 | IV |
| chr19 | 43601701 | 43603298 | IV | chr2 | 116560675 | 116561964 | IV |
| chr19 | 43600607 | 43601934 | IV | chr2 | 116560721 | 116561853 | IV |
| chr17 | 35575748 | 35578315 | IV | chr2 | 116560439 | 116562020 | IV |
| chr2 | 125813662 | 125815476 | IV | chr9 | 89880250 | 89882336 | IV |
| chr19 | 45144186 | 45145889 | IV | chr9 | 89880504 | 89882505 | IV |
| chr11 | 105164752 | 105166720 | IV | chr9 | 89880289 | 89881936 | IV |
| chr19 | 45140543 | 45147706 | IV | chr9 | 89881434 | 89882434 | IV |
| chr19 | 45140879 | 45145980 | IV | chr9 | 89881229 | 89882820 | IV |
| chr19 | 25625335 | 25627544 | IV | chr9 | 89879785 | 89881765 | IV |
| chr5 | 35399214 | 35400794 | IV | chr2 | 116549814 | 116551520 | IV |
| chr5 | 35398786 | 35400075 | IV | chr2 | 116549503 | 116551425 | IV |
| chr10 | 80487181 | 80488871 | IV | chr2 | 116549680 | 116551426 | IV |
| chr4 | 46348173 | 46349973 | IV | chr2 | 116549677 | 116550726 | IV |
| chr19 | 25666213 | 25667663 | IV | chr2 | 116550679 | 116552170 | IV |
| chr19 | 25665324 | 25667455 | IV | chr18 | 68358940 | 68359941 | IV |
| chr4 | 139858467 | 139859467 | IV | chr18 | 68358940 | 68359942 | IV |
| chr2 | 172545935 | 172546944 | IV | chr3 | 96334079 | 96339352 | IV |
| chr9 | 98929287 | 98933232 | IV | chr1 | 171546806 | 171549711 | IV |
| chr9 | 98928740 | 98932196 | IV | chr3 | 107342047 | 107343921 | IV |
| chr4 | 139849405 | 139855543 | IV | chr2 | 116553882 | 116555473 | IV |
| chr17 | 56149894 | 56151049 | IV | chr2 | 116553217 | 116554738 | IV |
| chr2 | 116552300 | 116554900 | IV | chr2 | 116556424 | 116557424 | IV |
| chr2 | 116551421 | 116554893 | IV | chr2 | 150525083 | 150526089 | IV |
| chr9 | 89879417 | 89881648 | IV | chr2 | 150525674 | 150527121 | IV |
| chr9 | 89878875 | 89881787 | IV | chr1 | 171084297 | 171089363 | IV |
| chr17 | 36214657 | 36216926 | IV | chr17 | 50419150 | 50421138 | IV |
| chr17 | 36214594 | 36217104 | IV | chr2 | 73012734 | 73013881 | IV |
| chr17 | 36214591 | 36217326 | IV | chr12 | 56680292 | 56682335 | IV |
| chr17 | 36213829 | 36217746 | IV | chr12 | 56679462 | 56682225 | IV |
| chr4 | 118778404 | 118779584 | IV | chr12 | 56678807 | 56683354 | IV |
| chr15 | 60141750 | 60142750 | IV | chr14 | 56864219 | 56865985 | IV |
| chr19 | 38088602 | 38089792 | IV | chr19 | 37876258 | 37888520 | IV |
| chr14 | 108158281 | 108159284 | IV | chr14 | 46393189 | 46400679 | IV |
| chr2 | 116548030 | 116549056 | IV | chr4 | 116839680 | 116841658 | IV |
| chr5 | 101539541 | 101540623 | IV | chr12 | 85419951 | 85420951 | IV |
| chr4 | 109988404 | 109989404 | IV | chr1 | 4688007 | 4690164 | IV |
| chr4 | 109988403 | 109989404 | IV | chr1 | 4687955 | 4690187 | IV |
| chr2 | 116557072 | 116558141 | IV | chr2 | 71550527 | 71551527 | IV |
| chr2 | 116552488 | 116553572 | IV | chr2 | 71550949 | 71551954 | IV |
| chr6 | 85525161 | 85526718 | IV | chr13 | 23393640 | 23397379 | IV |
| chr2 | 116557719 | 116559928 | IV | chr13 | 23395059 | 23398301 | IV |
| chr2 | 116558106 | 116559466 | IV | chr18 | 38386503 | 38387503 | IV |
| chr2 | 116556760 | 116559836 | IV | chr9 | 30931443 | 30933940 | IV |
| chr15 | 79963372 | 79964372 | IV | chr11 | 53485802 | 53490032 | IV |
| chr15 | 79963373 | 79964373 | IV | chr1 | 135864839 | 135867465 | IV |
| chr2 | 116559678 | 116560883 | IV | chr9 | 62029969 | 62032442 | IV |
| chr17 | 8259638 | 8262074 | IV | chr9 | 118388965 | 118390841 | IV |
| chr2 | 116560589 | 116562952 | IV | chr9 | 118388318 | 118390635 | IV |
| chr2 | 116560885 | 116563192 | IV | chr12 | 99122553 | 99123557 | IV |
| chr2 | 116560940 | 116563328 | IV | chr11 | 24044157 | 24045159 | IV |
| chr16 | 28563660 | 28567140 | IV | chr7 | 81996184 | 81997186 | IV |
| chr4 | 143387859 | 143391276 | IV | chr14 | 120468791 | 120470314 | IV |
| chr4 | 110016205 | 110026059 | IV | chr12 | 28218916 | 28219917 | IV |
| chr6 | 117994926 | 117998693 | IV | chr15 | 31166959 | 31169085 | IV |
| chr11 | 33173296 | 33182467 | IV | chr15 | 31167011 | 31168441 | IV |
| chr10 | 82952823 | 82955639 | IV | chr15 | 31166394 | 31168138 | IV |
| chr18 | 76842232 | 76846368 | IV | chr5 | 135284084 | 135285084 | IV |
| chr18 | 76842579 | 76846207 | IV | chr16 | 35537567 | 35538567 | IV |
| chr2 | 116555404 | 116556432 | IV | chr10 | 80456901 | 80461522 | IV |
| chr2 | 116554634 | 116556600 | IV | chr14 | 46397949 | 46399782 | IV |
| chr2 | 116555345 | 116558876 | IV | chr13 | 31802092 | 31803907 | IV |
| chr2 | 116555242 | 116558344 | IV | chr1 | 39176063 | 39180235 | IV |
| chr2 | 116556437 | 116557734 | IV | chr5 | 33535777 | 33537326 | IV |
| chr2 | 116556631 | 116557631 | IV | chr2 | 170436435 | 170440163 | IV |
| chr2 | 131811738 | 131814450 | IV | chr13 | 56255677 | 56256677 | IV |
| chr1 | 133171413 | 133172950 | IV | chr13 | 56255497 | 56256506 | IV |
| chr4 | 114668035 | 114669036 | IV | chr7 | 36678174 | 36681081 | IV |
| chr11 | 76698813 | 76701289 | IV | chr7 | 36677717 | 36680569 | IV |
| chr2 | 31879895 | 31883568 | IV | chr7 | 36677503 | 36679265 | IV |
| chr16 | 91219174 | 91221087 | IV | chr5 | 147221284 | 147224620 | IV |
| chr17 | 56146547 | 56148623 | IV | chr5 | 147220391 | 147225043 | IV |
| chr1 | 34634526 | 34636616 | IV | chr5 | 147220668 | 147225593 | IV |
| chr1 | 34634967 | 34635967 | IV | chr5 | 147221579 | 147223414 | IV |
| chr9 | 100398494 | 100399665 | IV | chr5 | 147222041 | 147223489 | IV |
| chr7 | 35922144 | 35923450 | IV | chr7 | 49676209 | 49680352 | IV |
| chr11 | 96380062 | 96381062 | IV | chr7 | 49676372 | 49680279 | IV |
| chr10 | 73101076 | 73102100 | IV | chr19 | 59189531 | 59198165 | IV |
| chr19 | 45063771 | 45068438 | IV | chr19 | 59192318 | 59195616 | IV |
| chr19 | 45064167 | 45067599 | IV | chr19 | 59193173 | 59194173 | IV |
| chr4 | 149808327 | 149812589 | IV | chr5 | 75083105 | 75084227 | IV |
| chr2 | 93579200 | 93581080 | IV | chr5 | 75083059 | 75084059 | IV |
| chr5 | 119694167 | 119696782 | IV | chr12 | 84624388 | 84627111 | IV |
| chr1 | 119397813 | 119400001 | IV | chr4 | 134453357 | 134454372 | IV |
| chr7 | 49660710 | 49662870 | IV | chr2 | 84865019 | 84867598 | IV |
| chr10 | 77664234 | 77665236 | IV | chr12 | 118841563 | 118842563 | IV |
| chr4 | 150034225 | 150035825 | IV | chr17 | 85641007 | 85648313 | IV |
| chr4 | 115977495 | 115978495 | IV | chr8 | 48219402 | 48222056 | IV |
| chr4 | 115977486 | 115979160 | IV | chr2 | 172731687 | 172733359 | IV |
| chr3 | 117450739 | 117451739 | IV | chr17 | 85488568 | 85490711 | IV |
| chr15 | 102760096 | 102762818 | IV | chr6 | 23259276 | 23261762 | IV |
| chr13 | 69488269 | 69489269 | IV | chr2 | 38378441 | 38379441 | IV |
| chr6 | 64732105 | 64734326 | IV | chr6 | 23254731 | 23255734 | IV |
| chr11 | 96124866 | 96125866 | IV | chr17 | 85472770 | 85474134 | IV |
| chr14 | 32491497 | 32494421 | IV | chr17 | 85472283 | 85473399 | IV |
| chr14 | 32492575 | 32494767 | IV | chr17 | 85472983 | 85476716 | IV |
| chr9 | 89869815 | 89870945 | IV | chr17 | 85472358 | 85474718 | IV |
| chr9 | 89869485 | 89871331 | IV | chr5 | 101647881 | 101648928 | IV |
| chr18 | 42430154 | 42431474 | IV | chr19 | 45142489 | 45144440 | IV |
| chr11 | 98312809 | 98314850 | IV | chr4 | 153496148 | 153497675 | IV |
| chr2 | 73281154 | 73282154 | IV | chr10 | 77672929 | 77681945 | IV |
| chr11 | 66566606 | 66569586 | IV | chr17 | 56148896 | 56151606 | IV |
| chr5 | 108688715 | 108689715 | IV | chr17 | 56147872 | 56149799 | IV |
| chr9 | 89867222 | 89871206 | IV | chr4 | 122983420 | 122986112 | IV |
| chr9 | 89867716 | 89869370 | IV | chr14 | 67229055 | 67230308 | IV |
| chr5 | 98292578 | 98294199 | IV | chr10 | 19351311 | 19353171 | IV |
| chr10 | 19361118 | 19362744 | IV | chr4 | 115978693 | 115980687 | IV |
| chr4 | 21689333 | 21690342 | IV | chr4 | 115979134 | 115980535 | IV |
| chr4 | 115978406 | 115981567 | IV | chr4 | 114648114 | 114649728 | IV |
| chr11 | 119034632 | 119035945 | IV | chr4 | 114647988 | 114648996 | IV |
| chr11 | 119035177 | 119036177 | IV | chr4 | 114647936 | 114650374 | IV |
| chr11 | 119034379 | 119035770 | IV | chr4 | 114647976 | 114650903 | IV |
| chr11 | 119034586 | 119035586 | IV | chr4 | 114647674 | 114650746 | IV |
| chr11 | 119033800 | 119035521 | IV | chr4 | 114645755 | 114651069 | IV |
| chr17 | 36231465 | 36232523 | IV | chr4 | 114646099 | 114649813 | IV |
| chr17 | 36230274 | 36233468 | IV | chr19 | 44885901 | 44890181 | IV |
| chr17 | 36231272 | 36232562 | IV | chr17 | 83228411 | 83232498 | IV |
| chr3 | 149518866 | 149520825 | IV | chr5 | 120088456 | 120089931 | IV |
| chr1 | 154883547 | 154885538 | IV | chr5 | 120087652 | 120090293 | IV |
| chr9 | 54675990 | 54677882 | IV | chr4 | 89523887 | 89525602 | IV |
| chr1 | 6441580 | 6442580 | IV | chr4 | 89524166 | 89525607 | IV |
| chr6 | 85408057 | 85410352 | IV | chr4 | 89524528 | 89525992 | IV |
| chr6 | 85407494 | 85410061 | IV | chr4 | 89523041 | 89525005 | IV |
| chr4 | 115943227 | 115945200 | IV | chr17 | 35357252 | 35359926 | IV |
| chr2 | 91921707 | 91924677 | IV | chr17 | 35357235 | 35359925 | IV |
| chr2 | 91922238 | 91924135 | IV | chr17 | 35357627 | 35360126 | IV |
| chr2 | 91920525 | 91922286 | IV | chr17 | 35358021 | 35359925 | IV |
| chr10 | 13059693 | 13062683 | IV | chr17 | 35357553 | 35359575 | IV |
| chr10 | 13059160 | 13062454 | IV | chr17 | 35357134 | 35360816 | IV |
| chr10 | 13060821 | 13062470 | IV | chr17 | 35356086 | 35359925 | IV |
| chr6 | 88240985 | 88243037 | IV | chr17 | 35358536 | 35360837 | IV |
| chr6 | 88240430 | 88243750 | IV | chr10 | 7390399 | 7394503 | IV |
| chr18 | 75360587 | 75362020 | IV | chr10 | 7390347 | 7393757 | IV |
| chr4 | 109393395 | 109395721 | IV | chr10 | 7390822 | 7392570 | IV |
| chr2 | 118884227 | 118886826 | IV | chr10 | 7390114 | 7391759 | IV |
| chr10 | 80444903 | 80451684 | IV | chr10 | 7389837 | 7391418 | IV |
| chr10 | 80444917 | 80447800 | IV | chr10 | 7390755 | 7395438 | IV |
| chr10 | 80446151 | 80447650 | IV | chr10 | 7388873 | 7396681 | IV |
| chr5 | 101538158 | 101539523 | IV | chr4 | 46367862 | 46374015 | IV |
| chr5 | 101538150 | 101539514 | IV | chr13 | 56417414 | 56419906 | IV |
| chr4 | 114664801 | 114670653 | IV | chr15 | 35325796 | 35327538 | IV |
| chr4 | 114665145 | 114668994 | IV | chr17 | 36237146 | 36239080 | IV |
| chr4 | 114664251 | 114669541 | IV | chr11 | 95200142 | 95203333 | IV |
| chr4 | 114664903 | 114666973 | IV | chr11 | 95200593 | 95203708 | IV |
| chr4 | 114666151 | 114668461 | IV | chr11 | 95200122 | 95205103 | IV |
| chr4 | 114666471 | 114667471 | IV | chr9 | 98972559 | 98974225 | IV |
| chr5 | 67213185 | 67235435 | IV | chr3 | 154546630 | 154549341 | IV |
| chr5 | 67226306 | 67227306 | IV | chr2 | 172737839 | 172739611 | IV |
| chr6 | 64734276 | 64735644 | IV | chr4 | 46352110 | 46353636 | IV |
| chr6 | 64734631 | 64735636 | IV | chr13 | 48671682 | 48673610 | IV |
| chr6 | 64733310 | 64735674 | IV | chr2 | 172786513 | 172788231 | IV |
| chr2 | 172786759 | 172788874 | IV | chr17 | 3013135 | 3014135 | IV |
| chr13 | 48728962 | 48736332 | IV | chr13 | 53381624 | 53385084 | IV |
| chr13 | 48729387 | 48733915 | IV | chr9 | 88315272 | 88322083 | IV |
| chr13 | 48730828 | 48731828 | IV | chr1 | 167667315 | 167675101 | IV |
| chr11 | 64953236 | 64956879 | IV | chr4 | 114921403 | 114922416 | IV |
| chr7 | 3200731 | 3202509 | IV | chr16 | 61055406 | 61056457 | IV |
| chr16 | 17731594 | 17734074 | IV | chr16 | 61055420 | 61057377 | IV |
| chr13 | 31791963 | 31794862 | IV | chr18 | 3005311 | 3006661 | IV |
| chr15 | 35291978 | 35292996 | IV | chr18 | 3004684 | 3006836 | IV |
| chr5 | 119659467 | 119661885 | IV | chr2 | 181917723 | 181919038 | IV |
| chr5 | 119664647 | 119666851 | IV | chr2 | 181917471 | 181919230 | IV |
| chr5 | 37829108 | 37830808 | IV | chr2 | 181917447 | 181918472 | IV |
| chr3 | 154334774 | 154336726 | IV | chr3 | 8245528 | 8247499 | IV |
| chr1 | 78218320 | 78220146 | IV | chr3 | 8244441 | 8246973 | IV |
| chr1 | 91777326 | 91780675 | IV | chr15 | 75084798 | 75086425 | IV |
| chr14 | 61688764 | 61691803 | IV | chr15 | 75085207 | 75086751 | IV |
| chr14 | 61688821 | 61690568 | IV | chr15 | 75084631 | 75087099 | IV |
| chr3 | 3022017 | 3024041 | IV | chr18 | 3005868 | 3006874 | IV |
| chr3 | 3015073 | 3016890 | IV | chr2 | 181930303 | 181931900 | IV |
| chr3 | 3011605 | 3013738 | IV | chr2 | 181930329 | 181931356 | IV |
| chr17 | 21777707 | 21779720 | IV | chr2 | 182011784 | 182013944 | IV |
| chr5 | 8628249 | 8629249 | IV | chr17 | 36181488 | 36182531 | IV |
| chr4 | 89302574 | 89303574 | IV | chr17 | 36181394 | 36184455 | IV |
| chr12 | 116007629 | 116009368 | IV | chr17 | 36181512 | 36182771 | IV |
| chr11 | 3103976 | 3104977 | IV | chr17 | 36181362 | 36182847 | IV |
| chr10 | 57445228 | 57448880 | IV | chr17 | 36181519 | 36182815 | IV |
| chr10 | 57444837 | 57446622 | IV | chr17 | 36181480 | 36182865 | IV |
| chr1 | 171542303 | 171544548 | IV | chr17 | 36181490 | 36183513 | IV |
| chr4 | 89300151 | 89301331 | IV | chr17 | 36181497 | 36183582 | IV |
| chr4 | 89300151 | 89301151 | IV | chr7 | 47935613 | 47938581 | IV |
| chr17 | 36133786 | 36135874 | IV | chr6 | 18157699 | 18163236 | IV |
| chr17 | 36133669 | 36135297 | IV | chr9 | 17986836 | 17988795 | IV |
| chr2 | 11061353 | 11062353 | IV | chr10 | 57437938 | 57439674 | IV |
| chr6 | 126054275 | 126055275 | IV | chr6 | 21209646 | 21212250 | IV |
| chr6 | 67513178 | 67515033 | IV | chrX | 101694925 | 101696799 | IV |
| chr5 | 98190823 | 98192261 | IV | chr16 | 18681091 | 18682913 | IV |
| chr4 | 147598078 | 147599081 | IV | chr19 | 11877606 | 11878913 | IV |
| chr11 | 96205109 | 96210021 | IV | chr12 | 110317029 | 110320491 | IV |
| chr17 | 85660972 | 85673674 | IV | chr2 | 116550902 | 116553412 | IV |
| chr2 | 18690999 | 18692993 | IV | chr12 | 110319147 | 110321325 | IV |
| chr17 | 36213941 | 36215594 | IV | chr17 | 36203290 | 36212761 | IV |
| chr10 | 114824360 | 114826908 | IV | chr9 | 89874649 | 89886318 | IV |
| chr10 | 73379012 | 73380221 | IV | chr17 | 22875494 | 22877825 | IV |
| chr17 | 36135982 | 36139687 | IV | chr2 | 116547329 | 116548349 | IV |
| chr2 | 116548794 | 116558522 | IV | chr2 | 11872988 | 11873993 | IV |
| chr2 | 116548361 | 116554300 | IV | chr11 | 49588947 | 49591129 | IV |
| chr2 | 116544177 | 116564653 | IV | chr15 | 86194184 | 86196243 | IV |
| chr17 | 36076834 | 36079730 | IV | chr4 | 3162691 | 3166509 | IV |
| chr16 | 16197505 | 16198506 | IV | chr18 | 3112479 | 3116898 | IV |
| chr17 | 36093147 | 36095636 | IV | chr12 | 18135430 | 18137555 | IV |
| chr17 | 36093059 | 36095581 | IV | chr10 | 7488326 | 7493776 | IV |
| chr17 | 36093113 | 36094261 | IV | chr9 | 24951782 | 24953650 | IV |
| chr17 | 13551234 | 13554126 | IV | chr1 | 171083539 | 171087723 | IV |
| chr15 | 75084626 | 75085628 | IV | chr5 | 26067920 | 26072400 | IV |
| chr3 | 5859539 | 5860540 | IV | chr5 | 26067513 | 26072396 | IV |
| chr3 | 3032044 | 3033280 | IV | chr5 | 26068809 | 26071631 | IV |
| chr3 | 3032124 | 3033274 | IV | chr12 | 115555477 | 115558972 | IV |
| chr17 | 36064976 | 36068689 | IV | chr12 | 115555441 | 115557747 | IV |
| chr17 | 36064992 | 36067511 | IV | chr12 | 115635482 | 115642910 | IV |
| chr17 | 36065055 | 36067205 | IV | chr12 | 115635165 | 115643703 | IV |
| chr17 | 35354991 | 35357112 | IV | chrX | 137102625 | 137108125 | IV |
| chr17 | 35355098 | 35356423 | IV | chrX | 137097194 | 137101671 | IV |
| chr12 | 116008756 | 116013215 | IV | chrX | 137125016 | 137129931 | IV |
| chr4 | 147516278 | 147520430 | IV | chr5 | 14960243 | 14962687 | IV |
| chr11 | 3198329 | 3199329 | IV | chr17 | 36207325 | 36208608 | IV |
| chr11 | 3197295 | 3199081 | IV | chr12 | 115618204 | 115619432 | IV |
| chr3 | 3007595 | 3013693 | IV | chr14 | 124800835 | 124802567 | IV |
| chr13 | 119611894 | 119615490 | IV | chr12 | 115797460 | 115798460 | IV |
| chr7 | 47931811 | 47935119 | IV | chr5 | 15496969 | 15499005 | IV |
| chr12 | 116005734 | 116008989 | IV | chr5 | 15496533 | 15498662 | IV |
| chr4 | 145560826 | 145564324 | IV | chr12 | 115808317 | 115811035 | IV |
| chr4 | 3057608 | 3070576 | IV | chr12 | 115807564 | 115809249 | IV |
| chr4 | 3078557 | 3086780 | IV | chr12 | 115551722 | 115558904 | IV |
| chr8 | 20948036 | 20951110 | IV | chr5 | 15003811 | 15010112 | IV |
| chr17 | 15574526 | 15576915 | IV | chr8 | 20986326 | 20994317 | IV |
| chr12 | 115061407 | 115063367 | IV | chr7 | 47727159 | 47743852 | IV |
| chr4 | 147522902 | 147525112 | IV | chr6 | 67672075 | 67684570 | IV |
| chr4 | 147522929 | 147525392 | IV | chr12 | 115521502 | 115524746 | IV |
| chr4 | 147523258 | 147526238 | IV | chr12 | 115516609 | 115529572 | IV |
| chr4 | 147524239 | 147525239 | IV | chr5 | 15050735 | 15055766 | IV |
| chr12 | 115636114 | 115639113 | IV | chr5 | 26009354 | 26012163 | IV |
| chr12 | 115635082 | 115638855 | IV | chr5 | 26074297 | 26078079 | IV |
| chr12 | 115634535 | 115638745 | IV | | | | |
| chr12 | 115635115 | 115637695 | IV | | | | |
| chr4 | 145563699 | 145565604 | IV | | | | |
| chr4 | 145563638 | 145565494 | IV | | | | |

Taken together, the PRC2 dependent chromatin connectivity configures the genome structures as the transcription silencing foci in the pluripotent genomes. Within the interaction networks, the PRC2 bound DREs can act as transcription silencers in a target-specific way to maintain the lineage specification genes in a repressive but poised chromatin state. Upon differentiation, these DREs can either remain bound by PRC2 in the silencing foci or convert into tissue specific enhancers, and the transitions between two regulatory states would be dependent on the chromatin conformation and the expression of stage- or tissue-specific transcription factors (FIG. 8E).

### Discussion—Examples 2-4

In the experiments described herein, PRC2 chromatin interaction analysis was used to reveal the widespread, highly precise and remarkably complex silencer associated chromatin connectivity networks in mESC genome. Acting as a repressor complex, PRC2 bound to the silencer regulatory elements to induce chromatin compaction and sequester developmentally regulated genes into the condensed, sub-nuclear microenvironments where transcription silencing can be facilitated by increasing the local concentration of specific repressive factors, PRC2 complexes and coregulated gene clusters. Within the silencing hubs, these silencers are presumed to function as the nucleation sites to initiate extensive chromatin looping.

In the last decade, transcriptional enhancers have emerged as the dominant class of regulatory elements in the non-coding portion of the mammalian genome [Consortium, E. P. Nature 489, 57-74 (2012)]. The experiments and results presented herein, provide a dual-activity model that provide genomes with maximal versatility in expression regulation. The non-coding regulatory sequences can function as both enhancers and silencers and the dynamic transcriptional regulatory activities of these functional elements are critically dependent on the nature of associated protein complexes, local sequence context and chromatin conformation. In contrast to thousands of protein-encoding gene knockouts in mice [Guan, C., et al., Genesis 48, 73-85 (2010) and Lloyd, K. C. Ann N Y Acad Sci 1245, 24-6 (2011)], only few of the regulatory elements have been subjected to knockout analysis [Osterwalder, M. et al. Nature 554, 239-243 (2018); Shim, S., et al., Nature 486, 74-9 (2012); and Sur, I. K. et al. Science 338, 1360-3 (2012)]. It is noteworthy that the pleiotropic patterns of phenotypical aberration associated with the deletion in PRC2-bound silencers highlight the influence of these PRC2-bound silencers in multiple lineages of organismal development. This is similar to the effects of numerous variants found in the noncoding regulatory elements measured by the GWAS studies in many human diseases. Moreover, such results may have implications in the mechanisms involved in the silencing of tumor suppressor genes which predispose cells to tumor progression [Kazanets, A., et al., Biochim Biophys Acta 1865, 275-88 (2016)]. The delineation of silencer sequence contexts, their distribution, and diversity underscores the versatility of epigenetic-based transcription regulation. Studies have now provided a basis for targeted re-expression of epigenetically silenced genes in therapeutics.

### Example 5

Experiments are performed in which a candidate agent for inhibiting cancer is identified. In the experiments, a means that includes a ChIA-PET method is used to identify a gene regulation system (GRS). An activity of the GRS includes a physical interaction between a gene regulator element, a gene regulator complex, and a gene modifier element in a cancer cell. In addition, a gene whose transcription is modified by the detected gene interaction is identified as a cancer-inducer gene or a cancer-suppressor gene. An effect of the activity of the GRS on repression and de-repression of the transcription of the identified gene is determined. It is determined that:
(a) if the identified gene is a cancer-suppressor gene and the effect of the activity of the GRS is de-repression of the transcription of the identified gene, the activity of the GRS inhibits the cancer;
(b) if the identified gene is a cancer-suppressor gene and the effect of the activity of the GRS is repression of the transcription of the identified gene, reducing the activity of the GRS inhibits the cancer;
(c) if the identified gene is a cancer-inducer gene and the effect of the activity of the GRS is repression of the transcription of the identified gene, the activity of the GRS inhibits the cancer;
(d) if the identified gene is a cancer-inducer gene and the effect of the activity of the GRS is de-repression of the transcription of the identified gene, reducing the activity of the GRS inhibits the cancer.
   Next, one or more candidate agents that modify an activity of the GRS are identified and based on (a)-(d) above, an agent is selected that has a desired effect on the activity of the GRS.

In a circumstance described in (a), the identified gene is a cancer-suppressor gene and a selected agent inhibits GRS activity, which reduces the repression of the transcription of the identified gene and inhibits the cancer. In some embodiments, in a circumstance described in (b), the identified gene is a cancer-suppressor gene and a selected agent inhibits GRS activity, which increases the transcription of the identified gene and inhibits the cancer. In some embodiments, in a circumstance described in (c), the identified gene is a cancer-inducer gene and a selected agent increases GRS activity, which increases the repression of the transcription of the identified gene and inhibits the cancer. In some embodiments, in a circumstance described in (d), the identified gene is a cancer-inducer gene and a selected agent inhibits GRS activity, which reduces the de-repression of the transcription of the identified gene and inhibits the cancer.

At least one selected agent is administered to a subject having the cancer as a treatment for the cancer and the cancer is effectively treated.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method of identifying a candidate agent for modulating a condition; the method comprising:
(a) detecting, with a means comprising a ChIA-PET method, a gene regulation system (GRS), wherein an activity of the GRS comprises a physical interaction between a transcription-factor binding (TFB) element, a transcription factor (TF) complex, and a gene modifier element in a cell;
(b) identifying a target gene whose transcription is modified by the activity of the GRS as a condition-inducer gene or a condition-suppressor gene;
(c) determining an effect of the activity of the GRS on repression and de-repression of the transcription of the identified target gene, wherein:
(i) if the target gene is a condition-suppressor gene and the effect of the activity of the GRS is de-repression of the transcription of the target gene, the activity of the GRS inhibits the condition;
(ii) if the target gene is a condition-suppressor gene and the effect of the activity of the GRS is repression of the transcription of the target gene, reducing the activity of the GRS inhibits the condition;
(iii) if the target gene is a condition-inducer gene and the effect of the activity of the GRS is repression of the transcription of the target gene, the activity of the GRS inhibits the condition;
(iv) if the target gene is a condition-inducer gene and the effect of the activity of the GRS is de-repression of the transcription of the target gene, reducing the activity of the GRS inhibits the condition; and
(d) identifying one or more candidate agents that modify the activity of the GRS.

2. The method of claim 1, wherein the TFC comprises 1, 2, 3, or more subunits, wherein the physical interaction between the TFB element, the TF complex, and the gene modifier element comprises an interaction between at least one of the subunits and the TFB element and an interaction between at least one of the subunits and the gene modifier element.

3. The method of claim 1, wherein the TFB element is a gene silencer element.

4. The method of claim 1, wherein the TF complex comprises a polycomb repressor complex 2 (PRC2).

5. The method of claim 4, wherein the GRS activity transcriptionally represses expression of the target gene.

6. The method of claim 1, wherein the TFB element is a gene activator element.

7. The method of claim 6, wherein the GRS activity transcriptionally de-represses expression of the target gene.

8. The method of claim 1, wherein the condition comprises at least one of: a cancer, cell differentiation, cell de-differentiation, embryonic development, development, organ development, cell death, cell division, and a genetic disease.

9. The method of claim 1, wherein the condition is a cancer and the target gene is a cancer-inducer gene.

10. The method of claim 1, wherein the condition is a cancer and the target gene is a cancer suppressor gene.

11. The method of claim 1, further comprising identifying a function of the identified target gene.

12-13. (canceled)

14. The method of claim 1, wherein the cell is a cancer cell.

15-17. (canceled)

18. The method of claim 1, further comprising determining a level of transcription of the target gene and optionally comparing the determined level to a control level of transcription.

19. The method of claim 18, further comprising determining an effect of one of the candidate agent on the determined level of transcription of the target gene, and optionally comparing the determined level to a control level of transcription, and optionally wherein determining the effect of one of the candidate agents comprises contacting the cell with a composition comprising the candidate agent.

20. (canceled)

21. The method of claim 1, further comprising, testing the one or more identified candidate agents of claim 1(d) as a determination of the presence or absence of an inhibitory effect of the agent(s) on the condition.

22. The method of claim 1, further comprising testing the one or more identified candidate agent of claim 1(d) as a determination of the presence or absence of an enhancing effect of the agent(s) on the condition.

23. A method of identifying a candidate agent for inhibiting a cancer; the method comprising:
(a) detecting, with a means comprising a ChIA-PET method, a gene regulation system (GRS), wherein an activity of the GRS comprises a physical interaction between a transcription-factor binding (TFB) element, a transcription factor (it) complex, and a gene modifier element in a cancer cell;
(b) identifying a target gene whose transcription is modified by the activity of the GRS as a cancer-inducer gene or a cancer-suppressor gene;
(c) determining an effect of the activity of the GRS on repression and de-repression of the transcription of the identified target gene, wherein:
(i) if the target gene is a cancer-suppressor gene and the effect of the activity of the GRS is de-repression of the transcription of the target gene, the activity of the GRS inhibits the cancer;
(ii) if the target gene is a cancer-suppressor gene and the effect of the activity of the GRS is repression of the transcription of the target gene, reducing the activity of the GRS inhibits the cancer;
(iii) if the target gene is a cancer-inducer gene and the effect of the activity of the GRS is repression of the transcription of the target gene, the activity of the GRS inhibits the cancer;
(iv) if the target gene is a cancer-inducer gene and the effect of the activity of the GRS is de-repression of the transcription of the target gene, reducing the activity of the GRS inhibits the cancer; and
(d) identifying one or more candidate agents that modify the activity of the GRS.

24. The method of claim 23, wherein the TFC comprises 1, 2, 3, or more subunits, wherein the physical interaction between the TFB element, the TF complex, and the gene modifier element comprises an interaction between at least one of the subunits and the TFB element and an interaction between at least one of the subunits and the gene modifier element.

25. The method of claim 23, wherein the TFB element is a gene silencer element.

26. The method of claim 23, wherein the TF complex comprises a polycomb repressor complex 2 (PRC2).

27-40. (canceled)


SEQUENCE LISTING (134 sequences)

SEQ ID NO: 1 - DNA, 13 bp, Mus musculus
agatcggaagagc

SEQ ID NO: 2 - DNA, 20 bp, Mus musculus
tttcgccgcggaagcagatt

SEQ ID NO: 3 - DNA, 20 bp, Mus musculus
ggctctcacaggtatagacc

SEQ ID NO: 4 - DNA, 20 bp, Mus musculus
gtgctgtcacgctaaagaat

SEQ ID NO: 5 - DNA, 20 bp, Mus musculus
gtcatttgaacatggctacc

SEQ ID NO: 6 - DNA, 20 bp, Mus musculus
ggaccctacacagataggct

SEQ ID NO: 7 - DNA, 20 bp, Mus musculus
ttcctgaagtctgttgtcgc

SEQ ID NO: 8 - DNA, 20 bp, Mus musculus
gactaagccctctttcccca

SEQ ID NO: 9 - DNA, 20 bp, Mus musculus
gctgctggcccacaatctgg

SEQ ID NO: 10 - DNA, 20 bp, Mus musculus
agccaccagcttatgcccgg

SEQ ID NO: 11 - DNA, 20 bp, Mus musculus
tgtggtccccgccataatcc

SEQ ID NO: 12 - DNA, 20 bp, Mus musculus
gaccccaggcacagtcttat

SEQ ID NO: 13 - DNA, 20 bp, Mus musculus
gtcaaccgtcttggcttcga

SEQ ID NO: 14 - DNA, 20 bp, Mus musculus
ttaagtgtcaccggtagcca

SEQ ID NO: 15 - DNA, 20 bp, Mus musculus
actcttgctgaccatcaccg

SEQ ID NO: 16 - DNA, 20 bp, Mus musculus
ctgctccatatccttggagc

SEQ ID NO: 17 - DNA, 20 bp, Mus musculus
caatctctgtagtgaaggta

SEQ ID NO: 18 - DNA, 20 bp, Mus musculus
atttgctatcaggctaaggc

SEQ ID NO: 19 - DNA, 20 bp, Mus musculus
catcgtattcttttgcatcc

SEQ ID NO: 20 - DNA, 20 bp, Mus musculus
gctcaaggcgcgaacacccg

SEQ ID NO: 21 - DNA, 20 bp, Mus musculus
aggacagcctgcgtacatca

SEQ ID NO: 22 - DNA, 20 bp, Mus musculus
gctcggcgctagtactattc

SEQ ID NO: 23 - DNA, 20 bp, Mus musculus
gtccatgggattctactcct

SEQ ID NO: 24 - DNA, 20 bp, Mus musculus
taccctaggccctggtgaac

SEQ ID NO: 25 - DNA, 20 bp, Mus musculus
ccgtgcgtgtcacaccgtac

SEQ ID NO: 26 - DNA, 20 bp, Mus musculus
aagtaactaggttgtctgat

SEQ ID NO: 27 - DNA, 20 bp, Mus musculus
catccatgggatgctattta

SEQ ID NO: 28 - DNA, 20 bp, Mus musculus
actctgcgttgaaccattct

SEQ ID NO: 29 - DNA, 20 bp, Mus musculus
gcaagcttatcatacaactg

SEQ ID NO: 30 - DNA, 20 bp, Mus musculus
gctccggtgttgacttacct

SEQ ID NO: 31 - DNA, 20 bp, Mus musculus
gcagcgtgcggggaatgagt

SEQ ID NO: 32 - DNA, 20 bp, Mus musculus
ccaataagtcactaggggcg

SEQ ID NO: 33 - DNA, 20 bp, Mus musculus
gtgatgcataaatgcagtgc

SEQ ID NO: 34 - DNA, 20 bp, Mus musculus
ggctagacgagaactagtga

SEQ ID NO: 35 - DNA, 20 bp, Mus musculus
ttggagaagggtagtaagac

SEQ ID NO: 36 - DNA, 20 bp, Mus musculus
taggcctttcagatgatgcc

SEQ ID NO: 37 - DNA, 20 bp, Mus musculus
ctcttcgccgcacagtactc

SEQ ID NO: 38 - DNA, 20 bp, Mus musculus
tcagagaactctttttggac

SEQ ID NO: 39 - DNA, 20 bp, Mus musculus
ccgtcgcttttcattccggg

SEQ ID NO: 40 - DNA, 20 bp, Mus musculus
ctagacccttaagacatacg

SEQ ID NO: 41 - DNA, 20 bp, Mus musculus
agcgatcaaggaactctggg

SEQ ID NO: 42 - DNA, 21 bp, Artificial Sequence
  Synthetic Oligonucleotide
acgatgctaattacggggtgc

SEQ ID NO: 43 - DNA, 21 bp, Artificial Sequence
  Synthetic Oligonucleotide
gtgtctgtagctcccatccct

SEQ ID NO: 44 - DNA, 25 bp, Artificial Sequence
  Synthetic Oligonucleotide
ctccttaaacatgctatggcactgc

SEQ ID NO: 45 - DNA, 23 bp, Artificial Sequence
  Synthetic Oligonucleotide
gagagagtgatttctggcatggg

SEQ ID NO: 46 - DNA, 26 bp, Artificial Sequence
  Synthetic Oligonucleotide
ctggtacccaccatatacataggaca

SEQ ID NO: 47 - DNA, 23 bp, Artificial Sequence
  Synthetic Oligonucleotide
atgtaaccctggcagagatctgc

SEQ ID NO: 48 - DNA, 22 bp, Artificial Sequence
  Synthetic Oligonucleotide
ggcatgaaggtgtacatgcaga

SEQ ID NO: 49 - DNA, 21 bp, Artificial Sequence
  Synthetic Oligonucleotide
aaggcacagctgtattgcctg

SEQ ID NO: 50 - DNA, 23 bp, Artificial Sequence
  Synthetic Oligonucleotide
tgacgttcccatttcctgacctt

SEQ ID NO: 51 - DNA, 23 bp, Artificial Sequence
  Synthetic Oligonucleotide
ccatatgatctggctccttccac

SEQ ID NO: 52 - DNA, 24 bp, Artificial Sequence
  Synthetic Oligonucleotide
agattctaggcaggagctctacca

SEQ ID NO: 53 - DNA, 22 bp, Artificial Sequence
  Synthetic Oligonucleotide
ggttgttgttgctgctgctact

SEQ ID NO: 54 - DNA, 24 bp, Artificial Sequence
  Synthetic Oligonucleotide
tctcagatgctaagaggatccctc

SEQ ID NO: 55 - DNA, 23 bp, Artificial Sequence
  Synthetic Oligonucleotide
cttagggttcatcctgggtctct

SEQ ID NO: 56 - DNA, 24 bp, Artificial Sequence
  Synthetic Oligonucleotide
ctcactgatagtggccatcacaag

SEQ ID NO: 57 - DNA, 24 bp, Artificial Sequence
  Synthetic Oligonucleotide
gatggcacacacctttgattccag

SEQ ID NO: 58 - DNA, 23 bp, Artificial Sequence
  Synthetic Oligonucleotide
gactcaggcagtttacctggatg

SEQ ID NO: 59 - DNA, 24 bp, Artificial Sequence
  Synthetic Oligonucleotide
gcagtcagtgctcttaaccactga

SEQ ID NO: 60 - DNA, 23 bp, Artificial Sequence
  Synthetic Oligonucleotide
ggaactgggaacctaaactgtgg

SEQ ID NO: 61 - DNA, 22 bp, Artificial Sequence
  Synthetic Oligonucleotide
agcttaaagcggctactgcatg

SEQ ID NO: 62 - DNA, 24 bp, Artificial Sequence
  Synthetic Oligonucleotide
gagaggggaagggagttatcgttt

SEQ ID NO: 63 - DNA, 22 bp, Artificial Sequence
  Synthetic Oligonucleotide
ttccactaggagtgtccatggt

SEQ ID NO: 64 - DNA, 24 bp, Artificial Sequence
  Synthetic Oligonucleotide
tggtagaaagaagacagcctcagc

SEQ ID NO: 65 - DNA, 26 bp, Artificial Sequence
  Synthetic Oligonucleotide
ggctgtgagctgtagagttatctagg

SEQ ID NO: 66 - DNA, 24 bp, Artificial Sequence
  Synthetic Oligonucleotide
acatcctctgtaggacttagggga

SEQ ID NO: 67 - DNA, 21 bp, Artificial Sequence
  Synthetic Oligonucleotide
ttcacactgtcattgtccgcg

SEQ ID NO: 68 - DNA, 23 bp, Artificial Sequence
  Synthetic Oligonucleotide
tctgtgcctcactgttgatgaga

SEQ ID NO: 69 - DNA, 24 bp, Artificial Sequence
  Synthetic Oligonucleotide
ggaagcaggttagtcctgtagtcc

SEQ ID NO: 70 - DNA, 23 bp, Artificial Sequence
  Synthetic Oligonucleotide
agttagcagctctcatgaaccca

SEQ ID NO: 71 - DNA, 23 bp, Artificial Sequence
  Synthetic Oligonucleotide
acataccacagagggcattggat

SEQ ID NO: 72 - DNA, 24 bp, Artificial Sequence
  Synthetic Oligonucleotide
tgtggaaagagtgttgcatcatgg

SEQ ID NO: 73 - DNA, 21 bp, Artificial Sequence
  Synthetic Oligonucleotide
tggcagagtgatactgctgca

SEQ ID NO: 74 - DNA, 24 bp, Artificial Sequence
  Synthetic Oligonucleotide
ctgggccatatctattggtagcct

SEQ ID NO: 75 - DNA, 25 bp, Artificial Sequence
  Synthetic Oligonucleotide
ggtgtagaaagtgacagactccaca

SEQ ID NO: 76 - DNA, 24 bp, Artificial Sequence
  Synthetic Oligonucleotide
acacagagtatgatccgatccacc

SEQ ID NO: 77 - DNA, 23 bp, Artificial Sequence
  Synthetic Oligonucleotide
tagatgccatttgtagctcagcc

SEQ ID NO: 78 - DNA, 23 bp, Artificial Sequence
  Synthetic Oligonucleotide
caactttggagaccagagactgc

SEQ ID NO: 79 - DNA, 24 bp, Artificial Sequence
  Synthetic Oligonucleotide
ttcatccctccttttcctgtaccc

SEQ ID NO: 80 - DNA, 25 bp, Artificial Sequence
  Synthetic Oligonucleotide
cagatacagaagctcaatcccgaag

SEQ ID NO: 81 - DNA, 23 bp, Artificial Sequence
  Synthetic Oligonucleotide
catcctgaagggcatgagggata

SEQ ID NO: 82 - DNA, 22 bp, Artificial Sequence
  Synthetic Oligonucleotide
ttcctgtaggctgtagggtgaa

SEQ ID NO: 83 - DNA, 22 bp, Artificial Sequence
  Synthetic Oligonucleotide
agactccaggacattccctgat

SEQ ID NO: 84 - DNA, 22 bp, Artificial Sequence
  Synthetic Oligonucleotide
cctcagaccactggagccattt

SEQ ID NO: 85 - DNA, 23 bp, Artificial Sequence
  Synthetic Oligonucleotide
ttctatcaaagagtcgccagggg

SEQ ID NO: 86 - DNA, 20 bp, Artificial Sequence
  Synthetic Oligonucleotide
aagccctctgtgatgaccct

SEQ ID NO: 87 - DNA, 23 bp, Artificial Sequence
  Synthetic Oligonucleotide
tctctacttaggggcagtgagac

SEQ ID NO: 88 - DNA, 24 bp, Artificial Sequence
  Synthetic Oligonucleotide
cctgttcatgatgcatctctgtca

SEQ ID NO: 89 - DNA, 22 bp, Artificial Sequence
  Synthetic Oligonucleotide
agagaagagctgctgtgcatgt

SEQ ID NO: 90 - DNA, 22 bp, Artificial Sequence
  Synthetic Oligonucleotide
gcttcctgatcagatggcaaca

SEQ ID NO: 91 - DNA, 23 bp, Artificial Sequence
  Synthetic Oligonucleotide
actctaagccatagttgcccacc

SEQ ID NO: 92 - DNA, 23 bp, Artificial Sequence
  Synthetic Oligonucleotide
tgctgactagagtctctcaggga

SEQ ID NO: 93 - DNA, 23 bp, Artificial Sequence
  Synthetic Oligonucleotide
ccactgatgaaggctcttggatt

SEQ ID NO: 94 - DNA, 23 bp, Artificial Sequence
  Synthetic Oligonucleotide
atggacatatgcgaagggtgagt

SEQ ID NO: 95 - DNA, 26 bp, Artificial Sequence
  Synthetic Oligonucleotide
ggacacattcaatcatccaaatccct

SEQ ID NO: 96 - DNA, 26 bp, Artificial Sequence
  Synthetic Oligonucleotide
cccactcttcctgttacactatctgc

SEQ ID NO: 97 - DNA, 24 bp, Artificial Sequence
  Synthetic Oligonucleotide
ccgttacatctgaacctgtgttga

SEQ ID NO: 98 - DNA, 23 bp, Artificial Sequence
  Synthetic Oligonucleotide
ggtgctagggacagaaatggaac

SEQ ID NO: 99 - DNA, 24 bp, Artificial Sequence
  Synthetic Oligonucleotide
agcagaggtagagcttccttgttg

SEQ ID NO: 100 - DNA, 24 bp, Artificial Sequence
  Synthetic Oligonucleotide
ggaatagggaaagccatgtgttgg

SEQ ID NO: 101 - DNA, 21 bp, Artificial Sequence
  Synthetic Oligonucleotide
aggaaccgaaagtggtctgca

SEQ ID NO: 102 - DNA, 20 bp, Artificial Sequence
  Synthetic Oligonucleotide
aaaggggcctcatactggca

SEQ ID NO: 103 - DNA, 24 bp, Artificial Sequence
  Synthetic Oligonucleotide
ccctctttccatccaggaaactca

SEQ ID NO: 104 - DNA, 22 bp, Artificial Sequence
  Synthetic Oligonucleotide
gccagcaggaaccaaatctgtt

SEQ ID NO: 105 - DNA, 23 bp, Artificial Sequence
  Synthetic Oligonucleotide
cgaagtacgagcaaggtaagtcg

SEQ ID NO: 106 - DNA, 23 bp, Artificial Sequence
  Synthetic Oligonucleotide
ttaccatactagtcacccggcag

SEQ ID NO: 107 - DNA, 20 bp, Artificial Sequence
  Synthetic Oligonucleotide
gcctgcacagttctgcactt

SEQ ID NO: 108 - DNA, 24 bp, Artificial Sequence
  Synthetic Oligonucleotide
ctgtcatcttccacagacctgtca

SEQ ID NO: 109 - DNA, 23 bp, Artificial Sequence
  Synthetic Oligonucleotide
atggcaagcaagcctcaaactag

SEQ ID NO: 110 - DNA, 21 bp, Artificial Sequence
  Synthetic Oligonucleotide
atcttcccaagcggtccacat

SEQ ID NO: 111 - DNA, 22 bp, Artificial Sequence
  Synthetic Oligonucleotide
aagctggacttctcccaagctt

SEQ ID NO: 112 - DNA, 23 bp, Artificial Sequence
  Synthetic Oligonucleotide
gcaaaactctacctctgagccac

SEQ ID NO: 113 - DNA, 24 bp, Artificial Sequence
  Synthetic Oligonucleotide
cacaaaataacagcaagctcccct

SEQ ID NO: 114 - DNA, 22 bp, Artificial Sequence
  Synthetic Oligonucleotide
tggctctcctcagaagcatgat

SEQ ID NO: 115 - DNA, 23 bp, Artificial Sequence
  Synthetic Oligonucleotide
ggattgaacccaaggctttgagc

SEQ ID NO: 116 - DNA, 24 bp, Artificial Sequence
  Synthetic Oligonucleotide
agctctaaggccagtggatatacc

SEQ ID NO: 117 - DNA, 25 bp, Artificial Sequence
  Synthetic Oligonucleotide
gacgactctgaattctgctcattgg

SEQ ID NO: 118 - DNA, 22 bp, Artificial Sequence
  Synthetic Oligonucleotide
tggtgctttgaggaagcatcct

SEQ ID NO: 119 - DNA, 24 bp, Artificial Sequence
  Synthetic Oligonucleotide
gcttcccaatcaggaatatcgacc

SEQ ID NO: 120 - DNA, 22 bp, Artificial Sequence
  Synthetic Oligonucleotide
tgagcacaccattcagttgtgc

SEQ ID NO: 121 - DNA, 23 bp, Artificial Sequence
  Synthetic Oligonucleotide
gaactccaggtttcaccgaagag

SEQ ID NO: 122 - DNA, 24 bp, Artificial Sequence
  Synthetic Oligonucleotide
agggtggcagactgtcacagtctc

SEQ ID NO: 123 - DNA, 40 bp, Artificial Sequence
  Synthetic Oligonucleotide
ggaggggctcaaggtggctctcacaggtatagaccggggc

SEQ ID NO: 124 - DNA, 40 bp, Artificial Sequence
  Synthetic Oligonucleotide
ccccagtccatgggattctactccttttggaggcagcaat

SEQ ID NO: 125 - DNA, 40 bp, Artificial Sequence
  Synthetic Oligonucleotide
cctccggtcccaccagtcatttgaacatggctaccatcca

SEQ ID NO: 126 - DNA, 40 bp, Artificial Sequence
  Synthetic Oligonucleotide
tctacccgtgcgtgtcacaccgtacagggcgagatgtcat

SEQ ID NO: 127 - DNA, 40 bp, Artificial Sequence
  Synthetic Oligonucleotide
gaaatggagggtccaggaccctacacagataggcttaatc

SEQ ID NO: 128 - DNA, 40 bp, Artificial Sequence
  Synthetic Oligonucleotide
aggggaagtaactaggttgtctgatgggttcgttggcttc

SEQ ID NO: 129 - DNA, 40 bp, Artificial Sequence
  Synthetic Oligonucleotide
ccacaccccgccaaagctgctggcccacaatctgggggag

SEQ ID NO: 130 - DNA, 40 bp, Artificial Sequence
  Synthetic Oligonucleotide
gacctgcaagcttatcatacaactgtggcctcccttccac

SEQ ID NO: 131 - DNA, 40 bp, Artificial Sequence
  Synthetic Oligonucleotide
ggaggtggctatctggtcaaccgtcttggcttcgagggaa

SEQ ID NO: 132 - DNA, 40 bp, Artificial Sequence
  Synthetic Oligonucleotide
agccagtgatgcataaatgcagtgcgcatgtgcaggaagc

SEQ ID NO: 133 - DNA, 39 bp, Artificial Sequence
  Synthetic Oligonucleotide
cgggggagcaccctgctcaaggcgcgaacacccgggacg

SEQ ID NO: 134 - DNA, 40 bp, Artificial Sequence
  Synthetic Oligonucleotide
cactactagacccttaagacatacgaggttgaccgtaaaa